SEQUENCE LISTING (93 sequences)

SEQ ID NO: 1 - DNA, 21 bp, synthetic construct
  source 1..21: mol_type = other DNA
aaatccttccagagtaacaat

SEQ ID NO: 2 - DNA, 18 bp, synthetic construct
  source 1..18: mol_type = other DNA
aaccgttgtcctgagccg

SEQ ID NO: 3 - DNA, 20 bp, synthetic construct
  source 1..20: mol_type = other DNA
aaccgttgtcctgagccgtc

SEQ ID NO: 4 - DNA, 22 bp, synthetic construct
  source 1..22: mol_type = other DNA
aacgagaccttaacctgctaaa

SEQ ID NO: 5 - DNA, 19 bp, synthetic construct
  source 1..19: mol_type = other DNA
aagctcagcaacatgaacc

SEQ ID NO: 6 - DNA, 19 bp, synthetic construct
  source 1..19: mol_type = other DNA
aagctcagcaacatgaagg

SEQ ID NO: 7 - DNA, 30 bp, synthetic construct
  source 1..30: mol_type = other DNA
acaaaatggccaattcattcaatgaattga

SEQ ID NO: 8 - DNA, 21 bp, synthetic construct
  source 1..21: mol_type = other DNA
acctgctaaatagacataaga

SEQ ID NO: 9 - DNA, 23 bp, synthetic construct
  source 1..23: mol_type = other DNA
actgcaacaactctaatatacac

SEQ ID NO: 10 - DNA, 29 bp, synthetic construct
  source 1..29: mol_type = other DNA
agacgaactactgcgaaagcatttgccaa

SEQ ID NO: 11 - DNA, 22 bp, synthetic construct
  source 1..22: mol_type = other DNA
agcctgctaaatagacataaga

SEQ ID NO: 12 - DNA, 21 bp, synthetic construct
  source 1..21: mol_type = other DNA
aggaccctccgatcggttgca

SEQ ID NO: 13 - DNA, 20 bp, synthetic construct
  source 1..20: mol_type = other DNA
atctaatcgtcttcaaaccc

SEQ ID NO: 14 - DNA, 21 bp, synthetic construct
  source 1..21: mol_type = other DNA
attgttactctggaaggattt

SEQ ID NO: 15 - skipped

SEQ ID NO: 16 - DNA, 25 bp, synthetic construct
  source 1..25: mol_type = other DNA
atgccctgtattgttatttctcgtc

SEQ ID NO: 17 - DNA, 25 bp, synthetic construct
  source 1..25: mol_type = other DNA
attctaagtgagttaggatgccacg

SEQ ID NO: 18 - DNA, 22 bp, synthetic construct
  source 1..22: mol_type = other DNA
cacagctggtcagtccaatgag

SEQ ID NO: 19 - DNA, 23 bp, synthetic construct
  source 1..23: mol_type = other DNA
cagaaattctcattggttacttg

SEQ ID NO: 20 - DNA, 25 bp, synthetic construct
  source 1..25: mol_type = other DNA
cagattctggtacatctcccctaga

SEQ ID NO: 21 - DNA, 26 bp, synthetic construct
  source 1..26: mol_type = other DNA
ccaattatcagttgaaggaccatttg

SEQ ID NO: 22 - DNA, 19 bp, synthetic construct
  source 1..19: mol_type = other DNA
ccctactgtcgttcttgat

SEQ ID NO: 23 - DNA, 19 bp, synthetic construct
  source 1..19: mol_type = other DNA
ccgattacgtccctgcccc

SEQ ID NO: 24 - DNA, 29 bp, synthetic construct
  source 1..29: mol_type = other DNA
cctatcagctttagacggtagggtattgg

SEQ ID NO: 25 - DNA, 18 bp, synthetic construct
  source 1..18: mol_type = other DNA
cgacgctctccccaagga

SEQ ID NO: 26 - DNA, 20 bp, synthetic construct
  source 1..20: mol_type = other DNA
cgcaaattacccaatgaaaa

SEQ ID NO: 27 - skipped

SEQ ID NO: 28 - DNA, 21 bp, synthetic construct
  source 1..21: mol_type = other DNA
cggaatcgaaccctaattccc

SEQ ID NO: 29 - DNA, 17 bp, synthetic construct
  source 1..17: mol_type = other DNA
cggacctcgatctcgtt

SEQ ID NO: 30 - DNA, 29 bp, synthetic construct
  source 1..29: mol_type = other DNA
cggtgaaactgcgaatggctcattaaaac

SEQ ID NO: 31 - DNA, 21 bp, synthetic construct
  source 1..21: mol_type = other DNA
cgtatttaactgtcagaggtg

SEQ ID NO: 32 - DNA, 23 bp, synthetic construct
  source 1..23: mol_type = other DNA
cgtcagcaggttccacgacaaga

SEQ ID NO: 33 - DNA, 24 bp, synthetic construct
  source 1..24: mol_type = other DNA
ctcataataactttacggatcaca

SEQ ID NO: 34 - DNA, 22 bp, synthetic construct
  source 1..22: mol_type = other DNA
ctgctatgactgaggctgaata

SEQ ID NO: 35 - DNA, 20 bp, synthetic construct
  source 1..20: mol_type = other DNA
ctgtcagaggtgaaattctt

SEQ ID NO: 36 - DNA, 25 bp, synthetic construct
  source 1..25: mol_type = other DNA
cttgagacgatccagtttgtattag

SEQ ID NO: 37 - DNA, 24 bp, synthetic construct
  source 1..24: mol_type = other DNA
gcatcacagacctgttattgccta

SEQ ID NO: 38 - DNA, 21 bp, synthetic construct
  source 1..21: mol_type = other DNA
gcttgtctcaaagattaagcc

SEQ ID NO: 39 - DNA, 22 bp, synthetic construct
  source 1..22: mol_type = other DNA
ggtgagcgggcatgcatggctt

SEQ ID NO: 40 - DNA, 22 bp, synthetic construct
  source 1..22: mol_type = other DNA
ggtgcgcgggcatgcatggctt

SEQ ID NO: 41 - DNA, 22 bp, synthetic construct
  source 1..22: mol_type = other DNA
ggtgmgcgggcatgcatggctt

SEQ ID NO: 42 - DNA, 26 bp, synthetic construct
  source 1..26: mol_type = other DNA
gtacaaaatggccaattcattcaatg

SEQ ID NO: 43 - DNA, 24 bp, synthetic construct
  source 1..24: mol_type = other DNA
gtcgtggcatcctaactcacttag

SEQ ID NO: 44 - DNA, 25 bp, synthetic construct
  source 1..25: mol_type = other DNA
gtgagttaggatgccacgacaattg

SEQ ID NO: 45 - DNA, 29 bp, synthetic construct
  source 1..29: mol_type = other DNA
taaatgccctgtattgttatttctcgtca

SEQ ID NO: 46 - DNA, 25 bp, synthetic construct
  source 1..25: mol_type = other DNA
tacaaaatggccaattcattcaatg

SEQ ID NO: 47 - DNA, 28 bp, synthetic construct
  source 1..28: mol_type = other DNA
tccttggcaaatgctttcgcagtagttc

SEQ ID NO: 48 - DNA, 27 bp, synthetic construct
  source 1..27: mol_type = other DNA
tctgtcttcttagagggactttgtatg

SEQ ID NO: 49 - DNA, 22 bp, synthetic construct
  source 1..22: mol_type = other DNA
tgcctaaaacttccctgtatta

SEQ ID NO: 50 - DNA, 16 bp, synthetic construct
  source 1..16: mol_type = other DNA
tggtgccagcagccgc

SEQ ID NO: 51 - DNA, 21 bp, synthetic construct
  source 1..21: mol_type = other DNA
ttatccatgtaaaagagacca

SEQ ID NO: 52 - DNA, 28 bp, synthetic construct
  source 1..28: mol_type = other DNA
ttatctgtcttcttagagggactttgta

SEQ ID NO: 53 - DNA, 21 bp, synthetic construct
  source 1..21: mol_type = other DNA
  modified_base 5..6: mod_base = m5c
  modified_base 19..20: mod_base = m5c
ttatccatgtaaaagagacca

SEQ ID NO: 54 - DNA, 25 bp, synthetic construct
  source 1..25: mol_type = other DNA
  modified_base 3: mod_base = m5c
  modified_base 17: mod_base = m5c
tacaaaatggccaattcattcaatg

SEQ ID NO: 55 - DNA, 16 bp, synthetic construct
  source 1..16: mol_type = other DNA
  modified_base 6: mod_base = m5c
tggtgccagcagccgc

SEQ ID NO: 56 - DNA, 29 bp, synthetic construct
  source 1..29: mol_type = other DNA
  modified_base 5: mod_base = OTHER; note = propyne dU
  modified_base 7..9: mod_base = m5c
  modified_base 23: mod_base = m5c
  modified_base 25: mod_base = m5c
  modified_base 28: mod_base = m5c
taaatgccctgtattgttatttctcgtca

SEQ ID NO: 57 - DNA, 28 bp, synthetic construct
  source 1..28: mol_type = other DNA
  modified_base 2..3: mod_base = m5c
tccttggcaaatgctttcgcagtagttc

SEQ ID NO: 58 - DNA, 29 bp, synthetic construct
  source 1..29: mol_type = other DNA
  modified_base 9: mod_base = m5c
  modified_base 12: mod_base = m5c
  modified_base 19: mod_base = m5c
cggtgaaactgcgaatggctcattaaaac

SEQ ID NO: 59 - DNA, 25 bp, synthetic construct
  source 1..25: mol_type = other DNA
  modified_base 2: mod_base = OTHER; note = propyne dU
  modified_base 4..6: mod_base = m5c
  modified_base 7: mod_base = OTHER; note = propyne dU
  modified_base 12: mod_base = OTHER; note = propyne dU
  modified_base 15: mod_base = OTHER; note = propyne dU
  modified_base 18: mod_base = OTHER; note = propyne dU
  modified_base 20: mod_base = m5c
  modified_base 21: mod_base = OTHER; note = propyne dU
  modified_base 22: mod_base = m5c
atgccctgtattgttatttctcgtc

SEQ ID NO: 60-61 - skipped (2 entries)

SEQ ID NO: 62 - DNA, 22 bp, synthetic construct
  source 1..22: mol_type = other DNA
  modified_base 1: mod_base = OTHER; note = propyne dU
  modified_base 3..4: mod_base = m5c
  modified_base 5: mod_base = OTHER; note = propyne dU
  modified_base 10: mod_base = m5c
  modified_base 11: mod_base = OTHER; note = propyne dU
  modified_base 13..15: mod_base = m5c
  modified_base 18: mod_base = OTHER; note = propyne dU
  modified_base 21: mod_base = OTHER; note = propyne dU
tgcctaaaacttccctgtatta

SEQ ID NO: 63 - DNA, 22 bp, synthetic construct
  source 1..22: mol_type = other DNA
  modified_base 2: mod_base = OTHER; note = propyne dU
  modified_base 4: mod_base = m5c
  modified_base 5: mod_base = OTHER; note = propyne dU
  modified_base 7: mod_base = OTHER; note = propyne dU
  modified_base 10: mod_base = m5c
  modified_base 16: mod_base = m5c
  modified_base 17: mod_base = OTHER; note = propyne dU
  modified_base 21: mod_base = OTHER; note = propyne dU
ctgctatgactgaggctgaata

SEQ ID NO: 64 - DNA, 24 bp, synthetic construct
  source 1..24: mol_type = other DNA
  modified_base 2: mod_base = m5c
  modified_base 5: mod_base = m5c
  modified_base 7: mod_base = m5c
  modified_base 11..12: mod_base = m5c
gcatcacagacctgttattgccta

SEQ ID NO: 65 - DNA, 27 bp, synthetic construct
  source 1..27: mol_type = other DNA
  modified_base 2: mod_base = m5c
  modified_base 3: mod_base = OTHER; note = propyne dU
  modified_base 5: mod_base = OTHER; note = propyne dU
  modified_base 6: mod_base = m5c
  modified_base 9: mod_base = m5c
  modified_base 10: mod_base = OTHER; note = propyne dU
  modified_base 19: mod_base = m5c
  modified_base 20: mod_base = OTHER; note = propyne dU
  modified_base 22: mod_base = OTHER; note = propyne dU
  modified_base 24: mod_base = OTHER; note = propyne dU
  modified_base 26: mod_base = OTHER; note = propyne dU
tctgtcttcttagagggactttgtatg

SEQ ID NO: 66 - DNA, 27 bp, synthetic construct
  misc_feature 1..27: note = Synthetic Oligonucleotide
  source 1..27: mol_type = other DNA
  modified_base 2: mod_base = m5c
  modified_base 3: mod_base = OTHER; note = propyne dU
  modified_base 5: mod_base = OTHER; note = propyne dU
  modified_base 6: mod_base = m5c
  modified_base 9: mod_base = m5c
  modified_base 10: mod_base = OTHER; note = propyne dU
  modified_base 19: mod_base = m5c
  modified_base 20: mod_base = OTHER; note = propyne dU
  modified_base 22: mod_base = OTHER; note = propyne dU
  modified_base 24: mod_base = OTHER; note = propyne dU
  modified_base 26: mod_base = OTHER; note = propyne dU
tctgtcttcttagagggactttgtatg

SEQ ID NO: 67 - DNA, 29 bp, synthetic construct
  source 1..29: mol_type = other DNA
  modified_base 2: mod_base = m5c
  modified_base 5: mod_base = OTHER; note = propyne dU
  modified_base 6: mod_base = m5c
  modified_base 9: mod_base = m5c
  modified_base 16: mod_base = m5c
  modified_base 19: mod_base = OTHER; note = propyne dU
  modified_base 26: mod_base = OTHER; note = propyne dU
cctatcagctttagacggtagggtattgg

SEQ ID NO: 68 - DNA, 28 bp, synthetic construct
  source 1..28: mol_type = other DNA
  modified_base 2: mod_base = OTHER; note = propyne dU
  modified_base 4: mod_base = OTHER; note = propyne dU
  modified_base 5: mod_base = m5c
  modified_base 8: mod_base = OTHER; note = propyne dU
  modified_base 9: mod_base = m5c
  modified_base 10: mod_base = OTHER; note = propyne dU
  modified_base 12: mod_base = m5c
  modified_base 14: mod_base = OTHER; note = propyne dU
  modified_base 22: mod_base = m5c
  modified_base 23: mod_base = OTHER; note = propyne dU
  modified_base 25: mod_base = OTHER; note = propyne dU
ttatctgtcttcttagagggactttgta

SEQ ID NO: 69 - DNA, 24 bp, synthetic construct
  source 1..24: mol_type = other DNA
  modified_base 3: mod_base = m5c
  modified_base 11: mod_base = m5c
ctcataataactttacggatcaca

SEQ ID NO: 70 - DNA, 21 bp, synthetic construct
  source 1..21: mol_type = other DNA
  modified_base 10: mod_base = m5c
  modified_base 14: mod_base = m5c
cgtatttaactgtcagaggtg

SEQ ID NO: 71 - DNA, 20 bp, synthetic construct
  source 1..20: mol_type = other DNA
  modified_base 10..11: mod_base = m5c
cgcaaattacccaatgaaaa

SEQ ID NO: 72 - DNA, 19 bp, synthetic construct
  source 1..19: mol_type = other DNA
  modified_base 3: mod_base = m5c
ccctactgtcgttcttgat

SEQ ID NO: 73 - DNA, 23 bp, synthetic construct
  source 1..23: mol_type = other DNA
  modified_base 4: mod_base = m5c
  modified_base 7: mod_base = m5c
  modified_base 13..14: mod_base = m5c
  modified_base 16: mod_base = m5c
  modified_base 19: mod_base = m5c
cgtcagcaggttccacgacaaga

SEQ ID NO: 74 - DNA, 22 bp, synthetic construct
  source 1..22: mol_type = other DNA
  modified_base 7: mod_base = m5c
  modified_base 11: mod_base = m5c
  modified_base 15: mod_base = m5c
  modified_base 20: mod_base = m5c
ggtgmgcgggcatgcatggctt

SEQ ID NO: 75 - DNA, 22 bp, synthetic construct
  source 1..22: mol_type = other DNA
  modified_base 7: mod_base = m5c
  modified_base 11: mod_base = m5c
  modified_base 15: mod_base = m5c
  modified_base 20: mod_base = m5c
ggtgcgcgggcatgcatggctt

SEQ ID NO: 76 - DNA, 22 bp, synthetic construct
  source 1..22: mol_type = other DNA
  modified_base 7: mod_base = m5c
  modified_base 11: mod_base = m5c
  modified_base 15: mod_base = m5c
  modified_base 20: mod_base = m5c
ggtgagcgggcatgcatggctt

SEQ ID NO: 77 - DNA, 23 bp, synthetic construct
  source 1..23: mol_type = other DNA
  modified_base 4: mod_base = m5c
  modified_base 7: mod_base = m5c
  modified_base 13..14: mod_base = m5c
  modified_base 16: mod_base = m5c
  modified_base 19: mod_base = m5c
cgtcagcaggttccacgacaaga

SEQ ID NO: 78 - DNA, 25 bp, synthetic construct
  source 1..25: mol_type = other DNA
  modified_base 7: mod_base = OTHER; note = propyne dU
  modified_base 12: mod_base = OTHER; note = propyne dU
  modified_base 14..15: mod_base = m5c
  modified_base 17: mod_base = m5c
  modified_base 20: mod_base = m5c
  modified_base 23..24: mod_base = OTHER; note = propyne dU
gtgagttaggatgccacgacaattg

SEQ ID NO: 79 - DNA, 25 bp, synthetic construct
  source 1..25: mol_type = other DNA
  modified_base 2..3: mod_base = OTHER; note = propyne dU
  modified_base 4: mod_base = m5c
  modified_base 5: mod_base = OTHER; note = propyne dU
  modified_base 9: mod_base = OTHER; note = propyne dU
  modified_base 13..14: mod_base = OTHER; note = propyne dU
  modified_base 19: mod_base = OTHER; note = propyne dU
  modified_base 21..22: mod_base = m5c
  modified_base 24: mod_base = m5c
attctaagtgagttaggatgccacg

SEQ ID NO: 80 - DNA, 30 bp, synthetic construct; identical to SEQ ID NO: 7
  source 1..30: mol_type = other DNA
  modified_base 2: mod_base = m5c
  modified_base 7: mod_base = OTHER; note = propyne dU
  modified_base 10..11: mod_base = m5c
  modified_base 14..15: mod_base = OTHER; note = propyne dU
  modified_base 16: mod_base = m5c
  modified_base 18..19: mod_base = OTHER; note = propyne dU
  modified_base 20: mod_base = m5c
  modified_base 23: mod_base = OTHER; note = propyne dU
  modified_base 27..28: mod_base = OTHER; note = propyne dU

SEQ ID NO: 81 - DNA, 21 bp, synthetic construct
  source 1..21: mol_type = other DNA
  modified_base 8: mod_base = m5c
  modified_base 10: mod_base = m5c
attgttactctggaaggattt

SEQ ID NO: 82 - DNA, 21 bp, synthetic construct
  source 1..21: mol_type = other DNA
  modified_base 6: mod_base = OTHER; note = propyne dU
  modified_base 8: mod_base = m5c
  modified_base 10: mod_base = m5c
  modified_base 11: mod_base = OTHER; note = propyne dU
  modified_base 20: mod_base = OTHER; note = propyne dU
attgttactctggaaggattt

SEQ ID NO: 83 - DNA, 20 bp, synthetic construct
  source 1..20: mol_type = other DNA
  modified_base 3: mod_base = m5c
  modified_base 8: mod_base = m5c
  modified_base 11: mod_base = m5c
  modified_base 14: mod_base = m5c
atctaatcgtcttcaaaccc

SEQ ID NO: 84 - DNA, 22 bp, synthetic construct
  source 1..22: mol_type = other DNA
  modified_base 3..4: mod_base = m5c
  modified_base 7: mod_base = m5c
  modified_base 8: mod_base = OTHER; note = propyne dU
  modified_base 12: mod_base = OTHER; note = propyne dU
  modified_base 16: mod_base = m5c
  modified_base 18: mod_base = OTHER; note = propyne dU
agcctgctaaatagacataaga

SEQ ID NO: 85 - DNA, 19 bp, synthetic construct
  source 1..19: mol_type = other DNA
  modified_base 4: mod_base = m5c
  modified_base 6: mod_base = m5c
aagctcagcaacatgaagg

SEQ ID NO: 86 - DNA, 23 bp, synthetic construct
  source 1..23: mol_type = other DNA
  modified_base 1: mod_base = m5c
  modified_base 7: mod_base = OTHER; note = propyne dU
  modified_base 9: mod_base = m5c
  modified_base 11: mod_base = m5c
  modified_base 13: mod_base = OTHER; note = propyne dU
  modified_base 17: mod_base = OTHER; note = propyne dU
  modified_base 20: mod_base = m5c
  modified_base 22: mod_base = OTHER; note = propyne dU
cagaaattctcattggttacttg

SEQ ID NO: 87 - DNA, 21 bp, synthetic construct
  source 1..21: mol_type = other DNA
  modified_base 5..6: mod_base = m5c
  modified_base 9: mod_base = m5c
aaatccttccagagtaacaat

SEQ ID NO: 88 - DNA, 23 bp, synthetic construct
  source 1..23: mol_type = other DNA
  modified_base 2: mod_base = m5c
actgcaacaactctaatatacac

SEQ ID NO: 89 - DNA, 21 bp, synthetic construct
  source 1..21: mol_type = other DNA
  modified_base 2..3: mod_base = m5c
  modified_base 6: mod_base = m5c
  modified_base 7: mod_base = OTHER; note = propyne dU
  modified_base 11: mod_base = OTHER; note = propyne dU
  modified_base 15: mod_base = m5c
  modified_base 17: mod_base = OTHER; note = propyne dU
acctgctaaatagacataaga

SEQ ID NO: 90 - DNA, 20 bp, synthetic construct
  source 1..20: mol_type = other DNA
  modified_base 1: mod_base = m5c
  modified_base 5: mod_base = m5c
  modified_base 18: mod_base = m5c
ctgtcagaggtgaaattctt

SEQ ID NO: 91 - DNA, 21 bp, synthetic construct
  source 1..21: mol_type = other DNA
  modified_base 1: mod_base = m5c
  modified_base 7: mod_base = m5c
  modified_base 11..13: mod_base = m5c
cggaatcgaaccctaattccc

SEQ ID NO: 92 - DNA, 18 bp, synthetic construct
  source 1..18: mol_type = other DNA
  modified_base 1: mod_base = m5c
  modified_base 4: mod_base = m5c
  modified_base 6: mod_base = m5c
  modified_base 8: mod_base = m5c
cgacgctctccccaagga

SEQ ID NO: 93 - DNA, 20 bp, synthetic construct
  source 1..20: mol_type = other DNA
  modified_base 1: mod_base = m5c
  modified_base 3: mod_base = m5c
  modified_base 7..8: mod_base = OTHER; note = propyne dU
  modified_base 10..12: mod_base = m5c
  modified_base 15: mod_base = OTHER; note = propyne dU
cgcaaattacccaatgaaaa

(19) United States
(12) Patent Application Publication
(10) Pub. No.: US 2026/0234736 A1
(43) Pub. Date: Aug. 13, 2026

(54) COMPOSITIONS AND METHODS FOR DETECTING GASTROINTESTINAL PARASITES

(71) Applicant: Gen-Probe Incorporated, San Diego, CA (US)

(72) Inventors: Brian A. DOCKTER, San Diego, CA (US); Joshua K. MOBERLY, San Diego, CA (US); Brett W. KIRKCONNELL, San Diego, CA (US); Marcella A. CARVALLO PINTO, San Diego, CA (US)

(21) Appl. No.: 19/153,240

(22) PCT Filed: Mar. 15, 2024
(86) PCT No.: PCT/US2024/020126

§ 371 Date: Aug. 1, 2025

Related U.S. Application Data
(60) Provisional application No. 63/490,719, filed on Mar. 16, 2023.

(51) Int. Cl. C12Q 1/6893; C12Q 1/6806; C12Q 1/6818

(52) U.S. Cl. CPC C12Q 1/6893; C12Q 1/6806; C12Q 1/6818; C12Q 2600/16

(57) ABSTRACT

Disclosed are nucleic acid oligomers, including amplification oligomers and detection probes, for detection of Cryptosporidium spp., Entamoeba histolytica, Giardia lamblia, and Cyclospora cayetanensis target nucleic acid. Also disclosed are methods of specific nucleic acid amplification and detection using the disclosed oligomers, as well as corresponding formulations, reaction mixtures, and kits. Methods of synthesizing the nucleic acid oligomers are also disclosed.

### CROSS-REFERENCE TO RELATED APPLICATIONS

This application claims the benefit of U.S. Provisional Application No. 63/490,719, filed Mar. 16, 2023, which is incorporated by reference herein in its entirety.

### REFERENCE TO SEQUENCE LISTING

The instant application contains a Sequence Listing which has been submitted electronically in XML format and is hereby incorporated by reference in its entirety. Said XML Copy, created on Feb. 16, 2024, is named “4340_P25WO_Seq_Listing_ST26” and is 159,522 bytes in size.

### BACKGROUND

Acute diarrhea from gastrointestinal (GI) infections is the leading cause of outpatient visits, hospitalizations, and loss of quality of life, with an estimated global impact of 500 million illnesses and 230,000 deaths annually. Most GI infections from bacteria, viruses, and parasites present similar symptoms, but successful treatment is dependent on accurate pathogen identification. For identification of parasites, microscopic testing is often used, but is laborious and results in inconclusive or inaccurate diagnoses. Clinicians now rely on rapid and accurate molecular diagnostics to correctly identify the causative organism, which leads to optimal infection control and appropriate treatment. The most common parasites that are known to cause GI infections are Cryptosporidium, Entamoeba histolytica, Giardia lamblia and Cyclospora cayetanensis.

Giardiasis is the most common human intestinal parasitic disease in the U.S., with one million estimated Giardiasis cases annually (Scallan et al., Emerg. Infect. Dis. 17:7-15, 2011). Giardiasis can cause acute gastrointestinal illness presented as diarrhea. Symptoms can persist for weeks, can be mild, self-limiting, or less frequently severe illness can occur sometimes with consequent irritable bowel syndrome, chronic fatigue, postinfectious arthritis, or joint pain in adults. In children, chronic sequelae can include failure to thrive and malnutrition (Berkman et al., Lancet 359:564-571, 2002). Giardia lambia is divided into eight distinct genetic assemblages (A-H). Only assemblages A and B are known to infect humans (Heyworth, Parasite 23:13, 2016).

Cryptosporidiosis is the leading cause of U.S. waterborne disease outbreaks (Hlavsa et al., Morb. Mortal. Wkly. Rep. 67:547-551, 2018; Hlavsa et al., Morb. Mortal. Wkly. Rep. 70:733-738, 2021). An estimated 823,000 cryptosporidiosis cases occur annually in U.S (CDC, NNDSS Summary report for 2019). Cryptosporidium hominis and Cryptosporidium parvum are responsible for most human infections (Ryan et al., Parasitology, 141:1667-85, 2014; Khalil et al., Gastroenterol. Hepatol. Bed. Bench. 10:311-318, 2017). Other species like C. meleagridis, C. ubiquitum, C. felis, and C. canis are less common in humans.

Entamoeba histolytica and Cyclospora cayetanensis are parasites of lower incidence among parasitic GI infections. Although amoebiasis caused by E. histolytica is a greater concern in developing countries, the increased travel to developed countries has made this illness more common in countries like the U.S. In 2007, the California Department of Public Health reported 411 cases of amoebiasis in this state alone and estimated the prevalence of E. histolytica infection in the United States to be approximately 4% (Kantor et. al., Can. J. Gastroenterol. Hepatol., 2018:4601420, 2018). Cyclosporiasis can originate from ingesting contaminated food or water. Sporadic outbreaks in the U.S. occur. In 2018 there were 2,299 cases, across 33 states causing 160 hospitalizations. In 2021, 1,020 laboratory-confirmed cases (including 70 hospitalizations) of cyclosporiasis were reported to CDC. The current update for 2022 is 1,129 laboratory-confirmed cases with 74 hospitalizations across 33 states (2018, 2021, and 2022 reports of domestically acquired cases of cyclosporiasis, CDC).

There is a need to efficiently and sensitively detect the presence of Cryptosporidium spp., Entamoeba histolytica, Giardia lamblia, and Cyclospora cayetanensis in samples, including biological specimens to provide diagnostic and prognostic information to physicians treating patients suffering from, or suspected of suffering from, parasitic gastroenteritis or related disorders.

### SUMMARY

In some aspects, the present invention provides a composition or kit for determining the presence or absence of at least one enteric parasite in a sample, wherein the at least one enteric parasite is selected from the group consisting of Cryptosporidium spp., Entamoeba histolytica, Giardia lambia, and Cyclospora cayetanensis. In one such aspect, the composition or kit generally includes a set of oligonucleotides comprising at least one of (a) a Cryptosporidium-specific amplification oligomer set capable of amplifying a target region of a Cryptosporidium spp. target nucleic acid, (b) an Entamoeba-specific amplification oligomer set capable of amplifying a target region of an Entamoeba histolytica target nucleic acid, (c) a Giardia-specific amplification oligomer set capable of amplifying a target region of a Giardia lamblia target nucleic acid, and (d) a Cyclospora-specific amplification oligomer set capable of amplifying a target region of a Cyclospora cayetanensis target nucleic acid. In another, non-mutually exclusive aspect, the composition or kit generally includes at least one detection probe oligomer capable of hybridizing to a target region of a Cryptosporidium spp., Entamoeba histolytica, Giardia lamblia, or Cyclospora cayetanensis target nucleic acid or to an amplicon of said target region.

In another aspect, the present invention provides an oligonucleotide for determining the presence or absence of an enteric parasite selected from the group consisting of Cryptosporidium spp., Entamoeba histolytica, Giardia lamblia, and Cyclospora cayetanensis, wherein said oligonucleotide comprises a nucleotide sequence selected from the group consisting of SEQ ID NOs:1-14, 16-26, and 28-52, including from 0 to 16 nucleotide analogs.

In other aspects, the present invention provides a reaction mixture for determining the presence or absence of at least one enteric parasite in a sample, wherein the at least one enteric parasite is selected from the group consisting of Cryptosporidium spp., Entamoeba histolytica, Giardia lamblia, and Cyclospora cayetanensis. In one such aspect, the composition or kit generally includes a set of oligonucleotides comprising at least one of (a) a Cryptosporidium-specific amplification oligomer set capable of amplifying a target region of a Cryptosporidium spp. target nucleic acid, (b) an Entamoeba-specific amplification oligomer set capable of amplifying a target region of an Entamoeba histolytica target nucleic acid, (c) a Giardia-specific amplification oligomer set capable of amplifying a target region of a Giardia lamblia target nucleic acid, and (d) a Cyclospora-specific amplification oligomer set capable of amplifying a target region of a Cyclospora cayetanensis target nucleic acid. In another, non-mutually exclusive aspect, the reaction mixture generally includes at least one detection probe oligomer capable of hybridizing to a target region of a Cryptosporidium spp., Entamoeba histolytica, Giardia lamblia, or Cyclospora cayetanensis target nucleic acid or to an amplicon of said target region. In yet another non-mutually exclusive aspect, the reaction mixture comprises an oligonucleotide comprising a nucleotide sequence selected from the group consisting of SEQ ID NOs:1-14, 16-26, and 28-52, including from 0 to 16 nucleotide analogs.

In another aspect, the present invention provides a method for determining the presence or absence of at least one enteric parasite in a sample, wherein the at least one enteric parasite is selected from the group consisting of Cryptosporidium spp., Entamoeba histolytica, Giardia lamblia, and Cyclospora cayetanensis. The method generally includes performing an in vitro nucleic acid amplification reaction, utilizing an oligomer combination capable of amplifying a target region of Cryptosporidium spp., Entamoeba histolytica, Giardia lamblia, and/or Cyclospora cayetanensis target nucleic acid, to generate one or more amplification products corresponding to the Cryptosporidium spp., Entamoeba histolytica, Giardia lamblia, and/or Cyclospora cayetanensis target region, and detecting the presence or absence of the one or more amplification products.

In another aspect, the present invention provides a method for synthesizing an oligonucleotide, wherein the oligonucleotide comprises a nucleotide sequence selected from the group consisting of SEQ ID NOs:1-14, 16-26, and 28-52, including from 0 to 16 nucleotide analogs (e.g., a nucleotide sequence selected from the group consisting of SEQ ID NOs:53-59 and 62-93). In a related aspect, the present invention provides a method for synthesizing a pair of oligonucleotides, comprising synthesizing a first oligonucleotide and synthesizing a second oligonucleotide, wherein the first oligonucleotide and the second oligonucleotide target sequences flanking a target region of a Cryptosporidium spp., Entamoeba histolytica, Giardia lamblia, or Cyclospora cayetanensis target nucleic acid, and wherein the first oligonucleotide and the second oligonucleotide each comprise a nucleotide sequence selected from the group consisting of SEQ ID NOs:1-14, 16-26, and 28-52, including from 0 to 16 nucleotide analogs.

Representative embodiments of these aspects are further set forth below.

### EMBODIMENTS

Embodiment 1. A composition or kit for determining the presence or absence of at least one enteric parasite in a sample, wherein the at least one enteric parasite is selected from the group consisting of Cryptosporidium spp., Entamoeba histolytica, Giardia lamblia, and Cyclospora cayetanensis, said composition or kit comprising a set of oligonucleotides comprising at least one of (a)-(d):
(a) a Cryptosporidium-specific amplification oligomer set capable of amplifying a target region of a Cryptosporidium spp. target nucleic acid, wherein the Cryptosporidium-specific amplification oligomer set comprises first and second Cryptosporidium-specific amplification oligomers respectively comprising target-hybridizing sequences substantially corresponding to the nucleotide sequences of (i) SEQ ID NO:8 and SEQ ID NO:37; (ii) SEQ ID NO:33 and SEQ ID NO:28; (iii) SEQ ID NO:4 and SEQ ID NO:37; (iv) SEQ ID NO:11 and SEQ ID NO:49; (v) SEQ ID NO:4 and SEQ ID NO:49; (vi) SEQ ID NO:11 and SEQ ID NO:37; or (vii) SEQ ID NO:8 and SEQ ID NO:49;
(b) an Entamoeba-specific amplification oligomer set capable of amplifying a target region of an Entamoeba histolytica target nucleic acid, wherein the Entamoeba-specific amplification oligomer set comprises first and second Entamoeba-specific amplification oligomers respectively comprising target-hybridizing sequences substantially corresponding to the nucleotide sequences of (i) SEQ ID NO:46 and SEQ ID NO:19; (ii) SEQ ID NO:21 and SEQ ID NO:20; (iii) SEQ ID NO:36 and SEQ ID NO:43; or (iv) SEQ ID NO:42 and SEQ ID NO:19;
(c) a Giardia-specific amplification oligomer set capable of amplifying a target region of a Giardia lamblia target nucleic acid, wherein the Giardia-specific amplification oligomer set comprises first and second Giardia-specific amplification oligomers respectively comprising target-hybridizing sequences substantially corresponding to the nucleotide sequences of (i) SEQ ID NO:25 and SEQ ID NO:2; (ii) SEQ ID NO:5 and SEQ ID NO:29; (iii) SEQ ID NO:6 and SEQ ID NO:29; or (iv) SEQ ID NO:25 and SEQ ID NO:3; and
(d) a Cyclospora-specific amplification oligomer set capable of amplifying a target region of a Cyclospora cayetanensis target nucleic acid, wherein the Cyclospora-specific amplification oligomer set comprises first and second Cyclospora-specific amplification oligomers respectively comprising target-hybridizing sequences substantially corresponding to the nucleotide sequences of (i) SEQ ID NO:26 and SEQ ID NO:14; (ii) SEQ ID NO:23 and SEQ ID NO:18; (iii) SEQ ID NO:31 and SEQ ID NO:22; (iv) SEQ ID NO:35 and SEQ ID NO: 13, (v) SEQ ID NO:38 and SEQ ID NO:51; or (vii) SEQ ID NO:1 and SEQ ID NO:9.

Embodiment 2. The composition or kit of Embodiment 1, wherein the set of oligonucleotides comprises the Cryptosporidium-specific amplification oligomer set.

Embodiment 3. The composition or kit of Embodiment 2, wherein the set of oligonucleotides further comprises a Cryptosporidium-specific detection probe comprising a target-hybridizing sequence substantially corresponding to the nucleotide sequence of
SEQ ID NO:52 or SEQ ID NO:48 if the Cryptosporidium-specific amplification oligomer set comprises first and second Cryptosporidium-specific amplification oligomers respectively comprising target-hybridizing sequences substantially corresponding to the nucleotide sequences of (i) SEQ ID NO:8 and SEQ ID NO:37, (ii) SEQ ID NO:4 and SEQ ID NO:37, (iii) SEQ ID NO:11 and SEQ ID NO:49, (iv) SEQ ID NO:4 and SEQ ID NO:49, (v) SEQ ID NO:11 and SEQ ID NO:37, or (vi) SEQ ID NO:8 and SEQ ID NO:49; or
SEQ ID NO:24 if the Cryptosporidium-specific amplification oligomer set comprises first and second Cryptosporidium-specific amplification oligomers respectively comprising target-hybridizing sequences substantially corresponding to the nucleotide sequences of SEQ ID NO:33 and SEQ ID NO:28.

Embodiment 4. The composition or kit of any one of Embodiments 1 to 3, wherein the set of oligonucleotides comprises the Entamoeba-specific amplification oligomer set.

Embodiment 5. The composition or kit of Embodiment 4, wherein the set of oligonucleotides further comprises an Entamoeba-specific detection probe comprising a target-hybridizing sequence substantially corresponding to the nucleotide sequence of
SEQ ID NO:17 if the Entamoeba-specific amplification oligomer set comprises first and second Entamoeba-specific amplification oligomers respectively comprising target-hybridizing sequences substantially corresponding to the nucleotide sequences of SEQ ID NO:46 and SEQ ID NO:19;
SEQ ID NO:34 if the Entamoeba-specific amplification oligomer set comprises first and second Entamoeba-specific amplification oligomers respectively comprising target-hybridizing sequences substantially corresponding to the nucleotide sequences of SEQ ID NO:21 and SEQ ID NO:20;
SEQ ID NO:7 if the Entamoeba-specific amplification oligomer set comprises first and second Entamoeba-specific amplification oligomers respectively comprising target-hybridizing sequences substantially corresponding to the nucleotide sequences of SEQ ID NO:36 and SEQ ID NO:43; or
SEQ ID NO:44 if the Entamoeba-specific amplification oligomer set comprises first and second Entamoeba-specific amplification oligomers respectively comprising target-hybridizing sequences substantially corresponding to the nucleotide sequences of SEQ ID NO:42 and SEQ ID NO:19.

Embodiment 6. The composition or kit of any one of Embodiments 1 to 5, wherein the set of oligonucleotides comprises the Giardia-specific amplification oligomer set.

Embodiment 7. The composition or kit of Embodiment 6, wherein the set of oligonucleotides further comprises a Giardia-specific detection probe comprising a target-hybridizing sequence substantially corresponding to the nucleotide sequence of
SEQ ID NO:39, SEQ ID NO:40, or SEQ ID NO:41 if the Giardia-specific amplification oligomer set comprises first and second Giardia-specific amplification oligomers respectively comprising target-hybridizing sequences substantially corresponding to the nucleotide sequences of (i) SEQ ID NO:25 and SEQ ID NO:2 or (ii) SEQ ID NO:25 and SEQ ID NO:3; or
SEQ ID NO:32 if the Giardia-specific amplification oligomer set comprises first and second Giardia-specific amplification oligomers respectively comprising target-hybridizing sequences substantially corresponding to the nucleotide sequences of (i) SEQ ID NO:5 and SEQ ID NO:29 or (ii) SEQ ID NO:6 and SEQ ID NO:29.

Embodiment 8. The composition or kit of any one of Embodiments 1 to 7, wherein the set of oligonucleotides comprises the Cyclospora-specific amplification oligomer set.

Embodiment 9. The composition or kit of Embodiment 8, wherein the set of oligonucleotides further comprises a Cyclospora-specific detection probe comprising a target-hybridizing sequence substantially corresponding to the nucleotide sequence of
SEQ ID NO:16 or SEQ ID NO:45 if the Cyclospora-specific amplification oligomer set comprises first and second Cyclospora-specific amplification oligomers respectively comprising target-hybridizing sequences substantially corresponding to the nucleotide sequences of SEQ ID NO:26 and SEQ ID NO:14;
SEQ ID NO:12 if the Cyclospora-specific amplification oligomer set comprises first and second Cyclospora-specific amplification oligomers respectively comprising target-hybridizing sequences substantially corresponding to the nucleotide sequences of SEQ ID NO:23 and SEQ ID NO:18;
SEQ ID NO:10 if the Cyclospora-specific amplification oligomer set comprises first and second Cyclospora-specific amplification oligomers respectively comprising target-hybridizing sequences substantially corresponding to the nucleotide sequences of SEQ ID NO:31 and SEQ ID NO:22;
SEQ ID NO:47 if the Cyclospora-specific amplification oligomer set comprises first and second Cyclospora-specific amplification oligomers respectively comprising target-hybridizing sequences substantially corresponding to the nucleotide sequences of SEQ ID NO:35 and SEQ ID NO:13;
SEQ ID NO:30 if the Cyclospora-specific amplification oligomer set comprises first and second Cyclospora-specific amplification oligomers respectively comprising target-hybridizing sequences substantially corresponding to the nucleotide sequences of SEQ ID NO:38 and SEQ ID NO:51; or
SEQ ID NO:50 if the Cyclospora-specific amplification oligomer set comprises first and second Cyclospora-specific amplification oligomers respectively comprising target-hybridizing sequences substantially corresponding to the nucleotide sequences of SEQ ID NO:1 and SEQ ID NO:9.

Embodiment 10. The composition or kit of any one of Embodiments 1 to 9, wherein the set of oligonucleotides comprises at least two of the Cryptosporidium-specific amplification oligomer set, the Entamoeba-specific amplification oligomer set, the Giardia-specific amplification oligomer set, and the Cyclospora-specific amplification oligomer set.

Embodiment 11. The composition or kit of any one of Embodiments 1 to 9, wherein the set of oligonucleotides comprises at least three of the Cryptosporidium-specific amplification oligomer set, the Entamoeba-specific amplification oligomer set, the Giardia-specific amplification oligomer set, and the Cyclospora-specific amplification oligomer set.

Embodiment 12. The composition or kit of claim 1, wherein the set of oligonucleotides comprises the Cryptosporidium-specific amplification oligomer set, the Entamoeba-specific amplification oligomer set, the Giardia-specific amplification oligomer set, and the Cyclospora-specific amplification oligomer set.

Embodiment 13. The composition or kit of Embodiment 12, wherein the Cryptosporidium-specific amplification oligomer set comprises first and second Cryptosporidium-specific amplification oligomers respectively comprising target-hybridizing sequences substantially corresponding to the nucleotide sequences of SEQ ID NO:8 and SEQ ID NO:37.

Embodiment 14. The composition or kit of Embodiment 13, wherein the first Cryptosporidium-specific amplification oligomer comprises a target-hybridizing sequence as shown in SEQ ID NO:8, including from 0 to 16 nucleotide analogues; and/or the second Cryptosporidium-specific amplification oligomer comprises a target-hybridizing sequence as shown in SEQ ID NO:37, including from 0 to 16 nucleotide analogs.

Embodiment 15. The composition or kit of Embodiment 13 or 14, wherein the set of oligonucleotides further comprises a Cryptosporidium-specific detection probe comprising a target-hybridizing sequence as shown in SEQ ID NO:52 or SEQ ID NO:48, including from 0 to 16 nucleotide analogs.

Embodiment 16. The composition or kit of any one of Embodiments 12 to 15, wherein the Entamoeba-specific amplification oligomer set comprises first and second Entamoeba-specific amplification oligomers respectively comprising target-hybridizing sequences substantially corresponding to the nucleotide sequences of SEQ ID NO:46 and SEQ ID NO:19.

Embodiment 17. The composition or kit of Embodiment 16, wherein the first Entamoeba-specific amplification oligomer comprises a target-hybridizing sequence as shown in SEQ ID NO:46, including from 0 to 16 nucleotide analogues; and/or the second Entamoeba-specific amplification oligomer comprises a target-hybridizing sequence as shown in SEQ ID NO:19, including from 0 to 16 nucleotide analogs.

Embodiment 18. The composition or kit of Embodiment 16 or 17, wherein the set of oligonucleotides further comprises an Entamoeba-specific detection probe comprising a target-hybridizing sequence as shown in SEQ ID NO:17, including from 0 to 16 nucleotide analogs.

Embodiment 19. The composition or kit of any one of Embodiments 12 to 18, wherein the Giardia-specific amplification oligomer set comprises first and second Giardia-specific amplification oligomers respectively comprising target-hybridizing sequences substantially corresponding to the nucleotide sequences of SEQ ID NO:25 and SEQ ID NO:2.

Embodiment 20. The composition or kit of Embodiment 19, wherein the first Giardia-specific amplification oligomer comprises a target-hybridizing sequence as shown in SEQ ID NO:25, including from 0 to 16 nucleotide analogues; and/or the second Giardia-specific amplification oligomer comprises a target-hybridizing sequence as shown in SEQ ID NO:2, including from 0 to 16 nucleotide analogs.

Embodiment 21. The composition or kit of Embodiment 19 or 20, wherein the set of oligonucleotides further comprises a Giardia-specific detection probe comprising a target-hybridizing sequence as shown in SEQ ID NO:39, SEQ ID NO:40, or SEQ ID NO:41, including from 0 to 16 nucleotide analogs.

Embodiment 22. The composition or kit of any one of Embodiments 12 to 21, wherein the Cyclospora-specific amplification oligomer set comprises first and second Cyclospora-specific amplification oligomers respectively comprising target-hybridizing sequences substantially corresponding to the nucleotide sequences of SEQ ID NO:26 and SEQ ID NO:14.

Embodiment 23. The composition or kit of Embodiment 22, wherein the first Cyclospora-specific amplification oligomer comprises a target-hybridizing sequence as shown in SEQ ID NO:26, including from 0 to 16 nucleotide analogues; and/or the second Cyclospora-specific amplification oligomer comprises a target-hybridizing sequence as shown in SEQ ID NO:14, including from 0 to 16 nucleotide analogs.

Embodiment 24. The composition or kit of Embodiment 22 or 23, wherein the set of oligonucleotides further comprises a Cyclospora-specific detection probe comprising a target-hybridizing sequence as shown in SEQ ID NO:16 or SEQ ID NO:45, including from 0 to 16 nucleotide analogs.

Embodiment 25. The composition or kit of any one of Embodiments 15, 18, 21, and 24, wherein one or more of the detection probes comprises a detectable label.

Embodiment 26. The composition or kit of Embodiment 25, wherein the detectable label is a fluorescent or chemiluminescent label.

Embodiment 27. The composition or kit of Embodiment 25, wherein the detectable label is a fluorescent label and each of the one or more detection probes further comprises a non-fluorescent quencher.

Embodiment 28. The composition or kit of any one of Embodiments 1 to 27, wherein the set of oligonucleotides are contained in a formulation comprising at least one of (a) a non-linear surfactant, (b) a lyoprotectant, and (c) a chelating agent.

Embodiment 29. The composition or kit of Embodiment 28, wherein the formulation is a lyophilized formulation.

Embodiment 30. An oligonucleotide for determining the presence or absence of an enteric parasite, wherein said oligonucleotide comprises a nucleotide sequence selected from the group consisting of SEQ ID NOs:1-14, 16-26, and 28-52, including from 0 to 16 nucleotide analogs.

Embodiment 31. The oligonucleotide of Embodiment 30, wherein the nucleotide sequence is selected from the group consisting of SEQ ID NOs:53-59 and 62-93.

Embodiment 32. The oligonucleotide of Embodiment 30 or 31, wherein the 3′ end of said oligonucleotide is attached to a solid support.

Embodiment 33. The oligonucleotide of Embodiment 32, wherein the solid support is a controlled pore glass.

Embodiment 34. A reaction mixture for determining the presence or absence of at least one enteric parasite in a sample, said reaction mixture comprising a set of oligonucleotides as specified in any one of Embodiments 1 to 27.

Embodiment 35. A reaction mixture for determining the presence or absence of an enteric parasite in a sample, said reaction mixture comprising the oligonucleotide of Embodiment 30 or 31.

Embodiment 36. The reaction mixture of Embodiment 34 or 35, further comprising at least one of (a) a non-linear surfactant, (b) a lyoprotectant, (c) α-cyclodextrin, and (d) a chelating agent.

Embodiment 37. A method for determining the presence or absence of at least one enteric parasite in a sample, wherein the at least one enteric parasite is selected from the group consisting of Cryptosporidium spp., Entamoeba histolytica, Giardia lambia, and Cyclospora cayetanensis, the method comprising:
(1) contacting a sample, said sample suspected of containing the at least one enteric parasite, with an oligomer combination capable of amplifying a target region of Cryptosporidium spp., Entamoeba histolytica, Giardia lamblia, and Cyclospora cayetanensis target nucleic acid, said oligomer combination comprising
   (a) a Cryptosporidium-specific amplification oligomer set capable of amplifying a target region of a Cryptosporidium spp. target nucleic acid, wherein the Cryptosporidium-specific amplification oligomer set comprises first and second Cryptosporidium-specific amplification oligomers respectively comprising target-hybridizing sequences substantially corresponding to the nucleotide sequences of (i) SEQ ID NO:8 and SEQ ID NO:37; (ii) SEQ ID NO:33 and SEQ ID NO:28; (iii) SEQ ID NO:4 and SEQ ID NO:37; (iv) SEQ ID NO:11 and SEQ ID NO:49; (v) SEQ ID NO:4 and SEQ ID NO:49; (vi) SEQ ID NO: 11 and SEQ ID NO:37: or (vii) SEQ ID NO:8 and SEQ ID NO:49; and/or
   (b) an Entamoeba-specific amplification oligomer set capable of amplifying a target region of an Entamoeba histolytic target nucleic acid, wherein the Entamoeba-specific amplification oligomer set comprises first and second Entamoeba-specific amplification oligomers respectively comprising target-hybridizing sequences substantially corresponding to the nucleotide sequences of (i) SEQ ID NO:46 and SEQ ID NO:19; (ii) SEQ ID NO:21 and SEQ ID NO:20; (iii) SEQ ID NO:36 and SEQ ID NO:43; or (iv) SEQ ID NO:42 and SEQ ID NO:19; and/or
   (c) a Giardia-specific amplification oligomer set capable of amplifying a target region of a Giardia lamblia target nucleic acid, wherein the Giardia-specific amplification oligomer set comprises first and second Giardia-specific amplification oligomers respectively comprising target-hybridizing sequences substantially corresponding to the nucleotide sequences of (i) SEQ ID NO:25 and SEQ ID NO:2; (ii) SEQ ID NO:5 and SEQ ID NO:29; (iii) SEQ ID NO:6 and SEQ ID NO:29; or (iv) SEQ ID NO:25 and SEQ ID NO:3; and/or
   (d) a Cyclospora-specific amplification oligomer set capable of amplifying a target region of a Cyclospora cayetanensis target nucleic acid, wherein the Cyclospora-specific amplification oligomer set comprises first and second Cyclospora-specific amplification oligomers respectively comprising target-hybridizing sequences substantially corresponding to the nucleotide sequences of (i) SEQ ID NO:26 and SEQ ID NO:14; (ii) SEQ ID NO:23 and SEQ ID NO: 18; (iii) SEQ ID NO:31 and SEQ ID NO:22; (iv) SEQ ID NO:35 and SEQ ID NO:13; (v) SEQ ID NO:38 and SEQ ID NO:51; or (vii) SEQ ID NO:1 and SEQ ID NO:9;
(2) performing an in vitro nucleic acid amplification reaction, wherein any Cryptosporidium spp., Entamoeba histolytica, Giardia lamblia, and/or Cyclospora cayetanensis target nucleic acid present in the sample is used as a template for generating one or more amplification products corresponding to the Cryptosporidium spp., Entamoeba histolytica, Giardia lamblia, and/or Cyclospora cayetanensis target regions; and
(3) detecting the presence or absence of the one or more amplification products, thereby determining the presence or absence of the at least one enteric parasite in the sample.

Embodiment 38. The method of Embodiment 37, wherein the sample is contacted with the Cryptosporidium-specific amplification oligomer set and any Cryptosporidium spp. target nucleic acid present in the sample is used as a template for generating a Cryptosporidium spp. amplification product corresponding to the Cryptosporidium spp. target region.

Embodiment 39. The method of Embodiment 38, wherein the detecting step (3) comprises contacting the in vitro nucleic acid amplification reaction with a Cryptosporidium-specific detection probe configured to specifically hybridize to the Cryptosporidium spp. amplification product.

Embodiment 40. The method of Embodiment 39, wherein the Cryptosporidium-specific detection probe comprises a target-hybridizing sequence substantially corresponding to the nucleotide sequence of
SEQ ID NO:52 or SEQ ID NO:48 if the Cryptosporidium-specific amplification oligomer set comprises first and second Cryptosporidium-specific amplification oligomers respectively comprising target-hybridizing sequences substantially corresponding to the nucleotide sequences of (i) SEQ ID NO:8 and SEQ ID NO:37, (ii) SEQ ID NO:4 and SEQ ID NO:37, (iii) SEQ ID NO:11 and SEQ ID NO:49, (iv) SEQ ID NO:4 and SEQ ID NO:49, (v) SEQ ID NO:11 and SEQ ID NO:37, or (vi) SEQ ID NO:8 and SEQ ID NO:49; or
SEQ ID NO:24 if the Cryptosporidium-specific amplification oligomer set comprises first and second Cryptosporidium-specific amplification oligomers respectively comprising target-hybridizing sequences substantially corresponding to the nucleotide sequences of SEQ ID NO:33 and SEQ ID NO:28.

Embodiment 41. The method of any one of Embodiments 37 to 40, wherein the sample is contacted with the Entamoeba-specific amplification oligomer set comprising the first and second Entamoeba-specific amplification oligomers and any Entamoeba histolytica target nucleic acid present in the sample is used as a template for generating an Entamoeba histolytica amplification product corresponding to the Entamoeba histolytica target region.

Embodiment 42. The method of Embodiment 41, wherein the detecting step (3) comprises contacting the in vitro nucleic acid amplification reaction with an Entamoeba-specific detection probe configured to specifically hybridize to the Entamoeba histolytica amplification product.

Embodiment 43. The method of Embodiment 42, wherein the Entamoeba-specific detection probe comprises a target-hybridizing sequence substantially corresponding to the nucleotide sequence of
SEQ ID NO:17 if the Entamoeba-specific amplification oligomer set comprises first and second Entamoeba-specific amplification oligomers respectively comprising target-hybridizing sequences substantially corresponding to the nucleotide sequences of SEQ ID NO:46 and SEQ ID NO:19;
SEQ ID NO:34 if the Entamoeba-specific amplification oligomer set comprises first and second Entamoeba-specific amplification oligomers respectively comprising target-hybridizing sequences substantially corresponding to the nucleotide sequences of SEQ ID NO:21 and SEQ ID NO:20;
SEQ ID NO:7 if the Entamoeba-specific amplification oligomer set comprises first and second Entamoeba-specific amplification oligomers respectively comprising target-hybridizing sequences substantially corresponding to the nucleotide sequences of SEQ ID NO:36 and SEQ ID NO:43: or
SEQ ID NO:44 if the Entamoeba-specific amplification oligomer set comprises first and second Entamoeba-specific amplification oligomers respectively comprising target-hybridizing sequences substantially corresponding to the nucleotide sequences of SEQ ID NO:42 and SEQ ID NO:19.

Embodiment 44. The method of any one of Embodiments 37 to 43, wherein the sample is contacted with the Giardia-specific amplification oligomer set and any Giardia lamblia target nucleic acid present in the sample is used as a template for generating a Giardia lamblia amplification product corresponding to the Giardia lamblia target region.

Embodiment 45. The method of Embodiment 44, wherein the detecting step (3) comprises contacting the in vitro nucleic acid amplification reaction with a Giardia-specific detection probe configured to specifically hybridize to the Giardia lamblia amplification product.

Embodiment 46. The method of Embodiment 42, wherein the Giardia-specific detection probe comprises a target-hybridizing sequence substantially corresponding to the nucleotide sequence of
SEQ ID NO:39, SEQ ID NO:40, or SEQ ID NO:41 if the Giardia-specific amplification oligomer set comprises first and second Giardia-specific amplification oligomers respectively comprising target-hybridizing sequences substantially corresponding to the nucleotide sequences of (i) SEQ ID NO:25 and SEQ ID NO:2 or (ii) SEQ ID NO:25 and SEQ ID NO:3; or
SEQ ID NO:32 if the Giardia-specific amplification oligomer set comprises first and second Giardia-specific amplification oligomers respectively comprising target-hybridizing sequences substantially corresponding to the nucleotide sequences of (i) SEQ ID NO:5 and SEQ ID NO:29 or (ii) SEQ ID NO:6 and SEQ ID NO:29.

Embodiment 47. The method of any one of Embodiments 37 to 46, wherein the sample is contacted with the Cyclospora-specific amplification oligomer set and any Cyclospora cayetanensis target nucleic acid present in the sample is used as a template for generating a Cyclospora cayetanensis amplification product corresponding to the Cyclospora cayetanensis target region.

Embodiment 48. The method of Embodiment 47, wherein the detecting step (3) comprises contacting the in vitro nucleic acid amplification reaction with a Cyclospora-specific detection probe configured to specifically hybridize to the Cyclospora cayetanensis amplification product.

Embodiment 49. The method of Embodiment 48, wherein the Cyclospora-specific detection probe comprises a target-hybridizing sequence substantially corresponding to the nucleotide sequence of
SEQ ID NO:16 or SEQ ID NO:45 if the Cyclospora-specific amplification oligomer set comprises first and second Cyclospora-specific amplification oligomers respectively comprising target-hybridizing sequences substantially corresponding to the nucleotide sequences of SEQ ID NO:26 and SEQ ID NO:14;
SEQ ID NO:12 if the Cyclospora-specific amplification oligomer set comprises first and second Cyclospora-specific amplification oligomers respectively comprising target-hybridizing sequences substantially corresponding to the nucleotide sequences of SEQ ID NO:23 and SEQ ID NO:18;
SEQ ID NO:10 if the Cyclospora-specific amplification oligomer set comprises first and second Cyclospora-specific amplification oligomers respectively comprising target-hybridizing sequences substantially corresponding to the nucleotide sequences of SEQ ID NO:31 and SEQ ID NO:22;
SEQ ID NO:47 if the Cyclospora-specific amplification oligomer set comprises first and second Cyclospora-specific amplification oligomers respectively comprising target-hybridizing sequences substantially corresponding to the nucleotide sequences of SEQ ID NO:35 and SEQ ID NO:13;
SEQ ID NO:30 if the Cyclospora-specific amplification oligomer set comprises first and second Cyclospora-specific amplification oligomers respectively comprising target-hybridizing sequences substantially corresponding to the nucleotide sequences of SEQ ID NO:38 and SEQ ID NO:51: or
SEQ ID NO:50 if the Cyclospora-specific amplification oligomer set comprises first and second Cyclospora-specific amplification oligomers respectively comprising target-hybridizing sequences substantially corresponding to the nucleotide sequences of SEQ ID NO:1 and SEQ ID NO:9.

Embodiment 50. The method of any one of Embodiments 37 to 49, wherein the method is a multiplex method for detecting the presence of absence of at least two of Cryptosporidium spp., Entamoeba histolytica, Giardia lamblia, and Cyclospora cayetanensis.

Embodiment 51. The method of any one of Embodiments 37 to 49, wherein the method is a multiplex method for detecting the presence or absence of at least three of Cryptosporidium spp., Entamoeba histolytica, Giardia lamblia, and Cyclospora cayetanensis.

Embodiment 52. The method of any one of Embodiments 37 to 49, wherein the method is a multiplex method for detecting the presence or absence of each of Cryptosporidium spp., Entamoeba histolytica, Giardia lamblia, and Cyclospora cayetanensis.

Embodiment 53. The method of Embodiment 52, wherein the Cryptosporidium-specific amplification oligomer set comprises first and second Cryptosporidium-specific amplification oligomers respectively comprising target-hybridizing sequences substantially corresponding to the nucleotide sequences of SEQ ID NO:8 and SEQ ID NO:37.

Embodiment 54. The method of Embodiment 53, wherein the first Cryptosporidium-specific amplification oligomer comprises a target-hybridizing sequence as shown in SEQ ID NO:8, including from 0 to 16 nucleotide analogues; and/or the second Cryptosporidium-specific amplification oligomer comprises a target-hybridizing sequence as shown in SEQ ID NO:37, including from 0 to 16 nucleotide analogs.

Embodiment 55. The method of Embodiment 53 or 54, wherein the detecting step (3) comprises contacting the in vitro nucleic acid amplification reaction with a Cryptosporidium-specific detection probe comprising a target-hybridizing sequence as shown in SEQ ID NO:52 or SEQ ID NO:48, including from 0 to 16 nucleotide analogs.

Embodiment 56. The method of any one of Embodiments 52 to 55, wherein the Entamoeba-specific amplification oligomer set comprises first and second Entamoeba-specific amplification oligomers respectively comprising target-hybridizing sequences substantially corresponding to the nucleotide sequences of SEQ ID NO:46 and SEQ ID NO:19.

Embodiment 57. The method of Embodiment 56, wherein the first Entamoeba-specific amplification oligomer comprises a target-hybridizing sequence as shown in SEQ ID NO:46, including from 0 to 16 nucleotide analogues; and/or the second Entamoeba-specific amplification oligomer comprises a target-hybridizing sequence as shown in SEQ ID NO:19, including from 0 to 16 nucleotide analogs.

Embodiment 58. The method of Embodiment 56 or 57, wherein the detecting step (3) comprises contacting the in vitro nucleic acid amplification reaction with an Entamoeba-specific detection probe comprising a target-hybridizing sequence as shown in SEQ ID NO:17, including from 0 to 16 nucleotide analogs.

Embodiment 59. The method of any one of Embodiments 52 to 58, wherein the Giardia-specific amplification oligomer set comprises first and second Giardia-specific amplification oligomers respectively comprising target-hybridizing sequences substantially corresponding to the nucleotide sequences of SEQ ID NO:25 and SEQ ID NO:2.

Embodiment 60. The method of Embodiment 59, wherein the first Giardia-specific amplification oligomer comprises a target-hybridizing sequence as shown in SEQ ID NO:25, including from 0 to 16 nucleotide analogues; and/or the second Giardia-specific amplification oligomer comprises a target-hybridizing sequence as shown in SEQ ID NO:2, including from 0 to 16 nucleotide analogs.

Embodiment 61. The method of Embodiment 59 or 60, wherein the detecting step (3) comprises contacting the in vitro nucleic acid amplification reaction with a Giardia-specific detection probe comprising a target-hybridizing sequence as shown in SEQ ID NO:39, SEQ ID NO:40, or SEQ ID NO:41, including from 0 to 16 nucleotide analogs.

Embodiment 62. The method of any one of Embodiments 52 to 61, wherein the Cyclospora-specific amplification oligomer set comprises first and second Cyclospora-specific amplification oligomers respectively comprising target-hybridizing sequences substantially corresponding to the nucleotide sequences of SEQ ID NO:26 and SEQ ID NO:14.

Embodiment 63. The method of Embodiment 62, wherein the first Cyclospora-specific amplification oligomer comprises a target-hybridizing sequence as shown in SEQ ID NO:26, including from 0 to 16 nucleotide analogues; and/or the second Cyclospora-specific amplification oligomer comprises a target-hybridizing sequence as shown in SEQ ID NO:14, including from 0 to 16 nucleotide analogs.

Embodiment 64. The method of Embodiment 62 or 63, wherein the detecting step (3) comprises contacting the in vitro nucleic acid amplification reaction with a Cyclospora-specific detection probe comprising a target-hybridizing sequence as shown in SEQ ID NO:16 or SEQ ID NO:45, including from 0 to 16 nucleotide analogs.

Embodiment 65. The method of any one of Embodiments 39, 40, 42, 43, 45, 46, 48, 49, 55, 58, 61, and 64, wherein one or more of the detection probes comprises a detectable label.

Embodiment 66. The method of Embodiment 65, wherein the detectable label is a fluorescent or chemiluminescent label.

Embodiment 67. The method of Embodiment 65, wherein the detectable label is a fluorescent label and each of the one or more detection probes further comprises a non-fluorescent quencher.

Embodiment 68. The method of any one of Embodiments 37 to 67, wherein the sample is a human sample.

Embodiment 69. The method of any one of Embodiments 37 to 68, wherein the sample is a stool sample or a blood sample.

A method for synthesizing an oligonucleotide, comprising the steps of:
(a) obtaining a solid support comprising at least one nucleobase residue, wherein the at least one nucleobase residue is covalently bound at a 3′ position to the solid support;
(b) coupling a 5′ position of the nucleobase residue furthest from the solid support to a 3′ position of another nucleobase residue;
(c) repeating step (b) at least 14 additional times, thereby generating at least 16 contiguous nucleobase residues coupled to the solid support, and
(d) cleaving the at least 16 contiguous nucleobase residues generated in step (c), thereby obtaining the oligonucleotide,
wherein the oligonucleotide comprises a nucleotide sequence selected from the group consisting of SEQ ID NOs:1-14, 16-26, and 28-52, including from 0 to 16 nucleotide analogs.

Embodiment 70. The method of Embodiment 69, wherein the nucleotide sequence is selected from the group consisting of SEQ ID NOs:53-59 and 62-93.

Embodiment 71. A method for synthesizing a pair of oligonucleotides, comprising synthesizing a first oligonucleotide and synthesizing a second oligonucleotide,
wherein each of the synthesizing the first oligonucleotide and the synthesizing the second oligonucleotide comprises the steps of:
(a) obtaining a solid support comprising at least one nucleobase residue, wherein the at least one nucleobase residue is covalently bound at a 3′ position to the solid support;
(b) coupling a 5′ position of the nucleobase residue furthest from the solid support to a 3′ position of another nucleobase residue;
(c) repeating step (b) at least 15 additional times, thereby generating at least 17 contiguous nucleobase residues coupled to the solid support, and
(d) cleaving the at least 17 contiguous nucleobase residues generated in step (c), thereby obtaining the oligonucleotide, and
wherein the first oligonucleotide and the second oligonucleotide respectively comprise the nucleotide sequences of any one of
   SEQ ID NO:8 and SEQ ID NO:37, including from 0 to 16 nucleotide analogs;
   SEQ ID NO:33 and SEQ ID NO:28, including from 0 to 16 nucleotide analogs;
   SEQ ID NO:4 and SEQ ID NO:37, including from 0 to 16 nucleotide analogs;
   SEQ ID NO:11 and SEQ ID NO:49, including from 0 to 16 nucleotide analogs;
   SEQ ID NO:4 and SEQ ID NO:49, including from 0 to 16 nucleotide analogs;
   SEQ ID NO:11 and SEQ ID NO:37, including from 0 to 16 nucleotide analogs;
   SEQ ID NO:8 and SEQ ID NO:49, including from 0 to 16 nucleotide analogs;
   SEQ ID NO:46 and SEQ ID NO:19, including from 0 to 16 nucleotide analogs;
   SEQ ID NO:21 and SEQ ID NO:20, including from 0 to 16 nucleotide analogs;
   SEQ ID NO:36 and SEQ ID NO:43, including from 0 to 16 nucleotide analogs;
   SEQ ID NO:42 and SEQ ID NO:19, including from 0 to 16 nucleotide analogs;
   SEQ ID NO:25 and SEQ ID NO:2, including from 0 to 16 nucleotide analogs;
   SEQ ID NO:5 and SEQ ID NO:29, including from 0 to 16 nucleotide analogs;
   SEQ ID NO:6 and SEQ ID NO:29, including from 0 to 16 nucleotide analogs;
   SEQ ID NO:25 and SEQ ID NO:3, including from 0 to 16 nucleotide analogs;
   SEQ ID NO:26 and SEQ ID NO:14, including from 0 to 16 nucleotide analogs;
   SEQ ID NO:23 and SEQ ID NO:18, including from 0 to 16 nucleotide analogs;
   SEQ ID NO:31 and SEQ ID NO:22, including from 0 to 16 nucleotide analogs;
   SEQ ID NO:35 and SEQ ID NO:13, including from 0 to 16 nucleotide analogs;
   SEQ ID NO:38 and SEQ ID NO:51, including from 0 to 16 nucleotide analogs; or
   SEQ ID NO:1 and SEQ ID NO:9, including from 0 to 16 nucleotide analogs.

These and other aspects and embodiments will become evident upon reference to the following detailed description and the attached drawings.

### Definitions

Unless defined otherwise, all technical and scientific terms used herein have the same meaning as commonly understood by one of ordinary skill in the art pertinent to the methods and compositions described. As used herein, the following terms and phrases have the meanings ascribed to them unless specified otherwise.

The terms “a,” “an,” and “the” include plural referents, unless the context clearly indicates otherwise. For example, “a nucleic acid” as used herein is understood to represent one or more nucleic acids. As such, the terms “a” (or “an”), “one or more,” and “at least one” can be used interchangeably herein.

When a value is expressed as “about” X or “approximately” X, the stated value of X will be understood to be accurate to ±10%.

All ranges are to be interpreted as encompassing the endpoints in the absence of express exclusions such as “not including the endpoints”; thus, for example, “from 0 to 16” includes the values 0 and 16.

“Sample” includes any specimen that may contain Cryptosporidium spp., Entamoeba histolytica, Giardia lamblia, and/or Cyclospora cayetanensis, including components thereof, such as nucleic acids or fragments of nucleic acids. Samples include “biological samples” which include any tissue or material derived from a living or dead human, including, for example, stool, blood, plasma, serum, blood cells, saliva, mucous, and cerebrospinal fluid. The biological sample may be treated to physically or mechanically disrupt tissue or cell structure, thus releasing intracellular components into a solution which may further contain enzymes, buffers, salts, detergents, and the like, which are used to prepare a biological sample for analysis. Also, samples may include processed samples such as samples in which one or more components have been concentrated or purified. Processed samples include, e.g., those obtained from passing samples over or through a filtering device, or following centrifugation, or by adherence to a medium, matrix, or support.

A “nucleotide” as used herein is a subunit of a nucleic acid consisting of a phosphate group, a 5-carbon sugar, and a nitrogenous base (also referred to herein as “nucleobase”). The 5-carbon sugar found in RNA is ribose. In DNA, the 5-carbon sugar is 2′-deoxyribose.

“Nucleic acid” and “polynucleotide” refer to a multimeric compound comprising nucleotides and/or nucleotide analogs linked together to form a biopolymer. The biopolymers include conventional RNA, conventional DNA, mixed RNA-DNA, and nucleotide-analog-containing versions thereof. A nucleic acid “backbone” may be made up of a variety of linkages, including one or more of sugar-phosphodiester linkages, peptide-nucleic acid bonds (“peptide nucleic acids” or PNA), phosphorothioate linkages, methylphosphonate linkages, or combinations thereof. Sugar moieties of a nucleic acid may be ribose, deoxyribose, or similar compounds with substitutions, e.g., analogs with a methoxy, fluoro or halide group at the 2′ position of the ribose (also referred to herein as “2′-O-Me” or “2′-methoxy” or 2′-fluoro, or “2′-halide”). Nitrogenous bases may be conventional bases, adenine (A), uracil (U), guanine (G), thymine (T), and cytosine (C), and analogs thereof (e.g., inosine, 5 methyl 2′ deoxycytosine (“5-methyl cytosine”) (5mC), isoguanine, propyne dC (pdC), or propyne dU (pdU)). As used in the present disclosure, pdC is considered a cytosine analogue, and pdU is considered a thymine analogue. Nucleic acids may include one or more “abasic” residues where the backbone includes no nitrogenous base for position(s) of the polymer.

By “RNA and DNA equivalents” is meant RNA and DNA molecules having essentially the same complementary base pair hybridization properties. RNA and DNA equivalents have different sugar moieties (i.e., ribose versus deoxyribose) and may differ by the presence of uracil in RNA and thymine in DNA. The differences between RNA and DNA equivalents do not contribute to differences in homology because the equivalents have the same degree of complementarity to a particular sequence. By “DNA/RNA chimeric” is meant a nucleic acid comprising both DNA and RNA nucleotides. One example of a DNA/RNA chimeric is a DNA oligomer wherein all thymine (T) nucleobase residues are replaced with uracil (U). Unless the context clearly dictates otherwise, reference to a Cryptosporidium spp., Entamoeba histolytica, Giardia lamblia, or Cyclospora cayetanensis nucleic acid includes the RNA and DNA equivalents and DNA/RNA chimerics thereof.

The phrase “including from 0 to 16 nucleotide analogs,” as used herein following reference to one or more nucleotide sequences by SEQ ID NO, means that the referenced sequence(s) include equivalents of each sequence having from 0 to 16 nucleotide analogs (also referred to herein as “modified nucleotides”). By “equivalents having from 0 to 16 nucleotide analogs” is meant oligonucleotides that (i) have from 0 to 16 nucleotide analogs substituting conventional nucleotides within the reference sequence and (ii) have essentially the same complementary base pair hybridization properties as the reference sequence. Exemplary modified nucleotides are shown in Table 27, infra.

“Oligomer,” “oligonucleotide,” or “oligo” refers to a nucleic acid of generally less than 1,000 nucleotides (nt), including those in a size range having a lower limit of about 5 nt and an upper limit of about 500 to 900 nt. Some particular embodiments are oligonucleotides in a size range with a lower limit of about 5 to 15, 16, 17, 18, 19, or 20 nt and an upper limit of about 50 to 600 nt, and other particular embodiments are in a size range with a lower limit of about 10 to 20 nt and an upper limit of about 22 to 100 nt. Oligonucleotides may be purified from naturally occurring sources but may be synthesized by using any well-known enzymatic or chemical method. Oligomers may be referred to by a functional name (e.g., detection probe, primer, or promoter primer) but those skilled in the art will understand that such terms refer to oligomers.

A “target nucleic acid” as used herein is a nucleic acid comprising a target sequence to be amplified. Target nucleic acids may be DNA or RNA and may be either single-stranded or double-stranded. The target nucleic acid may include other sequences besides the target sequence, which may not be amplified.

The term “target region” or “target nucleic acid region” as used herein refers to the particular nucleotide sequence of the target nucleic acid that is to be amplified and/or detected. The “target region” includes the complexing sequences to which oligonucleotides (e.g., priming oligonucleotides and/or promoter oligonucleotides) complex during an amplification processes (e.g., PCR. TMA). Unless the context clearly dictates otherwise, where the target nucleic acid is originally single-stranded, the term “target region” will also refer to the sequence complementary to the “target region” as present in the target nucleic acid, and where the target nucleic acid is originally double-stranded, the term “target region” refers to both the sense (+) and antisense (−) strands.

The term “target sequence” or “target nucleic acid sequence” as used herein refers to the particular nucleotide sequence of the target nucleic acid to which oligonucleotides (e.g., priming oligonucleotides, detection probes, or capture probes) complex during amplification and/or detection of the target nucleic acid.

“Target-hybridizing sequence” or “target-specific sequence” is used herein to refer to the portion of an oligomer that is configured to hybridize with a target nucleic acid sequence. Preferably, the target-hybridizing sequences are configured to specifically hybridize with a target nucleic acid sequence. Target-hybridizing sequences may be 100% complementary to the portion of the target sequence to which they are configured to hybridize, but not necessarily. Target-hybridizing sequences may also include inserted, deleted and/or substituted nucleotide residues relative to a target sequence.

“Non-target-specific sequence” or “non-target-hybridizing sequence” as used herein refers to a region of an oligomer sequence, wherein said region does not stably hybridize with a target sequence under standard hybridization conditions. Oligomers with non-target-specific sequences include, but are not limited to, promoter primers, promoter providers, target capture oligomers, torches, and molecular beacons.

The term “target a sequence,” as used herein in reference to a region of a Cryptosporidium spp., Entamoeba histolytica, Giardia lamblia, or Cyclospora cayetanensis nucleic acid, refers to a process whereby an oligonucleotide hybridizes to a target region in a manner that allows for amplification and detection as described herein. In one embodiment, the oligonucleotide is complementary with the targeted Cryptosporidium spp., Entamoeba histolytica, Giardia lamblia, or Cyclospora cayetanensis nucleic acid sequence and contains no mismatches. In another embodiment, the oligonucleotide is complementary but contains 1, 2, 3, 4, or 5 mismatches with the targeted Cryptosporidium spp., Entamoeba histolytica, Giardia lamblia, or Cyclospora cayetanensis nucleic acid sequence.

The term “configured to” denotes an actual arrangement of the polynucleotide sequence configuration of a referenced oligonucleotide target-hybridizing sequence. For example, amplification oligomers that are configured to generate a specified amplicon from a target nucleic acid region have polynucleotide sequences that hybridize to the target region and can be used in an amplification reaction to generate the amplicon. Also, as an example, oligonucleotides that are configured to specifically hybridize to a target region have a polynucleotide sequence that specifically hybridizes to the referenced sequence under stringent hybridization conditions.

The term “configured to specifically hybridize to” as used herein means that the target-hybridizing region of an amplification oligonucleotide, detection probe, or other oligonucleotide is designed to have a polynucleotide sequence that could target a sequence of the referenced Cryptosporidium spp., Entamoeba histolytica, Giardia lamblia, or Cyclospora cayetanensis target region. The oligonucleotide is designed to function as a component of an assay for amplification and detection of Cryptosporidium spp., Entamoeba histolytica, Giardia lamblia, or Cyclospora cayetanensis target nucleic acid from a sample, and therefore is designed to target Cryptosporidium spp., Entamoeba histolytica, Giardia lamblia, or Cyclospora cayetanensis nucleic acid in the presence of other nucleic acids commonly found in testing samples. “Specifically hybridize to” does not mean exclusively hybridize to, as some small level of hybridization to non-target nucleic acids may occur, as is understood in the art. Rather. “specifically hybridize to” means that the oligonucleotide is configured to function in an assay to primarily hybridize the target so that an accurate detection of target nucleic acid in a sample can be determined.

An “amplification oligonucleotide” or “amplification oligomer” is an oligonucleotide that hybridizes to a target nucleic acid and participates in a nucleic acid amplification reaction, e.g., serving as a primer. Amplification oligomers can have 3′ ends that are extended by polymerization as part of the nucleic acid amplification reaction. Amplification oligomers can alternatively have 3′ ends that are not extended by polymerization, but provide a component that facilitates nucleic acid amplification, e.g., a promoter sequence joined 5′ to the target hybridizing sequence of the amplification oligomer. Such an amplification oligomer is referred to as a promoter provider. Amplification oligomers that provide both a 3′ target hybridizing region that is extendable by polymerization and a 5′ promoter sequence are referred to as promoter primers. Amplification oligomers may be optionally modified to include 5′ non-target hybridizing regions such as tags, promoters (as mentioned), or other sequences used or useful for manipulating or amplifying the primer or target oligonucleotide.

“Nucleic acid amplification” refers to any in vitro procedure that produces multiple copies of a target nucleic acid sequence, or its complementary sequence, or fragments thereof (i.e., an amplified sequence containing less than the complete target nucleic acid). Examples of nucleic acid amplification procedures include transcription associated methods, such as transcription-mediated amplification (TMA), nucleic acid sequence-based amplification (NASBA) and others (e.g., U.S. Pat. Nos. 5,399,491, 5,554,516, 5,437,990, 5,130,238, 4,868,105, and 5,124,246), and polymerase chain reaction (PCR) (e.g., U.S. Pat. Nos. 4,683,195, 4,683,202, and 4,800,159).

By “amplicon” or “amplification product” is meant a nucleic acid molecule generated in a nucleic acid amplification reaction and which is derived from a target nucleic acid. An amplicon or amplification product contains a target nucleic acid region that may be of the same or opposite sense as the target nucleic acid.

As used herein, the term “relative fluorescence unit” (“RFU”) is a unit of measurement of fluorescence intensity. RFU varies with the characteristics of the detection means used for the measurement and can be used as a measurement to compare relative intensities between samples and controls.

“Detection probe oligomer,” “detection probe,” or “probe” refers to an oligomer that hybridizes specifically to a target nucleic acid region, including an amplified product, under conditions that promote nucleic acid hybridization, for detection of the target nucleic acid. Detection may either be direct (i.e., probe hybridized directly to the target) or indirect (i.e., a probe hybridized to an intermediate structure that links the probe to the target). A probe's target sequence generally refers to the specific sequence within a larger sequence which the probe hybridizes specifically. A detection probe may include target-specific sequence(s) and non-target-specific sequence(s). Such non-target-specific sequences can include sequences which will confer a desired secondary or tertiary structure, such as a hairpin structure, which can be used to facilitate detection and/or amplification.

As used herein, a nucleic acid “substantially corresponding to” a specified nucleic acid sequence, or its complement, means that the oligonucleotide is sufficiently similar to the reference nucleic acid sequence such that the oligonucleotide has similar hybridization properties to the reference nucleic acid sequence in that it would hybridize with the same target nucleic acid sequence under stringent hybridization conditions. Substantially corresponding nucleic acids vary by at least one nucleotide from the specified nucleic acid. This variation may be stated in terms of a percentage of sequence identity or complementarity between the nucleic acid and the specified nucleic acid. In some embodiments, a nucleic acid “substantially corresponding to” a reference sequence has from about 80% to 100% nucleobase sequence identity or complementarity to the reference sequence; in preferred embodiments, the percentage is from about 85% to 100%, more preferably from about 90% to 100% or from about 95% to 100%. One skilled in the art will understand that the recited ranges include all whole and rational numbers of the range (e.g., 92%, 92.377%, etc.).

By “stringent hybridization conditions.” or “stringent conditions” is meant conditions permitting an oligomer to preferentially hybridize to a target nucleic acid region and not to nucleic acid derived from a closely related non-target nucleic acid (i.e., conditions permitting an oligomer to hybridize to its target sequence to form a stable oligomer:target hybrid, but not form a sufficient number of stable oligomer:non-target hybrids, so as to allow for amplification and/or detection of target nucleic acids but not non-targeted organisms). While the definition of stringent hybridization conditions does not vary, the actual reaction environment that can be used for stringent hybridization may vary depending upon factors including the GC content and length of the oligomer, the degree of similarity between the oligomer sequence and sequences of non-target nucleic acids that may be present in the test sample, and the target sequence. Hybridization conditions include the temperature and the composition of the hybridization reagents or solutions. Stringent hybridization conditions are readily ascertained by those having ordinary skill in the art.

“Label” or “detectable label” refers to a moiety or compound joined directly or indirectly to a probe that is detected or leads to a detectable signal. Direct joining may use covalent bonds or non-covalent interactions (e.g., hydrogen bonding, hydrophobic or ionic interactions, and chelate or coordination complex formation) whereas indirect joining may use a bridging moiety or linker (e.g., via an antibody or additional oligonucleotide(s), which may amplify a detectable signal). Any detectable moiety may be used, e.g., radionuclide, ligand such as biotin or avidin, enzyme, enzyme substrate, reactive group, chromophore such as a dye or particle (e.g., latex or metal bead) that imparts a detectable color, luminescent compound (e.g., bioluminescent, phosphorescent, or chemiluminescent compound such as an acridinium ester (“AE”) compound), and fluorescent compound (i.e., fluorophore). Embodiments of fluorophores include those that absorb light in the range of about 495 to 690 nm and emit light in the range of about 520 to 705 nm, which include those known as FAM™, TET™, CAL FLUOR™ (Orange or Red), and QUASAR™ compounds. Fluorophores may be used in combination with a quencher molecule that absorbs light when in close proximity to the fluorophore to diminish background fluorescence. Such quenchers are well known in the art and include, e.g., BLACK HOLE QUENCHER™ (or BHQ™) or TAMRA™ compounds. Particular embodiments include a “homogeneous detectable label” that is detectable in a homogeneous system in which bound labeled probe in a mixture exhibits a detectable change compared to unbound labeled probe, which allows the label to be detected without physically removing hybridized from unhybridized labeled probe (e.g., U.S. Pat. Nos. 5,283,174, 5,656,207, and 5,658,737). Particular homogeneous detectable labels include chemiluminescent compounds, including acridinium ester (“AE”) compounds, such as standard AE or AE derivatives, which are well known (U.S. Pat. Nos. 5,656,207, 5,658,737, and 5,639,604). Methods of synthesizing labels, attaching labels to nucleic acid, and detecting signals from labels are well known (e.g., Sambrook et al., Molecular Cloning, A Laboratory Manual, 2nd ed. (Cold Spring Harbor Laboratory Press, Cold Spring Harbor, NY, 1989) at Ch. 10, and U.S. Pat. Nos. 5,658,737, 5,656,207, 5,547,842, 5,283,174, and 4,581,333, and EP Pat. App. 0 747 706). Particular methods of linking an AE compound to a nucleic acid are known (e.g., U.S. Pat. Nos. 5,585,481 and 5,639,604, see column 10, line 6 to column 11, line 3, and Example 8). Particular AE labeling positions are a probe's central region and near a region of A/T base pairs, at a probe's 3′ or 5′ terminus, or at or near a mismatch site with a known sequence that is the probe should not detect compared to the desired target sequence. Other detectably labeled probes include, e.g., TaqMan™ probes, molecular torches, and molecular beacons. TaqMan™ probes include a donor and acceptor label wherein fluorescence is detected upon enzymatically degrading the probe during amplification in order to release the fluorophore from the presence of the quencher. Molecular torches and beacons exist in open and closed configurations wherein the closed configuration quenches the fluorophore and the open position separates the fluorophore from the quencher to allow fluorescence. Hybridization to target opens the otherwise closed probes. Exemplary detectable labels are shown in Table 27, infra.

A “non-extendable” oligomer includes a blocking moiety at or near its 3′-terminus to prevent extension. A blocking group near the 3′ end is in some embodiments within five residues of the 3′ end and is sufficiently large to limit binding of a polymerase to the oligomer. In other embodiments, a blocking group is covalently attached to the 3′ terminus. Suitable blocking groups include, e.g., alkyl groups, non-nucleotide linkers, alkane-diol dideoxynucleotide residues, cordycepin, 3′-deoxy nucleotides, 3′-phosphorylated nucleotides, inverted nucleotides, proteins, peptides, and labels such as fluorophores or quenchers.

References, particularly in the embodiments, to “the sequence of SEQ ID NO:X” refer to the sequence of nucleotides and/or nucleotide analogs linked together to form a biopolymer. Reference to a sequence by SEQ ID NO does not connote the identity of the backbone (e.g., RNA, 2′-O-Me RNA, or DNA) or any nucleobase modifications (e.g., methylation of cytosine residues (“5MeC”)) unless the context clearly dictates otherwise. In some instances, the sequence of a SEQ ID NO is followed by the statement “including from [x-y] nucleotide analogs”; it is understood that the nucleotide analogs may be substitutions within the sequence of the SEQ ID NO. Unless the context clearly dictates otherwise, reference to a sequence by SEQ ID NO includes reference to its complementary sequence (e.g., reference to the sequence 5′-ttagc-3′ includes reference to the sequence 5′-gctaa-3′).

“Separating” or “purifying” means that one or more components of a sample are removed or separated from other sample components. Sample components include target nucleic acids usually in a generally aqueous solution phase, which may also include cellular fragments, proteins, carbohydrates, lipids, and other nucleic acids. “Separating” or “purifying” does not connote any degree of purification. Typically, separating or purifying removes at least 70%, or at least 80%, or at least 95% of the target nucleic acid from other sample components.

The term “non-linear surfactant,” as used herein, means a surfactant having a branched chain structure. A non-linear surfactant may include one or more ring structures, which may be, for example, in a principal chain and/or in one or more branched chains. Exemplary non-linear surfactants include polysorbate 20, polysorbate 40, polysorbate 60, and digitonin. In certain variations, the non-linear surfactant is non-ionic.

The term “specificity,” in the context of an amplification and/or detection system, is used herein to refer to the characteristic of the system which describes its ability to distinguish between target and non-target sequences dependent on sequence and assay conditions. In terms of nucleic acid amplification, specificity generally refers to the ratio of the number of specific amplicons produced to the number of side-products (e.g., the signal-to-noise ratio). In terms of detection, specificity generally refers to the ratio of signal produced from target nucleic acids to signal produced from non-target nucleic acids.

The term “sensitivity” is used herein to refer to the precision with which a nucleic acid amplification reaction can be detected or quantitated. The sensitivity of an amplification reaction is generally a measure of the smallest copy number of the target nucleic acid that can be reliably detected in the amplification system, and will depend, for example, on the detection assay being employed, and the specificity of the amplification reaction, e.g., the ratio of specific amplicons to side-products.

### DETAILED DESCRIPTION

Provided herein are compositions, kits, and methods for amplifying and/or detecting target nucleic acid from at least one pathogenic enteric parasite in a sample, wherein the at least one enteric parasite is selected from Cryptosporidium spp., Entamoeba histolytica, Giardia lamblia, and Cyclospora cayetanensis. Preferably, the samples are biological samples. The compositions, kits, and methods provide oligonucleotide sequences that target pathogenic enteric parasite gene sequences or their complementary sequences. Such oligonucleotides may be used as amplification oligonucleotides, which may include primers, promoter primers, blocked oligonucleotides, and promoter provider oligonucleotides, whose functions have been described previously (see, e.g., U.S. Pat. Nos. 4,683,195; 4,683,202; 4,800,159; 5,399,491; 5,554,516; 5,824,518; and 7,374,885; each incorporated by reference herein). Other oligonucleotides may be used as probes for detecting amplified sequences or for capture of an enteric parasite target nucleic acid.

The methods provide for the sensitive and specific detection of Cryptosporidium spp., Entamoeba histolytica, Giardia lamblia, and/or Cyclospora cayetanensis nucleic acids. In particular variations of methods that include Cryptosporidium spp. detection as described herein, the targeted Cryptosporidium spp. included one or more of C. parvum, C. hominis, C. meleagridis, C. baileyi, C. ubiquitum, and C. wrairi. In particular variations of methods that include Giardia lamblia detection as described herein, the methods target G. lamblia assemblage A and/or assemblage B.

The methods include performing nucleic acid amplification of a target region of one or more of Cryptosporidium spp., Entamoeba histolytica, Giardia lamblia, and Cyclospora cayetanensis, and detecting one or more amplified products by, for example, specifically hybridizing the amplified product(s) with one or more nucleic acid detection probes that provide a signal to indicate the presence of the at least one enteric parasite in the sample. The amplification step includes contacting the sample with (a) one or more Cryptosporidium-specific amplification oligomers specific for a target sequence in a Cryptosporidium spp. (e.g., C. parvum and/or C. hominis) target nucleic acid, (b) one or more Entamoeba-specific amplification oligomers specific for a target sequence in an Entamoeba histolytica target nucleic acid, (c) one or more Giardia-specific amplification oligomers specific for a target sequence in a Giardia lamblia (e.g., G. lamblia assemblage A and/or assemblage B) target nucleic acid, and/or (d) one or more Cyclospora-specific amplification oligomers specific for a target sequence in a Cyclospora cayetanensis target nucleic acid. Particularly suitable target nucleic acids include the 18S rRNA genes of Cryptosporidium spp., Entamoeba histolytica, Giardia lamblia, and/or Cyclospora cayetanensis (see. e.g., GenBank Accessions Nos. AF108865.1, DQ286403.1, X56991.1, M54878.1, AF199447.1, and KX618190.1, which show exemplary reference sequences for the 18S rRNA gene of C. parvum, C. hominis, E. histolytica, G. lamblia assemblage A. G. lamblia assemblage B, and C. cayetanensis, respectively). Nucleic acid amplification is performed to produce one or more amplification products corresponding to one or more of the Cryptosporidium spp., Entamoeba histolytica, Giardia lamblia, and/or Cyclospora cayetanensis target nucleic acids, if present in the sample, wherein the amplification reaction synthesizes additional copies of the target sequence or its complement by using at least one nucleic acid polymerase and the one or more amplification oligomers to produce the copies from a template strand (e.g., by extending the sequence from a primer using the template strand). One embodiment for detecting the amplification product uses a hybridizing step that includes contacting the amplified product with at least one detection probe oligomer specific for a sequence amplified by the selected amplification oligomers, e.g., a sequence contained in the target sequence flanked by a pair of selected amplification oligomers.

In some aspects, oligonucleotides are provided, e.g., in a kit or composition. Oligonucleotides generally comprise a target-hybridizing region, e.g., configured to hybridize specifically to a target nucleic acid of an enteric parasite selected from Cryptosporidium spp., Entamoeba histolytica, Giardia lamblia, and Cyclospora cayetanensis. While oligonucleotides of different lengths and base composition may be used for amplifying target nucleic acids, in some embodiments, oligonucleotides in this disclosure have target-hybridizing regions from about 10 to about 60 bases in length, from about 14 to about 50 bases in length, from about 14 to about 40 bases in length, from about 14 to about 35 bases in length, from about 15 to about 30 bases in length, or from about 16 to about 30 bases in length. In some embodiments, an oligonucleotide comprises a second region of sequence in addition to the target-hybridizing region, such as a promoter, which can be located 5′ of the target-hybridizing region. In some embodiments, an oligonucleotide does not comprise a second region of sequence.

In some embodiments, a set of oligonucleotides comprising a combination of two or more oligonucleotides are provided, e.g., in a kit or composition, such as an amplification oligomer (e.g., primer) pair or an amplification oligomer pair and a third oligonucleotide that is optionally labeled (e.g., for use as a probe), wherein the oligonucleotides are configured to hybridize to a target nucleic acid of at least one enteric parasite selected from Cryptosporidium spp., Entamoeba histolytica, Giardia lamblia, and Cyclospora cayetanensis. In some embodiments, the set of oligonucleotides comprises a plurality of amplification oligomer (e.g., primer) pairs or a plurality of amplification oligomer pairs and third oligonucleotides that are optionally labeled (e.g., for use as probes), wherein the oligonucleotides are configured to collectively hybridize to target nucleic acids of at least two enteric parasites selected from Cryptosporidium spp., Entamoeba histolytica, Giardia lamblia, and/or Cyclospora cayetanensis.

In some embodiments, one or more oligonucleotides comprise a non-Watson Crick (NWC) position. In some embodiments, a Cryptosporidium spp. amplification oligomer, a Cryptosporidium spp. amplification oligomer pair, and/or a Cryptosporidium spp. probe comprises a NWC position, such as a position that includes inosine. In some embodiments, an Entamoeba histolytica amplification oligomer, an Entamoeba histolytica amplification oligomer pair, and/or an Entamoeba histolytica probe comprises a NWC position, such as a position that includes inosine. In some embodiments, a Giardia lamblia amplification oligomer, a Giardia lamblia amplification oligomer pair, and/or a Giardia lamblia probe comprises a NWC position, such as a position that includes inosine. In some embodiments, a Cyclospora cayetanensis amplification oligomer, a Cyclospora cayetanensis amplification oligomer pair, and/or a Cyclospora cayetanensis probe comprises a NWC position, such as a position that includes inosine.

In some embodiments, one or more oligonucleotides comprise a position comprising 5-methylcytosine. In some embodiments, a Cryptosporidium spp. amplification oligomer, a Cryptosporidium spp. amplification oligomer pair, and/or a Cryptosporidium spp. probe comprises a position comprising 5-methylcytosine. In some embodiments, an Entamoeba histolytica amplification oligomer, an Entamoeba histolytica amplification oligomer pair, and/or an Entamoeba histolytica probe comprises a position comprising 5-methylcytosine. In some embodiments, a Giardia lamblia amplification oligomer, a Giardia lamblia amplification oligomer pair, and/or a Giardia lamblia probe comprises a position comprising 5-methylcytosine. In some embodiments, a Cyclospora cayetanensis amplification oligomer, a Cyclospora cayetanensis amplification oligomer pair, and/or a Cyclospora cayetanensis probe comprises a position comprising 5-methylcytosine.

In some embodiments, one or more oligonucleotides comprise a position comprising propyne dU. In some embodiments, a Cryptosporidium spp. amplification oligomer, a Cryptosporidium spp. amplification oligomer pair, and/or a Cryptosporidium spp. probe comprises a position comprising propyne dU. In some embodiments, an Entamoeba histolytica amplification oligomer, an Entamoeba histolytica amplification oligomer pair, and/or an Entamoeba histolytica probe comprises a position comprising propyne dU. In some embodiments, a Giardia lamblia amplification oligomer, a Giardia lamblia amplification oligomer pair, and/or a Giardia lamblia probe comprises a position comprising propyne dU. In some embodiments, a Cyclospora cayetanensis amplification oligomer, a Cyclospora cayetanensis amplification oligomer pair, and/or a Cyclospora cayetanensis probe comprises a position comprising propyne dU.

Exemplary oligomers targeting Cryptosporidium spp., Entamoeba histolytica, Giardia lamblia, or Cyclospora cayetanensis target nucleic acid in accordance with the present disclosure are shown in Table 26. Exemplary amplification oligomer pairs and optional third oligomers (e.g., detection probe) are set forth (by SEQ ID NO) in the following Table 1.

TABLE 1
Exemplary Oligonucleotide Sets

| | Oligonucleotide 1* | Oligonucleotide 2* | Oligonucleotide 3* |
|---|---|---|---|
| | (e.g., forward | (e.g., reverse | (e.g., probe, optionally |
| Target pathogen | primer) | primer) | labeled) |
| Cryptosporidium | 8 | 37 | 52 |
| spp. | 33 | 28 | 24 |
| | 4 | 37 | 48 |
| | 11 | 49 | 48 |
| Entamoeba | 46 | 19 | 17 |
| histolytica | 21 | 20 | 34 |
| | 36 | 43 | 7 |
| | 42 | 19 | 44 |
| Giardia lamblia | 25 | 2 | 39, 40, 41 |
| | 5, 6 | 29 | 32 |
| | 25 | 3 | 39, 40 |
| | 25 | 3 | 41 |
| Cyclospora | 26 | 14 | 16, 45 |
| cayetanensis | 23 | 18 | 12 |
| | 31 | 22 | 10 |
| | 35 | 13 | 47 |
| | 38 | 51 | 30 |
| | 1 | 9 | 50 |

*Oligonucleotides are referenced by SEQ ID NO and may include from 0 to 16 nucleotide analogs.

Exemplary oligomers containing one or more nucleotide analogues (“modified oligomers”) are set forth by SEQ ID NO in the following Table 2 below.

TABLE 2
Exemplary Modified Oligomers

| Modified Oligomer | Corresponds to |
|---|---|
| SEQ ID NO | SEQ ID NO* |
| 53 | 51 |
| 54 | 46 |
| 55 | 50 |
| 56 | 45 |
| 57 | 47 |
| 58 | 30 |
| 59 | 16 |
| 62 | 49 |
| 63 | 34 |
| 64 | 37 |
| 65 | 48 |
| 66 | 48 |
| 67 | 24 |
| 68 | 52 |
| 69 | 33 |
| 70 | 31 |
| 71 | 26 |
| 72 | 22 |
| 73 | 32 |
| 74 | 41 |
| 75 | 40 |
| 76 | 39 |
| 77 | 32 |
| 78 | 44 |
| 79 | 17 |
| 80 | 7 |
| 81 | 14 |
| 82 | 14 |
| 83 | 13 |
| 84 | 11 |
| 85 | 6 |
| 86 | 19 |
| 87 | 1 |
| 88 | 9 |
| 89 | 8 |
| 90 | 35 |
| 91 | 28 |
| 92 | 25 |
| 93 | 26 |

*“Corresponds to” means that the modified oligomer is an example of an oligomer containing one or more nucleotide analogues relative to this SEQ ID NO.

In some embodiments, an oligonucleotide is provided that comprises a label. Such an oligonucleotide can be used as a detection probe. In some embodiments, the labeled oligonucleotide has a sequence corresponding to a SEQ ID NO listed in the Oligonucleotide 3 column of Table 1. In some embodiments, the label is a non-nucleotide label. Suitable labels include compounds that emit a detectable light signal, e.g., fluorophores or luminescent (e.g., chemiluminescent) compounds that can be detected in a homogeneous mixture. More than one label, and more than one type of label, may be present on a particular probe, or detection may rely on using a mixture of probes, in which each probe is labeled with a compound that produces a detectable signal (see. e.g., U.S. Pat. Nos. 6,180,340 and 6,350,579, each incorporated by reference herein). Labels may be attached to a probe by various means including covalent linkages, chelation, and ionic interactions, but in some embodiments the label is covalently attached. For example, in some embodiments, a detection probe has an attached chemiluminescent label such as, e.g., an acridinium ester (AE) compound (see, e.g., U.S. Pat. Nos. 5,185,439; 5,639,604; 5,585,481; and 5,656,744). A label, such as a fluorescent or chemiluminescent label, can be attached to the probe by a non-nucleotide linker (see, e.g., U.S. Pat. Nos. 5,585,481; 5,656,744; and 5,639,604). In some embodiments, the label may include one or more of Quasar670, CalRed610, CalOrange560, fluorescein, ROX, FAM, and HEX.

In some embodiments, a detection probe (e.g., comprising a fluorescent label) further comprises a second label that interacts with the first label. For example, the second label can be a quencher. In some embodiments, the second label may include one or both of BHQ-1 and BHQ-2. Such probes can be used, e.g., in TaqMan™ assays, where hybridization of the probe to a target or amplicon followed by nucleolysis by a polymerase comprising 5′-3′ exonuclease activity results in liberation of the fluorescent label and thereby increased fluorescence, or fluorescence independent of the interaction with the second label.

In some applications, one or more detection probes exhibiting at least some degree of self-complementarity are used to facilitate detection of probe:target duplexes in a test sample without first requiring the removal of unhybridized probe prior to detection. Specific embodiments of such detection probes include, for example, probes that form conformations held by intramolecular hybridization, such as conformations generally referred to as hairpins.

Suitable hairpin probes include a “molecular torch” (see. e.g., U.S. Pat. Nos. 6,849,412; 6,835,542; 6,534,274; and 6,361,945) and a “molecular beacon” (see, e.g., U.S. Pat. Nos. 5,118,801 and 5,312,728). Molecular torches include distinct regions of self-complementarity (coined “the target-binding domain” and “the target-closing domain”) which are connected by a joining region (e.g., a —(CH₂CH₂O)₃— linker) and which hybridize to one another under predetermined hybridization assay conditions. When exposed to an appropriate target or denaturing conditions, the two complementary regions (which may be fully or partially complementary) of the molecular torch melt, leaving the target-binding domain available for hybridization to a target sequence when the predetermined hybridization assay conditions are restored. Molecular torches are designed so that the target-binding domain favors hybridization to the target sequence over the target-closing domain. The target-binding domain and the target-closing domain of a molecular torch include interacting labels (e.g., fluorescent/quencher) positioned so that a different signal is produced when the molecular torch is self-hybridized as opposed to when the molecular torch is hybridized to a target nucleic acid, thereby permitting detection of probe:target duplexes in a test sample in the presence of unhybridized probe having a viable label associated therewith.

Examples of interacting donor/acceptor label pairs that may be used in connection with the disclosure, making no attempt to distinguish FRET from non-FRET pairs, include fluorescein/tetramethylrhodamine, IAEDANS/fluorescein, EDANS/DABCYL, coumarin/DABCYL, fluorescein/fluorescein, BODIPY FL/BODIPY FL, fluorescein/DABCYL, lucifer yellow/DABCYL, BODIPY/DABCYL, eosine/DABCYL, erythrosine/DABCYL, tetramethylrhodamine/DABCYL, Texas Red/DABCYL, CY5/BHQ-1, CY5/BHQ-2, CY3/BHQ-1, CY3/BHQ-2 and fluorescein/QSY7 dye. Those having an ordinary level of skill in the art will understand that when donor and acceptor dyes are different, energy transfer can be detected by the appearance of sensitized fluorescence of the acceptor or by quenching of donor fluorescence. Non-fluorescent acceptors such as DABCYL and the QSY7 dyes advantageously eliminate the potential problem of background fluorescence resulting from direct (i.e., non-sensitized) acceptor excitation. Exemplary fluorophore moieties that can be used as one member of a donor-acceptor pair include fluorescein, ROX, the ATTO dyes, the DY dyes, and the CY dyes. Exemplary quencher moieties that can be used as another member of a donor-acceptor pair include DABCYL, BlackBerry Quencher, and the Black Hole Quencher moieties.

In some embodiments, a labeled oligonucleotide (e.g., probe) is non-extendable. For example, the labeled oligomer can be rendered non-extendable by 3′-phosphorylation, having a 3′-terminal 3′-deoxynucleotide (e.g., a terminal 2′,3′-dideoxynucleotide), having a 3′-terminal inverted nucleotide (e.g., in which the last nucleotide is inverted such that it is joined to the penultimate nucleotide by a 3′ to 3′ phosphodiester linkage or analog thereof, such as a phosphorothioate), or having an attached fluorophore, quencher, or other label that interferes with extension (possibly but not necessarily attached via the 3′ position of the terminal nucleotide). In some embodiments, the 3′-terminal nucleotide is not methylated.

Also provided by the disclosure is a reaction mixture for determining the presence or absence of a target nucleic acid of at least one pathogenic enteric parasite in accordance with the methods as described herein. A reaction mixture in accordance with the present disclosure comprises at least one or more of the following: an oligonucleotide as described herein for amplification of a target nucleic acid; and an oligonucleotide (e.g., probe) as described herein for determining the presence or absence of an amplification product of the target nucleic acid. For a reaction mixture that includes a detection probe together with an amplification oligonucleotide combination, the amplification oligonucleotides and detection probe oligonucleotides for a reaction mixture are linked by a common target region (i.e., the reaction mixture will include a probe that binds to a sequence amplifiable by an amplification oligonucleotides combination of the reaction mixture).

A reaction mixture may further include a number of optional components such as, for example, capture probes, e.g., poly-(k) capture probes as described in US 2013/0209992, which is incorporated herein by reference, and/or poly-(R) capture probes as described in US 2020/0165599, which is incorporated herein by reference. For an amplification reaction mixture, the reaction mixture will typically include other reagents suitable for performing in vitro amplification such as, e.g., buffers, salt solutions, appropriate nucleotide triphosphates (e.g., dATP, dCTP, dGTP, and dTTP; and/or ATP, CTP, GTP and UTP), and/or enzymes (e.g., a thermostable DNA polymerase, or reverse transcriptase and/or RNA polymerase), and will typically include test sample components, in which a target nucleic acid may or may not be present. Suitable reagents include, for example, formulations containing lithium lauryl sulfate (LLS), sodium lauryl sulfate (SLS), NaH₂PO₄, Na₂HPO₄, EDTA, EGTA, LiOH, NaCl, KCl, MgCl₂, NaOH, ethanol, methylparaben, propylparaben, trehalose, Tris Buffer, Triton X-100, paramagnetic particles, target capture oligonucleotides, HEPES, succinic acid, polymerases (e.g., DNA polymerases, reverse transcriptases), and/or RNasin.

In some embodiments, a reaction mixture comprises KCl. In some embodiments, the KCl concentration is about 50 mM. In some embodiments, the KCl concentration is greater than about 50 mM, e.g., about 60-150 mM, about 75-125 mM, about 80-120 mM, about 85-115 mM, or about 90-110 mM. In some embodiments, the KCl concentration is about 55-65 mM, about 65-75 mM, about 75-85 mM, about 85-95 mM, about 95-105 mM, about 105-115 mM, about 115-125 mM, about 125-135 mM, or about 135-145 mM. In some embodiments, a composition according to the disclosure comprises KCl, e.g., at any of the foregoing concentrations. In some embodiments, a method according to the disclosure comprises performing an amplification reaction in the presence of KCl, e.g., at any of the foregoing concentrations.

In some embodiments, a reaction mixture comprises a non-linear surfactant such as, for example, polysorbate 20. In certain variations, the non-linear surfactant (e.g., polysorbate 20) is present in the reaction mixture at a concentration of from about 0.001% to about 0.025% (v/v) or from about 0.0015% to about 0.015% (v/v). In certain embodiments, a reaction mixture comprises α-cyclodextrin. In certain variations, the α-cyclodextrin is present at a concentration from about 1.0 mg/mL to about 10 mg/mL, from about 3.0 mg/mL to about 9.0 mg/mL, or from about 2.0 mg/mL to about 7.0 mg/mL.

In some embodiments, a reaction mixture comprises a lyoprotectant. Exemplary lyoprotectants include glycerol; non-reducing sugars such as, e.g., sucrose, raffinose, or trehalose; and amino acids such as, e.g., glycine, arginine, or methionine. In certain variations wherein the lyoprotectant is trehalose, trehalose is present at a concentration of from about 0.1 M to about 0.2 M (e.g., about 0.15 M).

In some embodiments, a reaction mixture comprises a chelating agent. Suitable chelating agents include ethylenediaminetetraacetic acid (EDTA) and ethylene glycol-bis(β-aminoethyl ether)-N,N,N′,N′-tetraacetic acid (EGTA). In some embodiments comprising EDTA as the chelating agent, EDTA is present in the reaction mixture at a concentration of from about 0.025 mM to about 0.25 mM (e.g., at a concentration of about 0.08 mM).

Also provided by the subject disclosure are kits for practicing the methods as described herein. A kit in accordance with the present disclosure comprises at least one or more of the following: an oligonucleotide as described herein for amplification of a target nucleic acid; and an oligonucleotide (e.g., probe) as described herein for determining the presence or absence of an amplification product of the target nucleic acid. In some embodiments, any oligonucleotide combination described herein is present in the kit. The kits may further include a number of optional components such as, for example, capture probes, e.g., poly-(k) capture probes as described in US 2013/0209992 and/or poly-(R) capture probes as described in US 2020/0165599. Other reagents that may be present in the kits include reagents suitable for performing in vitro amplification such as, e.g., buffers, salt solutions, appropriate nucleotide triphosphates (e.g., dATP, dCTP, dGTP, dTTP; and/or ATP, CTP, GTP and UTP), and/or enzymes (e.g., a thermostable DNA polymerase, or a reverse transcriptase and/or RNA polymerase). Oligonucleotides as described herein may be packaged in a variety of different embodiments, and those skilled in the art will appreciate that the disclosure embraces many different kit configurations. For example, a kit may include amplification oligonucleotides for only one, two, three, or all of Cryptosporidium spp., Entamoeba histolytica, Giardia lamblia, and Cyclospora cayetanensis. In addition, for a kit that includes a detection probe together with an amplification oligomer combination, the amplification oligonucleotides and detection probe oligonucleotides for a reaction mixture are linked by a common target region (i.e., the reaction mixture will include a probe that binds to a sequence amplifiable by an amplification oligonucleotides combination of the reaction mixture). In certain embodiments, the kit further includes a set of instructions for practicing methods in accordance with the present disclosure, where the instructions may be associated with a package insert and/or the packaging of the kit or the components thereof.

In some embodiments of a kit as described herein, the oligonucleotides are contained in a formulation comprising at least one of a non-linear surfactant (e.g., polysorbate 20), α-cyclodextrin, a lyoprotectant (e.g., a non-reducing sugar such as sucrose, raffinose, or trehalose, or an amino acid such as glycine, arginine, or methionine), and a chelating agent (e.g., EDTA or EGTA). In some such embodiments, polysorbate 20 is present in the formulation at a concentration of from about 0.002% to about 0.05% (v/v) or about 0.003% to about 0.03% (v/v), α-cyclodextrin is present at a concentration from about 1.0 mg/mL to about 10 mg/mL or about 3.0 mg/mL to about 9 mg/mL, trehalose is present in the formulation at a concentration of from about 0.2 M to about 0.4 M (e.g., about 0.26 M or about 0.3 M), and/or EDTA is present in the formulation at a concentration of from about 0.05 mM to about 0.5 mM (e.g., about 0.16 mM or about 0.14 mM). In certain variations, the formulation is a lyophilized formulation. In some embodiments of a lyophilized formulation, the formulation is for reconstitution into an aqueous formulation containing polysorbate 20, trehalose, and/or EDTA at concentration(s) as specified above.

Also provided by the subject disclosure are methods (e.g., multiplex methods) for determining the presence or absence of at least one enteric parasite, including Cryptosporidium spp., Entamoeba histolytica, Giardia lamblia, and/or Cyclospora cayetanensis, in a sample by, for example, using one or more of the oligonucleotides disclosed herein. Any method disclosed herein is also to be understood as a disclosure of corresponding uses of materials involved in the method directed to the purpose of the method. Any of the oligonucleotides and any combinations (e.g., kits and compositions) comprising such an oligonucleotide are to be understood as also disclosed for use in detecting enteric parasite target nucleic acid and for use in the preparation of a composition for detecting enteric parasite target nucleic acid.

Broadly speaking, methods can comprise one or more of the following components: target capture, in which a target nucleic acid (e.g., from a sample, such as a clinical sample) is annealed to a capture oligomer: isolation, e.g., washing, to remove material not associated with a capture oligomer; amplification; and amplicon detection, e.g., amplicon quantification, which may be performed in real time with amplification. Certain embodiments involve each of the foregoing steps. Certain embodiments involve exponential amplification, optionally with a preceding linear amplification step. Certain embodiments involve exponential amplification and amplicon detection. Certain embodiments involve any two of the components listed above. Certain embodiments involve any two components listed adjacently above, e.g., washing and amplification, or amplification and detection.

Amplifying an enteric parasite target nucleic acid region utilizes an in vitro amplification reaction using at least two amplification oligomers that flank a target region to be amplified (e.g., one or more oriented in the sense direction and one or more oriented in the antisense direction for exponential amplification). Particularly suitable oligomer combinations for amplification of Cryptosporidium spp., Entamoeba histolytica, Giardia lamblia, and/or Cyclospora cayetanensis target regions are described herein. Exemplary amplification oligomers for amplifying enteric parasite target regions are listed in Table 26, infra (see also exemplary modified oligomers in Table 2, supra), and particular combinations of first and second amplification oligomers for each of Cryptosporidium spp., Entamoeba histolytica, Giardia lamblia, and/or Cyclospora cayetanensis are set forth herein (see. e.g., Embodiments section and Table 1, supra, and Examples 2-9, infra (including Tables 3, 13, 15, 17, 19, 21, and 24).

A detection method in accordance with the present disclosure can further include the step of obtaining the sample to be subjected to subsequent steps of the method. In certain embodiments, “obtaining” a sample to be used includes, for example, receiving the sample at a testing facility or other location where one or more steps of the method are performed, and/or retrieving the sample from a location (e.g., from storage or other depository) within a facility where one or more steps of the method are performed.

In certain embodiments, the method further includes purifying the enteric parasite target nucleic acid from other components in the sample, e.g., before an amplification, such as before a capture step. Such purification may include methods of separating and/or concentrating organisms contained in a sample from other sample components, or removing or degrading non-nucleic acid sample components, e.g., protein, carbohydrate, salt, lipid, etc. In some embodiments, purifying the target nucleic acid includes degrading nucleic acid in the sample, e.g., with DNase, and optionally removing or inactivating the DNase or removing degraded nucleic acid.

In particular embodiments comprising a target purification step, a target nucleic acid is captured specifically or non-specifically and separated from other sample components. Non-specific target capture methods may involve selective precipitation of nucleic acids from a substantially aqueous mixture, adherence of nucleic acids to a support that is washed to remove other sample components, or other means of physically separating nucleic acids from a mixture that contains pathogenic enteric parasite nucleic acid and other sample components.

Target capture typically occurs in a solution phase mixture that contains one or more capture probe oligomers that hybridize to the enteric parasite target nucleic acid under hybridizing conditions. For embodiments comprising a capture probe tail, the target:capture-probe complex is captured by adjusting the hybridization conditions so that the capture probe tail hybridizes to an immobilized probe. Certain embodiments use a particulate solid support, such as paramagnetic beads. Selective and non-specific target capture methods are also described, e.g., in U.S. Pat. No. 6,110,678 and International Patent Application Pub. No. WO 2008/016988, each incorporated by reference herein.

Isolation can follow capture, where, for example, the complex on the solid support is separated from other sample components. Isolation can be accomplished by any appropriate technique, e.g., washing a support associated with the enteric pathogen target nucleic acid one or more times (e.g., two or three times) to remove other sample components and/or unbound oligomer. In embodiments using a particulate solid support, such as paramagnetic beads, particles associated with the enteric pathogen target may be suspended in a washing solution and retrieved from the washing solution, in some embodiments by using magnetic attraction. To limit the number of handling steps, the enteric parasite target nucleic acid may be amplified by simply mixing the target region in the complex on the support with amplification oligomers and proceeding with amplification steps.

Exponentially amplifying a target sequence utilizes an in vitro amplification reaction using at least two amplification oligomers that flank a target region to be amplified. In some embodiments, at least one oligonucleotide as described above is provided. In some embodiments, a plurality of pairs of oligonucleotides is provided, wherein the plurality comprises oligonucleotides pairs configured to hybridize to at least one, two, three, or all of Cryptosporidium spp., Entamoeba histolytica, Giardia lamblia, and Cyclospora cayetanensis target nucleic acids. The amplification reaction can be cycled or isothermal. Suitable amplification methods include, for example, replicase-mediated amplification, polymerase chain reaction (PCR), ligase chain reaction (LCR), strand-displacement amplification (SDA), and transcription-mediated or transcription-associated amplification (TMA).

A detection step may be performed using any of a variety of known techniques to detect a signal specifically associated with the amplified target region, such as, e.g., by hybridizing the amplification product with a labeled detection probe and detecting a signal resulting from the labeled probe (including from label released from the probe following hybridization in some embodiments), performing electrophoresis on the sample and/or the amplification product, or determining the sequence of the amplification product. In some embodiments, the labeled probe comprises a second moiety, such as a quencher or other moiety that interacts with the first label, as discussed above. The detection step may also provide additional information on the amplified sequence, such as, e.g., all or a portion of its nucleic acid base sequence. Detection may be performed after the amplification reaction is completed or may be performed simultaneously with amplifying the target region, e.g., in real time. In one embodiment, the detection step allows homogeneous detection, e.g., detection of the hybridized probe without removal of unhybridized probe from the mixture (see. e.g., U.S. Pat. Nos. 5,639,604 and 5,283,174). In some embodiments, the nucleic acids are associated with a surface that results in a physical change, such as a detectable electrical change. Amplified nucleic acids may be detected by concentrating them in or on a matrix and detecting the nucleic acids or dyes associated with them (e.g., an intercalating agent such as ethidium bromide or cyber green) or detecting an increase in dye associated with nucleic acid in solution phase. Other methods of detection may use nucleic acid detection probes that are configured to specifically hybridize to a sequence in the amplified product and detecting the presence of the probe: product complex, or by using a complex of probes that may amplify the detectable signal associated with the amplified products (see, e.g., U.S. Pat. Nos. 5,424,413; 5,451,503; and 5,849,481; each incorporated by reference herein). Directly or indirectly labeled probes that specifically associate with the amplified product provide a detectable signal that indicates the presence of the target nucleic acid in the sample. In particular, the amplified product will contain a target sequence in or complementary to a target nucleic sequence of at least one pathogenic enteric parasite, and a probe will bind directly or indirectly to a sequence contained in the amplified product to indicate the presence or absence of the parasite in the tested sample.

In embodiments that detect the amplified product near or at the end of the amplification step, a linear detection probe may be used to provide a signal to indicate hybridization of the probe to the amplified product. One example of such detection uses a luminescently labeled probe that hybridizes to target nucleic acid. The luminescent label is then hydrolyzed from non-hybridized probe. Detection is performed by chemiluminescence using a luminometer. (See, e.g., International Patent Application Pub. No. WO 89/002476, incorporated by reference herein). In other embodiments that use real-time detection, the detection probe may be a hairpin probe such as, for example, a molecular beacon, molecular torch, or hybridization switch probe that is labeled with a reporter moiety that is detected when the probe binds to amplified product (e.g., a dual-labeled hairpin probe comprising both a fluorescent label and a quenching moiety). In other embodiments for real-time detection, the detection probe is a linear oligomer such as, e.g., an oligomer labeled with both a fluorophore and a quenching moiety (e.g., a TaqMan probe). Such probes may comprise target-hybridizing sequences and non-target-hybridizing sequences. Various forms of such probes have been described previously (see, e.g., U.S. Pat. Nos. 5,210,015; 5,487,972; 5,118,801; 5,312,728; 5,925,517; 6,150,097; 6,849,412; 6,835,542; 6,534,274; and 6,361,945; and US Patent Application Pub. Nos. 20060068417A1 and 20060194240A1: each incorporated by reference herein). Exemplary enteric-parasite-specific detection probe oligomers are listed in Tables 1 and 2, supra, and Table 26, infra, and are also set forth in the Embodiments section, supra, and Examples, infra (including, e.g., their use in combination with at least two enteric-parasite-specific amplification oligomers for detection of an enteric parasite target nucleic acid).

Assays for detection of an enteric parasite nucleic acid may optionally include a non-enteric-parasite internal control (IC) nucleic acid that is amplified and detected in the same assay reaction mixtures by using amplification and detection oligomers specific for the IC sequence. IC nucleic acid sequences can be, e.g., a DNA plasmid, an RNA template sequence (e.g., an in vitro transcript), or a synthetic nucleic acid that is spiked into a sample. Alternatively, the IC nucleic acid sequence may be a cellular component, which may be from exogenous cellular sources or endogenous cellular sources relative to the specimen. In these instances, an internal control nucleic acid is co-amplified with the enteric parasite nucleic acid in the amplification reaction mixtures. The internal control amplification product and the enteric parasite target region amplification product can be detected independently.

In certain embodiments, amplification and detection of a signal from an amplified IC sequence demonstrates that the assay reagents, conditions, and performance of assay steps were properly used in the assay if no signal is obtained for an intended target enteric parasite nucleic acid (e.g., samples that test negative for the enteric parasite). An IC may also be used as an internal calibrator for the assay when a quantitative result is desired, i.e., the signal obtained from the IC amplification and detection is used to set a parameter used in an algorithm for quantitating the amount of enteric parasite nucleic acid in a sample based on the signal obtained for an amplified enteric parasite target region. ICs are also useful for monitoring the integrity of one or more steps in an assay. The primers and probe for the IC target sequence are configured and synthesized by using any well-known method provided that the primers and probe function for amplification of the IC target sequence and detection of the amplified IC sequence using substantially the same assay conditions used to amplify and detect the enteric parasite target region(s). In certain embodiments that include a target capture-based purification step, it is preferred that a target capture probe specific for the IC target be included in the assay in the target capture step so that the IC is treated in the assay in a manner analogous to that for the intended enteric parasite analyte(s) in all of the assay steps.

Methods (e.g., multiplex methods) for determining the presence or absence of at least one enteric parasite as described herein may have a detection sensitivity of, for example, from 0.01 to 5 cells/mL, from 0.05 to 5 cells/mL, from 0.1 to 5 cells/mL, from 0.01 to 2.5 cells/mL, from 0.05 to 2.5 cells/mL, from 0.1 to 2.5 cells/mL, from 0.01 to 1 cells/mL, from 0.05 to 1 cells/mL, or from 0.1 to 1 cells/mL (e.g., for Cryptosporidium spp., G. lambia, or E. histolytica in CBS (Cary Blair Stool)). In other variations, methods (e.g., multiplex methods) for determining the presence or absence of at least one enteric parasite as described herein may have a detection sensitivity of, for example, from 0.001 to 0.5 cells/mL, from 0.005 to 0.5 cells/mL, from 0.01 to 0.5 cells/mL, from 0.001 to 0.25 cells/mL, from 0.005 to 0.25 cells/mL, from 0.01 to 0.25 cells/mL, from 0.001 to 0.1 cells/mL, from 0.005 to 0.1 cells/mL, or from 0.01 to 0.1 cells/mL (e.g., for Cryptosporidium spp., G. lamblia, or E. histolytica in Aptima® tube). In some embodiments comprising detection of Cyclospora cayetanensis, which is typically unculturable, sensitivity may be expressed as copies of C. cayetanensis in vitro transcript (IVT) per mL. For example, methods (e.g., multiplex methods) for determining the presence or absence of C. cayetanensis as described herein may have a detection sensitivity of from 1,000 to 100,000 cp/mL, from 5,000 to 100,000 cp/mL, from 10,000 to 100,000 cp/mL, from 15,000 to 100,000 cp/mL, from 1,000 to 50,000 cp/mL, from 5,000 to 50,000 cp/mL, from 10,000 to 50,000 cp/mL, from 15,000 to 50,000 cp/mL, from 1,000 to 30,000 cp/mL, from 5,000 to 30,000 cp/mL, from 10,000 to 30,000 cp/mL, or from 15,000 to 30,000 cp/mL (e.g., copies of IVT in CBS). In other variations, methods (e.g., multiplex methods) for determining the presence or absence of C. cayetanensis as described herein may have a detection sensitivity of from 50 to 5,000 cp/mL, from 250 to 5,000 cp/mL, from 500 to 5,000 cp/mL, from 750 to 5,000 cp/mL, from 50 to 2,500 cp/mL, from 250 to 2,500 cp/mL, from 500 to 2,500 cp/mL, from 750 to 2,500 cp/mL, from 50 to 1,500 cp/mL, from 250 to 1,500 cp/mL, from 500 to 1,500 cp/mL, or from 750 to 1,500 cp/mL (e.g., copies of IVT in Aptima® tube).

Also provided by the subject disclosure are methods for synthesizing one or more (e.g., one or more pairs) of the oligonucleotides disclosed herein, the oligonucleotides useful for determining the presence or absence of at least one enteric parasite selected from Cryptosporidium spp., Entamoeba histolytica, Giardia lamblia, and Cyclospora cayetanensis. The method may, for example, include the steps of (a) obtaining a solid support comprising at least one nucleobase residue, wherein the at least one nucleobase residue is bound (e.g., covalently bound) at a 3′ position to the solid support; (b) coupling a 5′ position of the nucleobase residue furthest from the solid support to a 3′ position of another nucleobase residue: (c) repeating step (b) at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, or at least 28 additional times, thereby generating at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 contiguous nucleobase residues coupled to the solid support; and (d) cleaving the at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 contiguous nucleobase residues generated in step (c), thereby obtaining the oligonucleotide or oligonucleotides. In some embodiments, the oligonucleotide has a length of from 16 to 32, from 16 to 30, or from 18 to 30 contiguous nucleobase residues.

A method for synthesizing one or more of the oligonucleotides disclosed herein may be a solid phase method. For example, phosphoramidite solid-phase chemistry for joining nucleotides by phosphodiester linkages is disclosed in Caruthers et al., “Chemical Synthesis of Deoxynucleotides by the Phosphoramidite Method,” Methods Enzymol. 154:287 (1987). As another example, automated solid-phase chemical synthesis using cyanoethyl phosphoramidite precursors has been described in Barone et al., “In Situ Activation of bis-dialkylaminephosphines—a New Method for Synthesizing Deoxyoligonucleotides on Polymer Supports,” Nucleic Acids Res. 12(10):4051 (1984). As another example, U.S. Pat. No. 5,449,769, titled “Method and Reagent for Sulfurization of Organophosphorous Compounds,” discloses a procedure for synthesizing oligonucleotides containing phosphorothioate linkages.

In addition, U.S. Pat. No. 5,811,538, titled “Process for the Purification of Oligomers,” discloses the synthesis of oligonucleotides having different linkages, including methylphosphonate linkages. Moreover, methods for the organic synthesis of oligonucleotides are described in, for example, Sambrook et al., Molecular Cloning, A Laboratory Manual, 2nd ed. (Cold Spring Harbor Laboratory Press, Cold Spring Harbor, NY, 1989) at Ch. 10.

Following synthesis and purification of a particular oligonucleotide, several different procedures may be utilized to purify and control the quality of the oligonucleotide. Suitable procedures include electrophoresis (e.g., polyacrylamide gel electrophoresis) or chromatography (e.g., high pressure liquid chromatography).

The compositions, kits, formulations, reaction mixtures, and methods are further illustrated by the following non-limiting examples.

### EXAMPLES

### Example 1: Real-Time PCR Amplification and Detection of GI-Bacterial Panel Targets Using Different Combinations of Primers and Probes

Several primer and probe combinations for real-time PCR amplification and detection of Cryptosporidium spp., Entamoeba histolytica, Giardia lamblia, and Cyclospora cayetanensis targets were tested.

Amplification and detection reactions were performed using a Panther Fusion instrument (Hologic, Inc. San Diego, CA). Typically, 20 μL of an amplification reagent was combined in a reaction well of a multi-well plate with 5 μL of a target nucleic acid. The multi-well plate was placed in the Panther Fusion instrument and subjected to thermal cycling. Real-time amplification and detection reactions were performed by thermal cycling, generally for 45 cycles (denaturation at 95° C. for 8 seconds and annealing and extension at 60° C. for 25 seconds), taking fluorescent emission readings every 30 seconds. Fluorescence curve profiles for the target nucleic acids were evaluated for Ct and RFU signals. The assay targeted DNA and did not include a reverse transcriptase (RT) extension step.

### Example 2: Evaluation of Oligonucleotides for Analytical Sensitivity

The primers and probes shown in Table 3 were evaluated for analytical sensitivity.

TABLE 3

| | Oligo | SEQ ID | |
|---|---|---|---|
| Target | Type | NO | Modifications |
| Cryptosporidium spp. | Primer | 8 | 5 mC at residues 2, 3, 6, and 15 |
| | | | pdU at residues 7, 11, and 17 |
| | Primer | 37 | 5 mC at residues 2, 5, 7, 11, and 12 |
| | Probe | 52 | 5 mC at residues 5, 9, 12, and 22 |
| | | | pdU at residues 2, 4, 8, 10, 14, 23, and 25 |
| | | | FAM/BHQ1 |
| | | | Reverse polarity C |
| E. histolytica | Primer | 46 | 5 mC at residues 3 and 17 |
| | Primer | 19 | 5 mC at residues 1, 9, 11, and 20 |
| | | | pdU at residues 7, 13, 17, and 22 |
| | Probe | 17 | 5 mC at residues 4, 21, 22, and 24 |
| | | | pdU at residues 2, 3, 5, 9, 13, 14, and 19 |
| | | | CalOrange560/BHQ1 |
| G. lamblia | Primer | 25 | 5 mC at residues 1, 4, 6, and 8 |
| | Primer | 2 | |
| | Probe | 41 | 5 mC at residues 7, 11, 15, and 20 |
| | | | CalRed610/BHQ2 |
| | Probe | 39 | 5 mC at residues 7, 11, 15, and 20 |
| | | | CalRed610/BHQ2 |
| | Probe | 40 | 5 mC at residues 7, 11, 15, and 20 |
| | | | CalRed610/BHQ2 |
| C. cayetanensis | Primer | 27 | 5 mC at residues 1, 3, and 10-12 |
| | | | pdU at residues 7, 8, and 15 |
| | Primer | 15 | 5 mC at residues 8 and 10 |
| | | | pdU at residues 6, 11, and 20 |
| | Probe | 16 | 5 mC at residues 4-6, 20, and 22 |
| | | | pdU at residues 2, 7, 12, 15, 18, and 21 |
| | | | Quasar670/BHQ2 |

Limit of detection (LoD) of the assay was first tested with different concentrations of the target organisms in cells/mL, and 95% detection was obtained initially at the values in Table 4 below. This initial assay was run using the oligos in multiplex.

TABLE 4

| | LOD |
|---|---|
| | (cells/mL in Aptima ® |
| Target | tube) |
| C. hominis | 0.031 |
| C. parvum | 0.050 |
| E. histolytica | 0.007 |
| G. lamblia assemblage A | 0.003 |
| G. lamblia assemblage B | 0.020 |
| C. cayetanensis | 1032 copies/mL* |

*LoD measured in copies of plasmid per mL because Cyclospora is not culturable

An experiment was then performed to confirm these initial LoD values. Table 5 below shows the results of testing at LoD (1×) and at 3×LoD. Targets were tested in singleplex for each of the targets indicated in Table 4 and in multiplex (“Multi”) for C. parvum, E. histolytica, G. lamblia assemblage A, and C. cayetanensis (pooling C. parvum, E. histolytica, and G. lamblia assemblage A cells with C. cayetanensis in vitro transcript (IVT)).

TABLE 5
LoD Confirmation Results

| | | | | | | | | |
|---|---|---|---|---|---|---|---|---|
| | Conc. | % | Average | | Average | | Slope at | Background |
| Target | (x LoD) | Positive | Ct | SD Ct | RFU | SD RFU | threshold | RFU |

FAM

| | | | | | | | | |
|---|---|---|---|---|---|---|---|---|
| C. hominis | 1x | 100% | 33.78 | 1.34 | 3,346 | 1,145 | 254 | 4,547 |
| | 3x | 100% | 30.73 | 0.21 | 10,372 | 2,838 | 483 | 4,236 |
| C. parvum | 1x | 100% | 32.21 | 0.79 | 3,668 | 1,059 | 303 | 3,594 |
| | 3x | 100% | 29.40 | 0.18 | 14,027 | 3,741 | 607 | 3,484 |
| Multi | 1x | 100% | 32.71 | 0.70 | 4,131 | 1,440 | 294 | 4,378 |
| (C. parvum) | 3x | 100% | 29.92 | 0.20 | 14,139 | 3,117 | 521 | 4,217 |

HEX

| | | | | | | | | |
|---|---|---|---|---|---|---|---|---|
| E. histolytica | 1x | 95% | 38.37 | 1.88 | 878 | 355 | 88 | 522 |
| | 3x | 100% | 35.12 | 0.60 | 1,885 | 418 | 151 | 496 |
| Multi | 1x | 95% | 38.44 | 1.65 | 832 | 233 | 81 | 522 |
| | 3x | 100% | 35.24 | 0.85 | 1,869 | 495 | 152 | 488 |

ROX

| | | | | | | | | |
|---|---|---|---|---|---|---|---|---|
| Giardia A | 1x | 55% | 38.59 | 1.30 | 429 | 289 | 68 | 294 |
| | 3x | 100% | 36.21 | 0.79 | 952 | 182 | 113 | 285 |
| Giardia B | 1x | 95% | 37.84 | 2.07 | 752 | 250 | 84 | 282 |
| | 3x | 100% | 34.18 | 0.36 | 1,614 | 218 | 170 | 288 |
| Multi | 1x | 100% | 37.83 | 1.77 | 741 | 185 | 80 | 308 |
| (Giardia B) | 3x | 100% | 34.38 | 0.59 | 1,623 | 430 | 163 | 265 |

Quasar 670

| | | | | | | | | |
|---|---|---|---|---|---|---|---|---|
| Cyclospora | 1x | 100% | 35.71 | 0.77 | 3,000 | 818 | 279 | 86 |
| | 3x | 100% | 33.40 | 0.20 | 5,912 | 887 | 477 | 874 |
| Multi | 1x | 100% | 36.58 | 1.39 | 2,452 | 718 | 225 | 886 |
| | 3x | 100% | 33.18 | 0.26 | 5,746 | 848 | 444 | 837 |

The final LoD for each assay target is shown in Table 6 below.

TABLE 6
Confirmed LoD

| | LoD |
|---|---|
| | (cells/mL in Aptima ® |
| Target | tube) |
| C. hominis | 0.031 |
| C. parvum | 0.050 |
| E. histolytica | 0.007 |
| G. lamblia assemblage A | 0.009 |
| G. lamblia assemblage B | 0.020 |
| C. cayetanensis | 1032 copies/mL |

### Example 3: Evaluation of Oligonucleotides for Specificity (Cross-Reactivity)

The primers and probes shown in Table 3 (see Example 2, supra) were evaluated for specificity (cross-reactivity) against all the organisms shown in Table 7 below.

TABLE 7
Organisms Evaluated for Cross-reactivity

| | | | Test | |
|---|---|---|---|---|
| Panel | Microorganism | Type | Concentration | Units |
| 1 | Abiotrophia defectiva | cells | 1.00E+06 | CFU/ml |
| | Acinetobacter baumannii | cells | 1.00E+06 | CFU/ml |
| | Acinetobacter lwoffii | cells | 1.00E+06 | CFU/ml |
| | Aeromonas hydrophila | cells | 1.00E+06 | CFU/ml |
| | Alcaligenes faecalis | cells | 1.00E+06 | CFU/ml |
| | Anaerococcus tetradius | cells | 1.00E+06 | CFU/ml |
| 2 | Anaerococcus vaginalis | cells | 1.00E+06 | CFU/ml |
| | Arcobacter butzleri | cells | 1.00E+06 | CFU/ml |
| | Bacillus cereus | cells | 1.00E+06 | CFU/ml |
| | Bacteroides fragilis | cells | 1.00E+06 | CFU/ml |
| | Bacteroides thetaiotaomicron | cells | 1.00E+06 | CFU/ml |
| | Bacteroides vulgatus | cells | 1.00E+06 | CFU/ml |
| 3 | Bifidobacterium adolescentis | cells | 1.00E+06 | CFU/ml |
| | Bifidobacterium longum | cells | 1.00E+06 | rRNA Copies/ml |
| | Campylobacter fetus | cells | 1.00E+06 | CFU/ml |
| | Campylobacter hyointestinalis | cells | 1.00E+06 | CFU/ml |
| | Campylobacter rectus | cells | 1.00E+06 | CFU/ml |
| | Campylobacter sputorum | cells | 1.00E+06 | CFU/ml |
| 4 | Candida albicans | cells | 1.00E+06 | CFU/ml |
| | Citrobacter freundii | cells | 1.00E+06 | CFU/ml |
| | Citrobacter koseri | cells | 1.00E+06 | CFU/ml |
| | Clostridium difficile | cells | 1.00E+06 | CFU/ml |
| | Clostridium perfringens | cells | 1.00E+06 | CFU/ml |
| | Clostridium ramosum | cells | 1.00E+06 | CFU/ml |
| 5 | Clostridium sordellii | cells | 1.00E+06 | CFU/ml |
| | Clostridium tertium | cells | 1.00E+06 | CFU/ml |
| | Collinsella aerofaciens | cells | 1.00E+06 | CFU/ml |
| | Corynebacterium genitalium | cells | 1.00E+06 | CFU/ml |
| | Cronobacter sakazakii | cells | 1.00E+06 | CFU/ml |
| | Edwardsiella tarda | cells | 1.00E+06 | CFU/ml |
| 6 | Eggerthella lenta | cells | 1.00E+06 | rRNA Copies/ml |
| | Entercoccus faecalis | cells | 1.00E+06 | CFU/ml |
| | Enterobacter aerogenes | cells | 1.00E+06 | CFU/ml |
| | Enterobacter cloacae | cells | 1.00E+06 | CFU/ml |
| | Escherichia coli | cells | 1.00E+06 | CFU/ml |
| 7 | Escherichia coli* | cells | 1.00E+06 | CFU/ml |
| | Escherichia fergusonii | cells | 1.00E+06 | CFU/ml |
| | Escherichia hermanii | cells | 1.00E+06 | CFU/ml |
| | Escherichia vulneris | cells | 1.00E+06 | CFU/ml |
| | Gardnerella vaginalis | cells | 1.00E+06 | CFU/ml |
| | Helicobacter pylori | cells | 1.00E+06 | CFU/ml |
| 8 | Klebsiella oxytoca | cells | 1.00E+06 | CFU/ml |
| | Klebsiella ozaenae | cells | 1.00E+06 | CFU/ml |
| | Klebsiella pneumoniae | cells | 1.00E+06 | CFU/ml |
| | Lactobacillus acidophilus | cells | 1.00E+06 | CFU/ml |
| | Lactobacillus crispatus | cells | 1.00E+06 | CFU/ml |
| | Lactococcus lactis | cells | 1.00E+06 | CFU/ml |
| 9 | Leptotrichia buccalis | cells | 1.00E+06 | CFU/ml |
| | Listeria grayi | cells | 1.00E+06 | CFU/ml |
| | Listeria monocytogenes | cells | 1.00E+06 | CFU/ml |
| | Megasphaera elsdenii | cells | 1.00E+06 | CFU/ml |
| | Morganella morganii | cells | 1.00E+06 | CFU/ml |
| | Peptostreptococcus anaerobius | cells | 1.00E+06 | CFU/ml |
| 10 | Peptostreptococcus micros | cells | 1.00E+06 | rRNA Copies/ml |
| | Photobacterium damselae | cells | 1.00E+06 | CFU/ml |
| | Plesiomonas shigelloides | cells | 1.00E+06 | CFU/ml |
| | Prevotella bivia | cells | 1.00E+06 | CFU/ml |
| | Prevotella melaninogenica | cells | 1.00E+06 | CFU/ml |
| | Proteus mirabilis | cells | 1.00E+06 | CFU/ml |
| 11 | Proteus penneri | cells | 1.00E+06 | CFU/ml |
| | Proteus vulgaris | cells | 1.00E+06 | CFU/ml |
| | Providencia alcalifaciens | cells | 1.00E+06 | CFU/ml |
| | Providencia rettgeri | cells | 1.00E+06 | CFU/ml |
| | Providencia stuartii | cells | 1.00E+06 | CFU/ml |
| | Pseudomonas aeruginosa | cells | 1.00E+06 | CFU/ml |
| 12 | Pseudomonas fluorescens | cells | 1.00E+06 | CFU/ml |
| | Serratia liquefaciens | cells | 1.00E+06 | CFU/ml |
| | Serratia marcescens | cells | 1.00E+06 | CFU/ml |
| | Staphylococcus aureus | cells | 1.00E+06 | CFU/ml |
| | Staphylococcus epidermidis | cells | 1.00E+06 | CFU/ml |
| | Stenotrophomonas maltophilia | cells | 1.00E+06 | CFU/ml |
| 13 | Streptococcus anginosus | cells | 1.00E+06 | CFU/ml |
| | Streptococcus dysgalactiae | cells | 1.00E+06 | CFU/ml |
| | Yersinia bercovieri | cells | 1.00E+06 | CFU/ml |
| | Yersinia pseudotuberculosis | cells | 1.00E+06 | CFU/ml |
| | Yersinia rohdei | cells | 1.00E+06 | CFU/ml |
| 14 | Escherichia coli O124 | cells | 1.00E+06 | CFU/ml |
| 15 | Escherichia coli O29 | cells | 1.00E+06 | CFU/ml |
| 16 | Campylobacter lari | cells | 1.00E+06 | CFU/ml |
| 17 | Campylobacter upsaliensis | cells | 1.00E+06 | CFU/ml |
| 18 | Conidiobolus lachnodes | cells | 1.00E+03 | cells/mL |
| | Conidiobolus lobatus | cells | 1.00E+03 | cells/mL |
| | Blastocystis hominis | cells | 1.00E+04 | cells/mL |
| | Toxoplasma gondii | cells | 1.00E+04 | cells/mL |
| 19 | AstroV-ORF1B-in7 | IVT | 1.00E+05 | copies/mL |
| | GB NoV GI | IVT | 1.00E+05 | copies/mL |
| | GB SV (GI) ORF1 | IVT | 1.00E+05 | copies/mL |
| | GB SV GII ORF1 | IVT | 1.00E+05 | copies/mL |
| | GB SV GIV ORF1 IVT | IVT | 1.00E+05 | copies/mL |
| 20 | GB SV GV ORF1 IVT | IVI | 1.00E+05 | copies/mL |
| | NoroV GII ORF1-2 set1 1A | IVT | 1.00E+05 | copies/mL |
| | Adenovirus Type 40 Dugan | viral particles | 1.00E+02 | TCID50/mL |
| | Rotavirus WA | viral particles | 1.00E+01 | TCID50/mL |
| 21 | Salmonella enterica | cells | 1.00E+06 | CFU/mL |
| | Campylobacter coli | cells | 1.00E+06 | CFU/mL |
| | Shigella flexneri | cells | 1.00E+06 | CFU/mL |
| | Escherichia coli | cells | 1.00E+06 | CFU/mL |
| 22 | Plesimonas shigelloides | cells | 1.00E+06 | CFU/mL |
| | Yersinia enterolitica | cells | 1.00E+06 | CFU/mL |
| | Escherichia coli O157:H7 | cells | 1.00E+06 | CFU/mL |
| | Vibrio cholerae | cells | 1.00E+06 | CFU/mL |
| | Vibrio parahaemolyticus | cells | 1.00E+06 | CFU/mL |
| | Vibrio vulnificus | cells | 1.00E+06 | CFU/mL |
| 23 | Blastocystis hominis CLINICAL | clinical sample | Unknown | n/a |
| 24 | Endolimax nana | clinical sample | Unknown | n/a |
| 25 | Entamoeba coli | clinical sample | Unknown | n/a |
| 26 | Enterobius vermicularis | clinical sample | Unknown | n/a |
| 27 | Presence of anguillula larva | clinical sample | Unknown | n/a |
| 28 | Pseudolimax butschlii | clinical sample | Unknown | n/a |
| 29 | Entamoeba dispar | cells | 1.00E+04 | cells/mL. |
| | Entamoeba moshkovskii | cells | 1.00E+04 | cells/mL |
| | Entamoeba gingivalis | cells | 1.00E+04 | cells/mL |
| | Encephalitozoon intestinalis | cells | 1.00E+05 | cells/mL |
| | Encephalitozoon cuniculi | cells | 1.00E+05 | cells/mL |
| | Encephalitozoon hellem | cells | 1.00E+05 | cells/mL |
| 30 | Enterobacter cloacae | cells | 1.00E+06 | CFU/mL |
| 31 | Shigella sonnei | cells | 1.00E+06 | CFU/mL |
| 32 | Faecalibacterium prausnitzii | cells | 1.00E+06 | rRNA Copies/ml |
| | Bacillus subtilis | cells | 1.00E+06 | CFU/ml |
| | Eggerthella lenta | cells | 1.00E+06 | CFU/ml |
| | Corynebacterium genitalium | cells | 1.00E+06 | CFU/ml |
| | Pentatrichomonas hominis | cells | 1.00E+06 | CFU/ml |
| | Chlamydia trachomatis | cells | 4.00E+04 | IFU/mL |
| 33 | Hafnia alvei | cells | 1.00E+06 | CFU/ml |
| | Serratia marcescens | cells | 1.00E+06 | CFU/ml |
| | Enterococcus faecalis | cells | 1.00E+06 | CFU/ml |
| | Enterococcus faecium | cells | 1.00E+06 | CFU/ml |
| | Proteus vulgaris | cells | 1.00E+06 | CFU/ml |
| | Gemella morbillorum | cells | 1.00E+06 | CFU/ml |
| 34 | Mycoplasma fermentans | cells | 1.00E+06 | CFU/ml |
| | Atopobium vaginae | cells | 1.00E+06 | rRNA Copies/ml |
| | Veillonella parvula | cells | 1.00E+06 | CFU/ml |
| | Cytomegalovirus | viral particles | 3.00E+03 | TCID50/mL |
| | Trabulsiella guamensis | cells | 1.00E+06 | CFU/ml |
| | Arcobacter cryaerophilus | cells | 1.00E+06 | CFU/ml |
| | Salmonella enterica | cells | 1.00E+06 | CFU/ml |
| 35 | Necator americanus | plasmid | 1.00E+06 | copies/mL |
| | Ancylostoma duodenale | plasmid | 1.00E+06 | copies/mL |
| | Entamoeba nuttalli | plasmid | 1.00E+06 | copies/mL |
| | Entamoeba bangladeshi | plasmid | 1.00E+06 | copies/mL |
| | Entamoeba hartmanni | plasmid | 1.00E+06 | copies/mL |
| 36A | Cryptosporidium andersoni | plasmid | 1.00E+06 | copies/mL |
| 36B | Cryptosporidium felis | plasmid | 1.00E+06 | copies/mL |
| 37 | Cryptosporidium canis | plasmid | 1.00E+06 | copies/mL |
| | Cryptosporidium meleagridis | plasmid | 1.00E+06 | copies/mL |
| | Cryptosporidium baileyi | plasmid | 1.00E+06 | copies/mL |
| | Cryptosporidium ubiquitum | plasmid | 1.00E+06 | copies/mL. |
| | Cryptosporidium wrairi | plasmid | 1.00E+06 | copies/mL |
| | Cryptosporidium muris | plasmid | 1.00E+06 | copies/mL |
| 38 | Entamoeba ranarum | plasmid | 1.00E+06 | copies/mL |
| | Entamoeba invadens | plasmid | 1.00E+06 | copies/mL |
| | Ancylostoma ceylanicum | plasmid | 1.00E+06 | copies/mL |
| | Entamoeba chattoni | plasmid | 1.00E+06 | copies/mL |
| | Entamoeba polecki | plasmid | 1.00E+06 | copies/mL. |
| | Entamoeba coli | plasmid | 1.00E+06 | copies/mL |
| 39 | Actinomyces naeslundii | cells | 1.00E+06 | CFU/mL |
| | Akkermansia muciniphila | cells | 1.00E+06 | CFU/mL |
| | Aspergillus fumigatus | cells | 3.00E+05 | CFU/mL |
| | Capnocytophaga gingivalis | cells | 1.00E+06 | CFU/mL |
| | Cedecea davisae | cells | 1.00E+06 | CFU/mL |
| | Chryseobacterium gleum | cells | 1.00E+06 | CFU/mL |
| 40 | Coronavirus 229E | viral particles | 1.00E+05 | TCID50/mL |
| | Corynebacterium glutamicum | cells | 1.00E+06 | CFU/mL |
| | Coxsackievirus | viral particles | 3.00E+05 | TCID50/mL |
| | Desulfovibrio piger | cells | 1.00E+06 | CFU/mL |
| | Echovirus | viral particles | 1.00E+06 | TCID50/mL |
| | Enterovirus | viral particles | 1.00E+06 | TCID50/mL |
| 41 | Eubacterium rectale | cells | 1.00E+06 | copies rRNA/mL |
| | Fusobacterium varium | cells | 1.00E+06 | CFU/mL |
| | Helicobacter fennelliae | cells | 1.00E+06 | copies rRNA/mL |
| | Leminorella grimontii | cells | 1.00E+06 | CFU/mL |
| | Peptoniphilus asaccharolyticus | cells | 1.00E+06 | CFU/mL |
| 42 | Porphyromonas asaccharolytica | cells | 1.00E+06 | CFU/mL |
| | Porphyromonas levii | cells | 1.00E+06 | CFU/mL |
| | Pseudomonas putida | cells | 1.00E+06 | CFU/mL |
| | Ruminococcus bromii | cells | 1.00E+06 | CFU/mL |
| | Saccharomyces cerevisiae | cells | 3.00E+05 | CFU/mL |
| | Salmonella bongori | cells | 1.00E+06 | CFU/mL |
| 43 | Selenomonas ruminantium | cells | 1.00E+06 | CFU/mL |
| | Shigella dysenteriae | cells | 1.00E+06 | cells/ml |
| | Veillonella atypica | cells | 1.00E+06 | CFU/mL |
| 44 | HCV clinical specimen | clinical sample | Unknown | n/a |

Results of the experiment are shown in Table 8 below. The panels shown in bold underline were cross-reactive. When the panels were separated into single organisms, it was identified that the assay cross-reacted with E. nutalli (a species that can rarely infect humans) and some Cryptosporidium species (C. meleagridis, C. baileyi, C. ubiquitum, and C. wrairi) (see Table 9 below).

TABLE 8
Cross-reactivity Results

| | N positive/N tested | | | | | Average CT | | | | |
|---|---|---|---|---|---|---|---|---|---|---|
| | FAM | HEX | ROX | Q670 | Q705 | FAM | HEX | ROX | Q670 | Q705 |
| Panel | (Crypto) | (E hist) | (Giardia) | (Cyclo) | (IC) | (Crypto) | (E hist) | (Giardia) | (Cyclo) | (IC) |
| 1 | 0/3 | 0/3 | 0/3 | 0/3 | 3/3 | N/A | N/A | N/A | N/A | 30.68 |
| 2 | 0/3 | 0/3 | 0/3 | 0/3 | 3/3 | N/A | N/A | N/A | N/A | 30.76 |
| 3 | 0/3 | 0/3 | 0/3 | 0/3 | 3/3 | N/A | N/A | N/A | N/A | 30.64 |
| 4 | 0/3 | 0/3 | 0/3 | 0/3 | 3/3 | N/A | N/A | N/A | N/A | 30.72 |
| 5 | 0/3 | 0/3 | 0/3 | 0/3 | 3/3 | N/A | N/A | N/A | N/A | 30.60 |
| 6 | 0/3 | 0/3 | 0/3 | 0/3 | 3/3 | N/A | N/A | N/A | N/A | 30.72 |
| 7 | 0/3 | 0/3 | 0/3 | 0/3 | 3/3 | N/A | N/A | N/A | N/A | 30.68 |
| 8 | 0/3 | 0/3 | 0/3 | 0/3 | 3/3 | N/A | N/A | N/A | N/A | 30.55 |
| 9 | 0/3 | 0/3 | 0/3 | 0/3 | 3/3 | N/A | N/A | N/A | N/A | 30.53 |
| 10 | 0/3 | 0/3 | 0/3 | 0/3 | 3/3 | N/A | N/A | N/A | N/A | 30.75 |
| 11 | 0/3 | 0/3 | 0/3 | 0/3 | 3/3 | N/A | N/A | N/A | N/A | 30.47 |
| 12 | 0/3 | 0/3 | 0/3 | 0/3 | 3/3 | N/A | N/A | N/A | N/A | 30.64 |
| 13 | 0/3 | 0/3 | 0/3 | 0/3 | 3/3 | N/A | N/A | N/A | N/A | 30.54 |
| 14 | 0/3 | 0/3 | 0/3 | 0/3 | 3/3 | N/A | N/A | N/A | N/A | 30.63 |
| 15 | 0/3 | 0/3 | 0/3 | 0/3 | 3/3 | N/A | N/A | N/A | N/A | 30.70 |
| 16 | 0/3 | 0/3 | 0/3 | 0/3 | 3/3 | N/A | N/A | N/A | N/A | 30.64 |
| 17 | 0/3 | 0/3 | 0/3 | 0/3 | 3/3 | N/A | N/A | N/A | N/A | 30.62 |
| 18 | 0/3 | 0/3 | 0/3 | 0/3 | 3/3 | N/A | N/A | N/A | N/A | 30.48 |
| 19 | 0/3 | 0/3 | 0/3 | 0/3 | 3/3 | N/A | N/A | N/A | N/A | 30.56 |
| 20 | 0/3 | 0/3 | 0/3 | 0/3 | 3/3 | N/A | N/A | N/A | N/A | 30.56 |
| 21 | 0/3 | 0/3 | 0/3 | 0/3 | 3/3 | N/A | N/A | N/A | N/A | 30.66 |
| 22 | 0/3 | 0/3 | 0/3 | 0/3 | 3/3 | N/A | N/A | N/A | N/A | 30.68 |
| 23 | 0/3 | 0/3 | 0/3 | 0/3 | 3/3 | N/A | N/A | N/A | N/A | 30.31 |
| 24 | 0/3 | 0/3 | 0/3 | 0/3 | 3/3 | N/A | N/A | N/A | N/A | 29.74 |
| 25 | 0/3 | 0/3 | 0/3 | 0/3 | 3/3 | N/A | N/A | N/A | N/A | 31.06 |
| 26 | 0/3 | 0/3 | 0/3 | 0/3 | 3/3 | N/A | N/A | N/A | N/A | 29.48 |
| 27 | 0/3 | 0/3 | 0/3 | 0/3 | 3/3 | N/A | N/A | N/A | N/A | 29.91 |
| 28 | 0/3 | 0/3 | 0/3 | 0/3 | 3/3 | N/A | N/A | N/A | N/A | 29.63 |
| 29 | 0/3 | 0/3 | 0/3 | 0/3 | 3/3 | N/A | N/A | N/A | N/A | 30.30 |
| 30 | 0/3 | 0/3 | 0/3 | 0/3 | 3/3 | N/A | N/A | N/A | N/A | 30.62 |
| 31 | 0/3 | 0/3 | 0/3 | 0/3 | 3/3 | N/A | N/A | N/A | N/A | 30.52 |
| 32 | 0/5 | 0/5 | 0/5 | 0/5 | 5/5 | N/A | N/A | N/A | N/A | 29.73 |
| 33 | 0/3 | 0/3 | 0/3 | 0/3 | 3/3 | N/A | N/A | N/A | N/A | 30.45 |
| 34 | 0/3 | 0/3 | 0/3 | 0/3 | 3/3 | N/A | N/A | N/A | N/A | 30.42 |
| 35 | 0/3 | 3/3 | 0/3 | 0/3 | 3/3 | N/A | 34.9 | N/A | N/A | 30.37 |
| 36A | 0/3 | 0/3 | 0/3 | 0/3 | 3/3 | N/A | N/A | N/A | N/A | 30.65 |
| 36B | 3/3 | 0/3 | 0/3 | 0/3 | 3/3 | 34.7 | N/A | N/A | N/A | 30.54 |
| 37 | 3/3 | 0/3 | 0/3 | 0/3 | 3/3 | 23.9 | N/A | N/A | N/A | 30.50 |
| 38 | 0/3 | 0/3 | 0/3 | 0/3 | 3/3 | N/A | N/A | N/A | N/A | 30.63 |
| 39 | 0/3 | 0/3 | 0/3 | 0/3 | 3/3 | N/A | N/A | N/A | N/A | 30.60 |
| 40 | 0/4 | 0/4 | 0/4 | 0/4 | 4/4 | N/A | N/A | N/A | N/A | 31.67 |
| 41 | 0/3 | 0/3 | 0/3 | 0/3 | 3/3 | N/A | N/A | N/A | N/A | 31.24 |
| 42 | 0/3 | 0/3 | 0/3 | 0/3 | 3/3 | N/A | N/A | N/A | N/A | 30.56 |
| 43 | 0/3 | 0/3 | 0/3 | 0/3 | 3/3 | N/A | N/A | N/A | N/A | 30.73 |
| 44 | 0/3 | 0/3 | 0/3 | 0/3 | 3/3 | N/A | N/A | N/A | N/A | 30.64 |

TABLE 9
Cross-reactivity Supplemental Results

| | N positive/N tested | | | | | Average CT | | | | |
|---|---|---|---|---|---|---|---|---|---|---|
| | FAM | HEX | ROX | Q670 | Q705 | FAM | HEX | ROX | Q670 | Q705 |
| Organism | (Crypto) | (E hist) | (Giardia) | (Cyclo) | (IC) | (Crypto) | (E hist) | (Giardia) | (Cyclo) | (IC) |
| Necator | 0/5 | 0/5 | 0/5 | 0/5 | 5/5 | N/A | N/A | N/A | N/A | 30.93 |
| americanus | | | | | | | | | | |
| Ancylostoma | 0/3 | 0/3 | 0/3 | 0/3 | 3/3 | N/A | N/A | N/A | N/A | 30.55 |
| duodenale | | | | | | | | | | |
| Entamoeba | 0/3 | 3/3 | 0/3 | 0/3 | 3/3 | N/A | 35.8 | N/A | N/A | 30.80 |
| nuttalli | | | | | | | | | | |
| Entamoeba | 0/3 | 0/3 | 0/3 | 0/3 | 3/3 | N/A | N/A | N/A | N/A | 30.47 |
| bangladeshi | | | | | | | | | | |
| Entamoeba | 0/3 | 0/3 | 0/3 | 0/3 | 3/3 | N/A | N/A | N/A | N/A | 30.62 |
| hartmanni | | | | | | | | | | |
| Cryptosporidium | 0/3 | 0/3 | 0/3 | 0/3 | 3/3 | N/A | N/A | N/A | N/A | 30.39 |
| canis | | | | | | | | | | |
| Cryptosporidium | 3/3 | 0/3 | 0/3 | 0/3 | 3/3 | 31.7 | N/A | N/A | N/A | 30.55 |
| meleagridis | | | | | | | | | | |
| Cryptosporidium | 3/3 | 0/3 | 0/3 | 0/3 | 3/3 | 23.1 | N/A | N/A | N/A | 30.50 |
| baileyi | | | | | | | | | | |
| Cryptosporidium | 3/3 | 0/3 | 0/3 | 0/3 | 3/3 | 31.7 | N/A | N/A | N/A | 30.64 |
| ubiquitum | | | | | | | | | | |
| Cryptosporidium | 3/3 | 0/3 | 0/3 | 0/3 | 3/3 | 23.8 | N/A | N/A | N/A | 30.60 |
| wrairi | | | | | | | | | | |
| Cryptosporidium | 0/3 | 0/3 | 0/3 | 0/3 | 3/3 | N/A | N/A | N/A | N/A | 30.64 |
| muris | | | | | | | | | | |

The assay originally was designed to detect only Cryptosporidium parvum and Cryptosporidium hominis. Given this cross-reactivity with other Cryptosporidium species and the fact that all can infect humans, it was determined that 10 Cryptosporidium species will be intended targets of the assay (including C. hominis, C. parvum, and the four Cryptosporidium species shown as cross-reactive in this Example). Therefore, the only cross-reactive species is Entamoeba nutalli, and Cryptosporidium species will be tested as part of Inclusivity and not Specificity.

### Example 4: Evaluation of Oligonucleotides for G. lamblia and E. histolytica Inclusivity

The primer and probes shown in Table 3 (see Example 2) were tested in multiplex for inclusivity against different strains of Giardia lamblia and Entamoeba histolytica. G. lamblia strains were tested at 1.5 cells/mL in Aptima® tube and E. histolytica strains were tested at 0.006 cells/mL (3×LoD; LoD values of G. lamblia and E. histolytica at the time of inclusivity testing were 0.5 cells/mL and 0.002 cells/mL, respectively). Results are shown in Tables 10 below. Some strains of E. histolytica were not detected at 3×LoD, so were re-tested at higher concentrations (results shown in Table 11 below).

TABLE 10
Giardia and E. histolytica Inclusivity Results

| | | | | | | | Entamoeba | | | | | | Cyclospora | | |
|---|---|---|---|---|---|---|---|---|---|---|---|---|---|---|---|
| | | | | Cryptosporidium | | | histolytica | | | | | | cayetanensis | | |
| | | | IC | (FAM) | | | (HEX) | | | Giardia (ROX) | | | (Quasar 670) | | |
| | | Testing | Valid | Reac- | Avg | SD | Reac- | Avg | SD | Reac- | Avg | SD | Reac- | Avg | SD |
| Organism | ATCC/Strain | Conc. | N | tivity | Ct | Ct | tivity | Ct | Ct | tivity | Ct | Ct | tivity | Ct | Ct |
| Giardia | ATCC PRA-249 | 3X LoD | 3/3 | 0/3 | — | — | 0/3 | — | — | 3/3 | 32.16 | 0.40 | 0/3 | — | — |
| | ATCC PRA-245 | 3X LoD | 3/3 | 0/3 | — | — | 0/3 | — | — | 3/3 | 32.94 | 0.95 | 0/3 | — | — |
| | ATCC PRA-248 | 3X LoD | 3/3 | 0/3 | — | — | 0/3 | — | — | 3/3 | 31.48 | 0.38 | 0/3 | — | — |
| | ATCC PRA-252 | 3X LoD | 3/3 | 0/3 | — | — | 0/3 | — | — | 3/3 | 32.32 | 0.33 | 0/3 | — | — |
| | ATCC PRA-254 | 3X LoD | 3/3 | 0/3 | — | — | 0/3 | — | — | 3/3 | 32.35 | 0.15 | 0/3 | — | — |
| E. | ATCC 30015 | 3X LoD | 3/3 | 0/3 | — | — | 3/3 | 38.58 | 1.41 | 0/3 | — | — | 0/3 | — | — |
| histolytica | ATCC 30190 | 3X LoD | 3/3 | 0/3 | — | — | 3/3 | 32.91 | 0.32 | 0/3 | — | — | 0/3 | — | — |
| | ATCC 30889 | 3X LoD | 3/3 | 0/3 | — | — | 3/3 | 37.97 | 1.41 | 0/3 | — | — | 1/3 | 39.74 | — |
| | ATCC 50007 | 3X LoD | 3/3 | 0/3 | — | — | 1/3 | 39.19 | — | 0/3 | — | — | 0/3 | — | — |
| | ATCC 30887 | 3X LoD | 3/3 | 0/3 | — | — | 1/3 | 39.05 | — | 0/3 | — | — | 0/3 | — | — |
| | ATCC 30459 | 3X LoD | 3/3 | 0/3 | — | — | 1/3 | 39.40 | — | 0/3 | — | — | 0/3 | — | — |
| | ATCC 30458 | 3X LoD | 3/3 | 0/3 | — | — | 1/3 | 39.51 | — | 0/3 | — | — | 0/3 | — | — |
| | ATCC 50738 | 3X LoD | 3/3 | 0/3 | — | — | 2/3 | 38.06 | 1.67 | 0/3 | — | — | 0/3 | — | — |
| | ATCC PRA-357 | 3X LoD | 3/3 | 0/3 | — | — | 0/3 | — | — | 0/3 | — | — | 0/3 | — | — |
| | ATCC PRA-352 | 3X LoD | 3/3 | 0/3 | — | — | 2/3 | 37.88 | 0.12 | 0/3 | — | — | 0/3 | — | — |
| | ATCC 30457 | 3X LoD | 3/3 | 0/3 | — | — | 2/3 | 39.83 | 0.54 | 0/3 | — | — | 0/3 | — | — |
| | ATCC PRA-373 | 3X LoD | 3/3 | 0/3 | — | — | 0/3 | — | — | 0/3 | — | — | 0/3 | — | — |
| | ATCC 30885 | 3X LoD | 3/3 | 0/3 | — | — | 3/3 | 37.60 | 0.33 | 0/3 | — | — | 0/3 | — | — |
| | ATCC 30922 | 3X LoD | 3/3 | 0/3 | — | — | 0/3 | — | — | 0/3 | — | — | 0/3 | — | — |
| | ATCC 30891 | 3X LoD | 3/3 | 0/3 | — | — | 3/3 | 36.45 | 0.55 | 0/3 | — | — | 0/3 | — | — |

TABLE 11
E. hisolytica Inclusivity Results at Higher Target Concentrations

| | | | | Cryptosporidium | | | Entamoeba histolytica | | | | | | Cyclospora cayetanensis | | |
|---|---|---|---|---|---|---|---|---|---|---|---|---|---|---|---|
| | | | IC | (FAM) | | | (HEX) | | | Giardia (ROX) | | | (Quasar 670) | | |
| | | Testing | Valid | Reac- | Avg | SD | | Avg | SD | Reac- | Avg | SD | | Avg | SD |
| Organism | ATCC/Strain | Conc. | N | tivity | Ct | Ct | Reactivity | Ct | Ct | tivity | Ct | Ct | Reactivity | Ct | Ct |
| E. | ATCC 50007 | 6x LoD | 3/3 | 0/3 | — | — | 1/3 | 36.42 | — | 0/3 | — | — | 0/3 | — | — |
| histolytica | | 10x LoD | 3/3 | 0/3 | — | — | 3/3 | 37.27 | 0.26 | 0/3 | — | — | 0/3 | — | — |
| | | 50x LoD | 3/3 | 0/3 | — | — | 3/3 | 35.57 | 0.16 | 0/3 | — | — | 0/3 | — | — |
| | ATCC 30887 | 6x LoD | 3/3 | 0/3 | — | — | 2/3 | 39.96 | 0.54 | 0/3 | — | — | 0/3 | — | — |
| | | 10x LoD | 3/3 | 0/3 | — | — | 2/3 | 38.23 | 1.15 | 0/3 | — | — | 0/3 | — | — |
| | | 50x LoD | 3/3 | 0/3 | — | — | 3/3 | 34.39 | 0.54 | 0/3 | — | — | 0/3 | — | — |
| | ATCC 30459 | 50x LoD | 3/3 | 0/3 | — | — | 3/3 | 35.77 | 1.05 | 0/3 | — | — | 0/3 | — | — |
| | ATCC 30458 | 6x LoD | 3/3 | 0/3 | — | — | 2/3 | 37.66 | 0.86 | 0/3 | — | — | 0/3 | — | — |
| | | 10x LoD | 3/3 | 0/3 | — | — | 3/3 | 36.34 | 0.24 | 0/3 | — | — | 0/3 | — | — |
| | | 50x LoD | 3/3 | 0/3 | — | — | 3/3 | 34.98 | 0.46 | 0/3 | — | — | 0/3 | — | — |
| | ATCC 50738 | 6x LoD | 3/3 | 0/3 | — | — | 3/3 | 38.02 | 1.75 | 0/3 | — | — | 0/3 | — | — |
| | ATCC PRA-357 | 50x LoD | 3/3 | 0/3 | — | — | 3/3 | 36.64 | 0.44 | 0/3 | — | — | 0/3 | — | — |
| | ATCC PRA-352 | 50x LoD | 3/3 | 0/3 | — | — | 3/3 | 34.92 | 0.23 | 0/3 | — | — | 0/3 | — | — |
| | ATCC 30457 | 6x LoD | 3/3 | 0/3 | — | — | 3/3 | 38.45 | 1.54 | 0/3 | — | — | 0/3 | — | — |
| | ATCC PRA-373 | 50x LoD | 3/3 | 0/3 | — | — | 3/3 | 35.68 | 0.29 | 0/3 | — | — | 0/3 | — | — |
| | ATCC 30922 | 50x LoD | 3/3 | 0/3 | — | — | 1/3 | 37.90 | — | 0/3 | — | — | 0/3 | — | — |
| | | 1 cell/mL | 3/3 | 0/3 | — | — | 3/3 | 34.75 | 0.30 | 0/3 | — | — | 0/3 | — | — |

### Example 5: Evaluation of Oligonucleotides for Cyclosporidium Spp. Inclusivity

The primers and probes shown in Table 3 (see Example 2) were tested in multiplex for inclusivity against different Cryptosporidium species. Gblocks ordered from IDT were made into plasmids, and plasmids were tested at 1E6 cp/mL. Results are shown in Table 12 below.

TABLE 12
Cryptosporidium Inclusivity Results

| Plasmid | FAM channel (Cryptosporidium) | | | | |
|---|---|---|---|---|---|
| (1E6 cp/mL) | Reactivity | Avg Ct | SD Ct | Avg RFU | SD RFU |
| C. andersoni | 0/3 | NA | NA | −20 | 9 |
| C. baileyi | 3/3 | 23.8 | 0.2 | 27505 | 2682 |
| C. canis | 0/3 | NA | NA | 23 | 93 |
| C. felis | 3/3 | 25.6 | 0.1 | 9907 | 307 |
| C. meleagridis | 3/3 | 25.9 | 0.1 | 9004 | 298 |
| C. muris | 0/3 | NA | NA | −35 | 7 |
| C. parvum | 3/3 | 23.9 | 0.1 | 33008 | 1995 |
| C. ubiquitum | 3/3 | 26.1 | 0.1 | 8388 | 675 |
| C. wrairi | 3/3 | 23.5 | 0.0 | 32995 | 741 |

Sequence analysis predicts that the assay will also detect C. cuniculus, C. viatorum, and C. tyzzeri.

### Example 6: Giardia lamblia Primer and Probe Set Comparison

A Giardia primer and probe set targeting the B-Giardin gene was compared against a second Giardia oligo set targeting the 18S rRNA gene. The primers and probes used in the experiment are shown in Table 13, and results are shown in Table 14 below.

TABLE 13

| | Oligo | SEQ ID | |
|---|---|---|---|
| Target | Type | NO | Modifications |
| G. lamblia | Primer | 5 | |
| B-Giardin | Primer | 6 | 5 mC at residues 4 and 6 |
| | Primer | 29 | |
| | Probe | 32 | 5 mC at residues 4, 7, 13, 14, 16, and 19 |
| | | | CalOrange560/BHQ1 |
| G. lamblia | Primer | 25 | 5 mC at residues 1, 4, 6, and 8 |
| 18S rRNA | Primer | 3 | |
| | Probe | 41 | 5 mC at residues 7, 11, 15, and 20 |
| | | | CalRed610/BHQ2 |

TABLE 14

| | Giardia B-Giardin | | | Giardia 18s | | |
|---|---|---|---|---|---|---|
| Sample | Pos | Avg Ct | SD Ct | Pos | Avg Ct | SD Ct |
| NYBio 29 | 0/1 | NA | NA | 1/1 | 34.1 | NA |
| NYBio 74_1x | 1/1 | 23.0 | NA | 1/1 | 23.5 | NA |
| MultiOrg_L_CBSSTM^{†} | 0/5 | NA | NA | 3/5 | 36.3 | 0.4 |
| MultiOrg_H_CBSSTM^{‡} | 5/5 | 36.0 | 1.1 | 5/5 | 30.7 | 0.1 |

^{†}MultiOrg_L_CBSSTM = Multitarget organisms spiked Low concentration in Cary Blair Stool in STM Sample Transport Medium; 3% (w/v) lithium lauryl sulfate (LLS), 0.2% (w/v) NaH2PO4, 0.2% (w/v) Na2HPO4, 0.04% (w/v) EDTA, 0.04% EGTA (w/v), pH 6.7).
^{‡}MultiOrg_H_CBSSTM = Multitarget organisms spiked High concentration in Cary Blair Stool in STM.
Low concentration = 10 cells/mL of C. parvum. High concentration = 300 cells/mL of C. parvum.

### Example 7: Evaluation of Cryptosporidium Oligonucleotides

Primer and probe sets targeting the 18S rRNA gene of Cryptosporidium spp. were evaluated in multiplex versions of the assay for cross-reactivity to Cyclospora cayetanensis.

### Study 1

One study compared the two Cryptosporidium primers and probe sets shown in Table 15 below.

TABLE 15

| Oligo | Oligo | SEQ ID | |
|---|---|---|---|
| Set | Type | NO | Modifications |
| 1 | Primer | 33 | 5 mC at residues 3 and 11 |
| | Primer | 28 | 5 mC at residues 1, 7, and 11-13 |
| | Probe | 24 | 5 mC at residues 2, 6, 9, and 16 |
| | | | pdU at residues 5, 19, and 26 |
| | | | FAM/BHQ1 |
| | | | Reverse polarity C |
| 2 | Primer | 4 | |
| | Primer | 37 | 5 mC at residues 2, 5, 7, 11, and 12 |
| | Probe | 48 | 5 mC at residues 2, 6, 9, and 19 |
| | | | pdU at residues 3, 5, 10, 20, 22, 24, and 26 |
| | | | FAM/BHQ1 |
| | | | Reverse polarity C |

Results are shown in Table 16 below. Both oligo sets cross-reacted with Cyclospora as Cyclospora plasmid was detected in the FAM channel (the channel for Cryptosporidium). These two sets of oligos were disregarded due to their cross-reactivity with Cyclospora target.

TABLE 16
FAM Channel (Cryptosporidium)

| | Sample | | | | | | |
|---|---|---|---|---|---|---|---|
| | concen- | | | | | | |
| | tration | | | | | | |
| | (copies/ | Oligo | Reac- | Avg | SD | Avg | |
| Sample | mL) | Set* | tivity | Ct | Ct | RFU | SD RFU |
| Cyclospora | 1E8 | 1 | 7/9 | 39.94 | 2.45 | 5544.33 | 4768.17 |
| Plasmid | | 2 | 9/9 | 32.67 | 0.26 | 4133.44 | 376.90 |
| | 1E7 | 1 | 2/9 | 42.97 | 0.38 | 401.78 | 682.02 |
| | | 2 | 9/9 | 35.72 | 0.47 | 3279.89 | 537.79 |
| | 1E6 | 1 | 1/9 | 38.40 | NA | 1107.89 | 3338.42 |
| | | 2 | 9/9 | 39.00 | 0.66 | 2328.11 | 470.72 |
| | 1ES | 1 | 0/9 | NA | NA | NA | NA |
| | | 2 | 7/9 | 43.17 | 1.18 | 1134.89 | 339.43 |
| | 1E4 | 1 | 0/9 | NA | NA | NA | NA |
| | | 2 | 0/9 | NA | NA | NA | NA |

*Cryptosporidium oligo set (from Table 15) in a multiplex version of the assay also targeting E. histolytica, G. lamblia, and C. cayetanensis.

### Study 2

In another study, a new set of Cryptosporidium oligonucleotides targeting the 18S rRNA gene were compared against Oligo Set 2 from Table 15. This new set (Oligo Set 3) is shown in Table 17 below.

TABLE 17

| Oligo | Oligo | SEQ ID | |
|---|---|---|---|
| Set | Type | NO | Modifications |
| 3 | Primer | 11 | 5 mC at residues 3, 4, 7, and 16 |
| | | | pdU at residues 8, 12, and 18 |
| | Primer | 49 | 5 mC at residues 3, 4, 10, and 13-15 |
| | | | pdU at residues 1, 5, 11, 18, and 21 |
| | Probe | 48 | 5 mC at residues 2, 6, 9, and 19 |
| | | | pdU at residues 3, 5, 10, 20, 22, 24, and 26 |
| | | | FAM/BHQ1 |
| | | | Reverse polarity C |

Cyclospora plasmid at 1E9 cp/mL was tested with two versions of the GI Parasite multiplex, each with a different set of Cryptosporidium oligos (Set 2 shown in Table 15 or Set 3 shown in Table 17). Results are shown in Table 18 below.

TABLE 18

| | Oligo | FAM Channel (Cryptosporidium) | | | | |
|---|---|---|---|---|---|---|
| Sample | Set* | Reactivity | Avg Ct | SD Ct | Avg RFU | SD RFU |
| 100 cells/mL C. parvum | 3 | 4/4 | 22.7 | 0.3 | 49967 | 2092 |
| in STM† | 2 | 4/4 | 23.3 | 0.2 | 43143 | 1371 |
| 100 cells/mL C. parvum | 3 | 4/4 | 22.8 | 0.3 | 51753 | 1806 |
| in STM† + HeLa | 2 | 4/4 | 23.4 | 0.2 | 44450 | 1054 |
| Cyclospora plasmid in | 3 | 0/4 | NA | NA | −7 | 5 |
| STM† | 2 | 4/4 | 38.3 | 0.8 | 1792 | 261 |
| Cyclospora plasmid in | 3 | 0/4 | NA | NA | −9 | 2 |
| STM† + HeLa | 2 | 3/4 | 39.7 | 1.0 | 1273 | 330 |
| | Oligo | ROX Channel (Giardia) | | | | |
| Sample | Set* | Reactivity | Avg Ct | SD Ct | Avg RFU | SD RFU |
| 25 cells/mL Giardia in | 3 | 4/4 | 31.4 | 0.2 | 174950 | 10302 |
| STM† + HeLa | 2 | 4/4 | 30.8 | 0.5 | 233637 | 23614 |
| 25 cells/mL Giardia in | 3 | 4/4 | 31.5 | 0.6 | 176256 | 19306 |
| STM† | 2 | 4/4 | 30.6 | 0.3 | 241180 | 5349 |

*Cryptosporidium oligo set (from Tables 15 and 17) in a multiplex version of the assay also targeting E. histolytica, G. lamblia, and C. cayetanensis.
†Sample Transport Medium; 3% (w/v) lithium lauryl sulfate (LLS), 0.2% (w/v) NaH2PO4, 0.2% (w/v) Na2HPO4, 0.04% (w/v) EDTA, 0.04% EGTA (w/v), pH 6.7.

The new oligos (Set 3) did not cross-react with Cyclospora and had no impact to Cryptosporidium sensitivity. However, with the new Cryptosporidium oligos in the multiplex assay, there was a minor impact to Giardia sensitivity (higher ROX Ct and lower ROX RFU).

### Study 3

The Cryptosporidium reverse primer from Set 3 was re-designed and tested in a third study. The re-designed reverse primer nucleotide sequence is SEQ ID NO:37 (with 5mC at residues 2, 5, 7, 11, and 12).

Four different versions of the GI parasite assay multiplex were evaluated, each containing a different set of Cryptosporidium oligonucleotides. Relative to the Set 3 oligos, these four different sets of Crypto oligos are as follows: removing the forward primer (Set 3.1), replacing the Set 3 reverse primer with the re-designed reverse primer (Set 3.2), removing the Set 3 reverse primer (Set 3.3), and keeping all three of the original Set 3 oligos (Set 3.4). These combinations are also summarized in Table 19 below.

TABLE 19
Cryptosporidium Oligo Sets for Third
Study (with Reference to Oligo Set 3)

| | Set 3.1 | Set 3.2 | Set 3.3 | Set 3.4 |
|---|---|---|---|---|
| Forward Primer (FR) | None | Set 3 FR | Set 3 FR | Set 3 FR |
| Reverse Primer (RP) | Set 3 RP | Re-designed | None | Set 3 RP |
| | | RP* | | |
| Probe (PR) | Set 3 PR | Set 3 PR | Set 3 PR | Set 3 PR |

*SEQ ID NO: 37 (with 5 mC at residues 2, 5, 7, 11, and 12)

The results of this experiment are shown in Table 20 below.

TABLE 20

| | Oligo | FAM channel (Cryptosporidium) | | | | |
|---|---|---|---|---|---|---|
| Sample ID | Set* | Hit Rate | Avg Ct | SD Ct | Avg RFU | SD RFU |
| 100 cells/mL | 3.1 | 0/4 | NA | NA | −10 | 8 |
| Crypto in | 3.2 | 4/4 | 23.5 | 0.1 | 47012 | 2520 |
| STM† | 3.3 | 3/4 | 38.0 | 0.5 | 9396 | 6657 |
| | 3.4 | 4/4 | 23.1 | 0.2 | 50718 | 477 |
| | Oligo | ROX channel (Giardia) | | | | |
| Sample ID | Set* | Hit Rate | Avg Ct | SD Ct | Avg RFU | SD RFU |
| 25 cells/mL | 3.1 | 4/4 | 31.5 | 0.4 | 182580 | 18270 |
| Giardia in | 3.2 | 4/4 | 31.5 | 0.3 | 259313 | 13611 |
| STM† | 3.3 | 4/4 | 31.8 | 0.7 | 241289 | 21637 |
| | 3.4 | 4/4 | 31.7 | 0.2 | 177990 | 10670 |

*Cryptosporidium oligo set (from Table 19) in a multiplex version of the assay also targeting E. histolytica, G. lamblia, and C. cayetanensis.
†Sample Transport Medium; 3% (w/v) lithium lauryl sulfate (LLS), 0.2% (w/v) NaH2PO4, 0.2% (w/v) Na2HPO4, 0.04% (w/v) EDTA, 0.04% EGTA (w/v), pH 6.7.

Conclusions: Removing the original Set 3 reverse primer (Set 3.3) or switching it for re-designed reverse primer (Set 3.2) increased Giardia RFU.

### Example 8: Evaluation of Entamoeba histolytica Oligonucleotides

Primer and probe sets targeting the 18S rRNA gene of Entamoeba histolytica were evaluated in multiplex versions of the assay with one retrospective clinical positive specimen from Cerba (Cerba 79) and with contrived specimens prepared with E. histolytica in vitro cell suspension (ATCC 30889) spiked at 600 cells/mL in indifferent negative Cary Blair Stool specimens (from Discovery Life Sciences).

The E. histolytica and G. lamblia oligonucleotides tested (without reference to multiplex combinations) are shown in Table 21 below.

TABLE 21

| Target | | | | |
|---|---|---|---|---|
| Organism and | Oligo Set | Oligo | SEQ | |
| Gene | ID | Type | ID NO | Modifications |
| E. histolytica | E. histo | Primer | 21 | |
| CP5 gene | CP5 | Primer | 20 | |
| | | Probe | 34 | 5 mC at residues 4, 10, and 16 |
| | | | | pdU at residues 2, 5, 7, 17, and 21 |
| | | | | HEX/BHQ1 |
| | | | | Reverse polarity C |
| E. histolytica | E. histo | Primer | 36 | |
| 18S rRNA gene | 18S Set 1 | Primer | 43 | |
| | | Probe | 7 | 5 mC at residues 2, 10, 11, 16, and 20 |
| | | | | pdU at residues 7, 14, 15, 18, 19, 23, 27, and 28 |
| | | | | CalOrange560/BHQ1 |
| | E. histo | Primer | 42 | |
| | 18S Set 2 | Primer | 19 | 5 mC at residues 1, 9, 11, and 20 |
| | | | | pdU at residues 7, 13, 17, and 22 |
| | | Probe | 44 | 5 mC at residues 14, 15, 17, and 20 |
| | | | | pdU at residues 7, 12, 23, and 24 |
| | | | | CalOrange560/BHQ1 |
| G. lamblia | Giardia | Primer | 5 | |
| bG gene | bG | Primer | 6 | 5 mC at residues 4 and 6 |
| | | Primer | 29 | |
| | | Probe | 32 | 5 mC at residues 4, 7, 13, 14, 16, and 19 |
| | | | | CalRed610/BHQ2 |
| G. lamblia | Giardia | Primer | 25 | 5 mC at residues 1, 4, 6, and 8 |
| 18S rRNA gene | 18S | Primer | 3 | |
| | | Probe | 41 | 5 mC at residues 7, 11, 15, and 20 |
| | | | | CalRed610/BHQ2 |

Multiplex combinations (“oligo mixes”) of E. histolytica and Giardia oligonucleotide sets from Table 21 are shown in Table 22 below.

TABLE 22

| | Oligo Mix 1* | Oligo Mix 2* | Oligo Mix 3* | Oligo Mix 4* |
|---|---|---|---|---|
| E. histolytica | E. histo | E. histo | E. histo | E. histo |
| Oligo Set | CP5 | 18S Set 1 | 18S Set 2 | 18S Set 2 |
| G. lamblia | Giardia | Giardia | Giardia | Giardia |
| Oligo Set | bG | 18S | bG | 18S |

*All four oligo mixes were prepared with C. cayetanensis and Cryptosporidium spp. oligos and tested in multiplex

Results are shown in Table 23 below.

TABLE 23

| | | HEX | | | Quasar 705 | | |
|---|---|---|---|---|---|---|---|
| | | | Aver- | Aver- | | Aver- | Aver- |
| Sample | Oligo | Hit | age | age | Hit | age | age |
| Type | Mix | Rate | Ct | RFU | Rate | Ct | RFU |
| Neg Ctrl | 2 | 0 | | −98 | 1 | 26.3 | 30003 |
| | 4 | 0 | | −19 | 1 | 26.6 | 27926 |
| | 3 | 0 | | 44 | 1 | 26.4 | 28124 |
| | 1 | 0 | | −34 | 1 | 26.5 | 28661 |
| Contrived | DLS-286 | | | | | | |
| | 2 | 9 | 23.9 | 45528 | 9 | 26.4 | 29098 |
| | 4 | 9 | 22.1 | 128959 | 9 | 26.5 | 28861 |
| | 3 | 9 | 22.0 | 132519 | 9 | 26.2 | 29166 |
| | 1 | 9 | 30.5 | 226884 | 9 | 26.4 | 29127 |
| | DLS-290 | | | | | | |
| | 2 | 9 | 23.2 | 47408 | 9 | 26.6 | 29567 |
| | 4 | 9 | 21.3 | 130520 | 9 | 26.9 | 28870 |
| | 3 | 9 | 21.3 | 130757 | 9 | 26.6 | 28655 |
| | 1 | 9 | 30.0 | 230918 | 9 | 26.6 | 29341 |
| | DLS-291 | | | | | | |
| | 2 | 9 | 30.0 | 38289 | 9 | 26.3 | 28372 |
| | 4 | 9 | 27.4 | 125353 | 9 | 26.4 | 28970 |
| | 3 | 9 | 27.1 | 125819 | 9 | 26.2 | 28627 |
| | 1 | 5 | 36.8 | 32917 | 9 | 26.4 | 28964 |
| | DLS-292 | | | | | | |
| | 2 | 9 | 25.5 | 45426 | 9 | 26.4 | 29206 |
| | 4 | 9 | 23.7 | 127791 | 9 | 26.6 | 28808 |
| | 3 | 9 | 23.6 | 132321 | 9 | 26.3 | 29265 |
| | 1 | 9 | 31.8 | 206804 | 9 | 26.6 | 28504 |
| | DLS-295 | | | | | | |
| | 2 | 9 | 27.7 | 43813 | 9 | 26.5 | 29033 |
| | 4 | 9 | 25.9 | 125667 | 9 | 26.6 | 29052 |
| | 3 | 9 | 25.8 | 131957 | 9 | 26.2 | 29402 |
| | 1 | 9 | 32.8 | 185771 | 9 | 26.5 | 29194 |
| Natural | Cerba 79 | | | | | | |
| Positive | 2 | 9 | 29.2 | 34619 | 9 | 26.2 | 25906 |
| | 4 | 9 | 26.9 | 117101 | 9 | 26.3 | 25165 |
| | 3 | 9 | 26.8 | 121384 | 9 | 26.1 | 26703 |
| | 1 | 9 | 36.2 | 38364 | 9 | 26.4 | 26461 |

One of the two sets of oligos designed on the 18S gene of E. histolytica (E. histolytica 18S Set 1) had worse sensitivity than the other one (E. histolytica 18S Set 2). The E. histolytica 18S Set 2 oligos performed best.

### Example 9: Evaluation of Cyclospora cayetanensis Oligonucleotides

Primer and probe sets targeting Cyclospora cayetanensis were evaluated (in duplex with IC oligos). Serial dilutions of Cyclospora plasmid were tested with and without HeLa cells to check impact to sensitivity when in presence of human genomic DNA.

The Cyclospora oligonucleotides tested are shown in Table 24 below.

TABLE 24

| | | SEQ ID | |
|---|---|---|---|
| Oligo Set | Oligo Type | NO | Modifications |
| 1 | Primer | 23 | |
| | Primer | 18 | |
| | Probe | 12 | Quasar670/BHQ2 |
| 2 | Primer | 31 | 5 mC at residues 10 and 14 |
| | Primer | 22 | 5 mC at residue 3 |
| | Probe | 10 | Quasar670/BHQ2 |
| 3 | Primer | 35 | 5 mC at residues 1, 5, and 18 |
| | Primer | 13 | 5 mC at residues 3, 8, 11, and 14 |
| | Probe | 47 | 5 mC at residues 2 and 3 |
| | | | Quasar670/BHQ2 |
| 4 | Primer | 38 | |
| | Primer | 51 | 5 mC at residues 5, 6, 19, and 20 |
| | Probe | 30 | 5 mC at residues 9, 12, and 19 |
| | | | Quasar670/BHQ2 |
| 5 | Primer | 26 | 5 mC at residues 10 and 11 |
| | Primer | 14 | 5 mC at residues 8 and 10 |
| | Probe | 45 | 5 mC at residues 7-9, 23, 25, and 28 |
| | | | pdU at residue 5 |
| | | | Quasar670/BHQ2 |
| 6 | Primer | 1 | 5 mC at residues 5, 6, and 9 |
| | Primer | 9 | 5 mC at residue 2 |
| | Probe | 50 | 5 mC at residue 6 |
| | | | Quasar670/BHQ2 |

Results (values for Ct and RFU in Q670 channel for Cyclospora) are shown in Table 25 below.

TABLE 25

| | | + HeLa | | | | | No HeLa | | | | |
|---|---|---|---|---|---|---|---|---|---|---|---|
| Oligo | Conc. | Count | Avg | SD | Avg | SD | Count | Avg | SD | Avg | SD |
| Set | (cp/mL) | of Ct | Ct | Ct | RFU | RFU | of Ct | Ct | Ct | RFU | RFU |
| 1 | 3E1 | 0 | NA | NA | −2 | 1 | 3 | 35.7 | 0.4 | 1563 | 90 |
| | 3E2 | 3 | 38.4 | 3.6 | 225 | 27 | 3 | 33.0 | 0.1 | 1899 | 138 |
| | 3E3 | 3 | 28.8 | 0.2 | 1088 | 37 | 3 | 29.4 | 0.1 | 2574 | 208 |
| | 3E4 | 3 | 26.2 | 0.1 | 1320 | 73 | 3 | 26.2 | 0.3 | 2755 | 291 |
| | 3E5 | 3 | 22.3 | 0.1 | 2294 | 82 | 3 | 22.7 | 0.1 | 3133 | 85 |
| | NTC | 0 | NA | NA | −1 | 1 | 0 | NA | NA | −1 | 1 |
| 2 | 3E1 | 3 | 34.1 | 0.1 | 2359 | 157 | 3 | 34.1 | 0.1 | 2902 | 168 |
| | 3E2 | 3 | 32.1 | 0.0 | 1948 | 34 | 3 | 31.4 | 0.1 | 3089 | 45 |
| | 3E3 | 3 | 28.3 | 0.1 | 2934 | 151 | 3 | 28.5 | 0.1 | 3206 | 212 |
| | 3E4 | 3 | 25.0 | 0.2 | 2666 | 270 | 3 | 24.7 | 0.2 | 3625 | 285 |
| | 3E5 | 3 | 21.7 | 0.1 | 2779 | 178 | 3 | 21.4 | 0.1 | 3390 | 89 |
| | NTC | 3 | 37.0 | 1.1 | 1094 | 259 | 3 | 37.8 | 0.4 | 1263 | 106 |
| 3 | 3E1 | 3 | 32.3 | 0.5 | 1651 | 103 | 3 | 34.6 | 0.1 | 1987 | 104 |
| | 3E2 | 3 | 31.1 | 0.3 | 1829 | 197 | 3 | 31.8 | 0.1 | 2592 | 172 |
| | 3E3 | 3 | 28.1 | 0.1 | 2702 | 177 | 3 | 28.4 | 0.2 | 3121 | 295 |
| | 3E4 | 3 | 24.9 | 0.1 | 3109 | 47 | 3 | 25.2 | 0.0 | 3020 | 39 |
| | 3E5 | 3 | 21.8 | 0.2 | 3485 | 334 | 3 | 21.5 | 0.1 | 3711 | 160 |
| | NTC | 3 | 33.2 | 0.7 | 1268 | 243 | 3 | 36.8 | 0.3 | 1567 | 115 |
| 4 | 3E1 | 0 | NA | NA | 4 | 14 | 0 | NA | NA | 12 | 13 |
| | 3E2 | 3 | 33.8 | 0.6 | 615 | 140 | 3 | 34.6 | 1.1 | 660 | 180 |
| | 3E3 | 3 | 31.6 | 0.9 | 710 | 134 | 3 | 29.3 | 0.2 | 1760 | 141 |
| | 3E4 | 3 | 26.5 | 0.5 | 1345 | 187 | 3 | 28.2 | 0.9 | 1013 | 118 |
| | 3E5 | 3 | 23.0 | 0.2 | 1590 | 98 | 3 | 23.4 | 0.8 | 1585 | 274 |
| | NTC | 0 | NA | NA | 4 | 6 | 0 | NA | NA | −3 | 2 |
| 5 | 3E1 | 3 | 36.1 | 0.4 | 1505 | 186 | 3 | 35.9 | 0.3 | 1618 | 125 |
| | 3E2 | 3 | 32.0 | 0.3 | 2580 | 141 | 3 | 32.2 | 0.2 | 2318 | 197 |
| | 3E3 | 3 | 28.6 | 0.1 | 2697 | 87 | 3 | 28.3 | 0.2 | 3043 | 212 |
| | 3E4 | 3 | 25.2 | 0.2 | 3172 | 400 | 3 | 25.1 | 0.3 | 3069 | 405 |
| | 3E5 | 3 | 21.5 | 0.0 | 3945 | 66 | 3 | 21.3 | 0.1 | 3757 | 137 |
| | NTC | 0 | NA | NA | 7 | 7 | 0 | NA | NA | −2 | 1 |
| 6 | 3E1 | 3 | 34.7 | 0.4 | 1446 | 82 | 3 | 34.9 | 0.4 | 1433 | 116 |
| | 3E2 | 3 | 31.3 | 0.2 | 2086 | 211 | 3 | 31.3 | 0.2 | 2265 | 262 |
| | 3E3 | 3 | 27.7 | 0.1 | 2811 | 50 | 3 | 28.0 | 0.1 | 2884 | 233 |
| | 3E4 | 3 | 24.6 | 0.2 | 3180 | 234 | 3 | 24.6 | 0.2 | 2922 | 222 |
| | 3E5 | 3 | 21.2 | 0.1 | 3561 | 85 | 3 | 21.2 | 0.1 | 3500 | 73 |
| | NTC | 0 | NA | NA | −1 | 1 | 0 | NA | NA | 0 | 0 |

Oligos were disregarded if they had higher Ct or lower RFU in the presence of HeLa cells (human gDNA) (Oligo Sets 1, 2, and 3). Oligos were disregarded if they had poor sensitivity (Oligo Set 4). Of Oligo Sets 5 and 6. Set 5 had higher RFU so it was selected to move forward.

### Sequences

TABLE 26
Exemplary Oligonucleotide Sequences

| SEQ | | | |
|---|---|---|---|
| ID NO | Sequence (5′→3′) | Target species | Function |
| 1 | AAATCCTTCCAGAGTAACAAT | Cyclospora cayetanensis | amplification oligomer |
| 2 | AACCGTTGTCCTGAGCCG | Giardia lamblia | amplification oligomer |
| 3 | AACCGTTGTCCTGAGCCGTC | Giardia lamblia | amplification oligomer |
| 4 | AACGAGACCTTAACCTGCTAAA | Cryptosporidium spp. | amplification oligomer |
| 5 | AAGCTCAGCAACATGAACC | Giardia lamblia | amplification oligomer |
| 6 | AAGCTCAGCAACATGAAGG | Giardia lamblia | amplification oligomer |
| 7 | ACAAAATGGCCAATTCATTCAATGAATTGA | Entamoeba histolytica | detection probe |
| 8 | ACCTGCTAAATAGACATAAGA | Cryptosporidium spp. | amplification oligomer |
| 9 | ACTGCAACAACTCTAATATACAC | Cyclospora cayetanensis | amplification oligomer |
| 10 | AGACGAACTACTGCGAAAGCATTTGCCAA | Cyclospora cayetanensis | detection probe |
| 11 | AGCCTGCTAAATAGACATAAGA | Cryptosporidium spp. | amplification oligomer |
| 12 | AGGACCCTCCGATCGGTTGCA | Cyclospora cayetanensis | detection probe |
| 13 | ATCTAATCGTCTTCAAACCC | Cyclospora cayetanensis | amplification oligomer |
| 14 | ATTGTTACTCTGGAAGGATTT | Cyclospora cayetanensis | amplification oligomer |
| 16 | ATGCCCTGTATTGTTATTTCTCGTC | Cyclospora cayetanensis | detection probe |
| 17 | ATTCTAAGTGAGTTAGGATGCCACG | Entamoeba histolytica | detection probe |
| 18 | CACAGCTGGTCAGTCCAATGAG | Cyclospora cayetanensis | amplification oligomer |
| 19 | CAGAAATTCTCATTGGTTACTTG | Entamoeba histolytica | amplification oligomer |
| 20 | CAGATTCTGGTACATCTCCCCTAGA | Entamoeba histolytica | amplification oligomer |
| 21 | CCAATTATCAGTTGAAGGACCATTTG | Entamoeba histolytica | amplification oligomer |
| 22 | CCCTACTGTCGTTCTTGAT | Cyclospora cayetanensis | amplification oligomer |
| 23 | CCGATTACGTCCCTGCCCC | Cyclospora cayetanensis | amplification oligomer |
| 24 | CCTATCAGCTTTAGACGGTAGGGTATTGG | Cryptosporidium spp. | detection probe |
| 25 | CGACGCTCTCCCCAAGGA | Giardia lamblia | amplification oligomer |
| 26 | CGCAAATTACCCAATGAAAA | Cyclospora cayetanensis | amplification oligomer |
| 28 | CGGAATCGAACCCTAATTCCC | Cryptosporidium spp. | amplification oligomer |
| 29 | CGGACCTCGATCTCGTT | Giardia lamblia | amplification oligomer |
| 30 | CGGTGAAACTGCGAATGGCTCATTAAAAC | Cyclospora cayetanensis | detection probe |
| 31 | CGTATTTAACTGTCAGAGGTG | Cyclospora cayetanensis | amplification oligomer |
| 32 | CGTCAGCAGGTTCCACGACAAGA | Giardia lamblia | detection probe |
| 33 | CTCATAATAACTTTACGGATCACA | Cryptosporidium spp. | amplification oligomer |
| 34 | CTGCTATGACTGAGGCTGAATA | Entamoeba histolytica | detection probe |
| 35 | CTGTCAGAGGTGAAATTCTT | Cyclospora cayetanensis | amplification oligomer |
| 36 | CTTGAGACGATCCAGTTTGTATTAG | Entamoeba histolytica | amplification oligomer |
| 37 | GCATCACAGACCTGTTATTGCCTA | Cryptosporidium spp. | amplification oligomer |
| 38 | GCTTGTCTCAAAGATTAAGCC | Cyclospora cayetanensis | amplification oligomer |
| 39 | GGTGAGCGGGCATGCATGGCTT | Giardia lamblia | detection probe |
| 40 | GGTGCGCGGGCATGCATGGCTT | Giardia lamblia | detection probe |
| 41 | GGTGMGCGGGCATGCATGGCTT | Giardia lamblia | detection probe |
| 42 | GTACAAAATGGCCAATTCATTCAATG | Entamoeba histolytica | amplification oligomer |
| 43 | GTCGTGGCATCCTAACTCACTTAG | Entamoeba histolytica | amplification oligomer |
| 44 | GTGAGTTAGGATGCCACGACAATTG | Entamoeba histolytica | detection probe |
| 45 | TAAATGCCCTGTATTGTTATTTCTCGTCA | Cyclospora cayetanensis | detection probe |
| 46 | TACAAAATGGCCAATTCATTCAATG | Entamoeba histolytica | amplification oligomer |
| 47 | TCCTTGGCAAATGCTTTCGCAGTAGTTC | Cyclospora cayetanensis | detection probe |
| 48 | TCTGTCTTCTTAGAGGGACTTTGTATG | Cryptosporidium spp. | detection probe |
| 49 | TGCCTAAAACTTCCCTGTATTA | Cryptosporidium spp. | amplification oligomer |
| 50 | TGGTGCCAGCAGCCGC | Cyclospora cayetanensis | detection probe |
| 51 | TTATCCATGTAAAAGAGACCA | Cyclospora cayetanensis | amplification oligomer |
| 52 | TTATCTGTCTTCTTAGAGGGACTTTGTA | Cryptosporidium spp. | detection probe |

TABLE 27
Exemplary Nucleotide Modifications and Labels

| SEQ ID | | |
|---|---|---|
| NO: | Sequence (5′→3′) | Exemplary Nucleotide Modification(s) |
| 53 | ttatccatgtaaaagagacca | 5-methyl cytosine at residues 4, 5, 19, & 20 |
| 54 | tacaaaatggccaattcattcaatg | 5-methyl cytosine at residues 3 & 17 |
| 55 | tggtgccagcagccgc | 5-methyl cytosine at residue 6 |
| 56 | taaatgccctgtattgttatttctcgtca | 5-methyl cytosine at residues 7-9, 23, 25, & 28 |
| | | Propyne dU at residue 5 |
| 57 | tccttggcaaatgctttcgcagtagttc | 5-methyl cytosine at residues 2 & 3 |
| 58 | cggtgaaactgcgaatggctcattaaaac | 5-methyl cytosine at residues 9, 12, & 19 |
| 59 | atgccctgtattgttatttctcgtc | 5-methyl cytosine at residues 4-6, 20, & 22 |
| | | Propyne dU at residues 2, 7, 12, 15, 18, 21 |
| 62 | tgcctaaaacttccctgtatta | 5-methyl cytosine at residues 3, 4, 10, & 13-15 |
| | | Propyne dU at residues 1, 5, 11, 18, & 21 |
| 63 | ctgctatgactgaggctgaata | 5-methyl cytosine at residues 4, 10, & 16 |
| | | Propyne dU at residues 2, 5, 7, 17, & 21 |
| 64 | gcatcacagacctgttattgccta | 5-methyl cytosine at residues 2, 5, 7, 11, & 12 |
| 65 | tctgtcttcttagagggactttgtatg | 5-methyl cytosine at residues 2, 6, 9, 19 |
| | | Propyne dU at residues 3, 5, 10, 20, 22, 24, & 26 |
| 66 | tctgtcttcttagagggactttgtatg | 5-methyl cytosine at residues 2, 6, 9, 19 |
| | | Propyne dU at residues 3, 5, 10, 20, 22, 24, & 26 |
| 67 | cctatcagctttagacggtagggtattgg | 5-methyl cytosine at residues 2, 6, 9, & 16 |
| | | Propyne dU at residues 5, 19, & 26 |
| 68 | ttatctgtcttcttagagggactttgta | 5-methyl cytosine at residues 5, 9, 12, & 22 |
| | | Propyne dU at residues 2, 4, 8, 10, 14, 23, & 25 |
| 69 | ctcataataactttacggatcaca | 5-methyl cytosine at residues 3 & 11 |
| 70 | cgtatttaactgtcagaggtg | 5-methyl cytosine at residues 10 & 14 |
| 71 | cgcaaattacccaatgaaaa | 5-methyl cytosine at residues 10 & 11 |
| 72 | ccctactgtcgttcttgat | 5-methyl cytosine at residue 3 |
| 73 | cgtcagcaggttccacgacaaga | 5-methyl cytosine at residues 4, 7, 13, 14, 16, & |
| | | 19 |
| 74 | ggtgmgcgggcatgcatggctt | 5-methyl cytosine at residues 7, 11, 15, & 20 |
| 75 | ggtgcgcgggcatgcatggctt | 5-methyl cytosine at residues 7, 11, 15, & 20 |
| 76 | ggtgagcgggcatgcatggctt | 5-methyl cytosine at residues 7, 11, 15, & 20 |
| 77 | cgtcagcaggttccacgacaaga | 5-methyl cytosine at residues 4, 7, 13, 14, 16, & |
| | | 19 |
| 78 | gtgagttaggatgccacgacaattg | 5-methyl cytosine at residues 14, 15, 17, & 20 |
| | | Propyne dU at residues7, 12, 23, & 24 |
| 79 | attctaagtgagttaggatgccacg | 5-methyl cytosine at residues 4, 21, 22, & 24 |
| | | Propyne dU at residues 2, 3, 5, 9, 13, 14, & 19 |
| 80 | acaaaatggccaattcattcaatgaattga | 5-methyl cytosine at residues 2, 10, 11, 16, & 20 |
| | | Propyne dU at residues 7, 14, 15, 18, 19, 23, 27, & |
| | | 28 |
| 81 | attgttactctggaaggattt | 5-methyl cytosine at residues 8 & 10 |
| 82 | attgttactctggaaggattt | 5-methyl cytosine at residues 8 & 10 |
| | | Propyne dU at residues 6, 11, & 20 |
| 83 | atctaatcgtcttcaaaccc | 5-methyl cytosine at residues 3, 8, 11, & 14 |
| 84 | agcctgctaaatagacataaga | 5-methyl cytosine at residues 3, 4, 7, & 16 |
| | | Propyne dU at residues 8, 12, & 18 |
| 85 | aagctcagcaacatgaagg | 5-methyl cytosine at residues 4 & 6 |
| 86 | cagaaattctcattggttacttg | 5-methyl cytosine at residues 1, 9, 11, & 20 |
| | | Propyne dU at residues 7, 13, 17, & 22 |
| 87 | aaatccttccagagtaacaat | 5-methyl cytosine at residues 5, 6, & 9 |
| 88 | actgcaacaactctaatatacac | 5-methyl cytosine at residue 2 |
| 89 | acctgctaaatagacataaga | 5-methyl cytosine at residues 2, 3, 6, & 15 |
| | | Propyne dU at residues 7, 11, & 17 |
| 90 | ctgtcagaggtgaaattctt | 5-methyl cytosine at residues 1, 5, & 18 |
| 91 | cggaatcgaaccctaattccc | 5-methyl cytosine at residues 1, 7, 11, & 13 |
| 92 | cgacgctctccccaagga | 5-methyl cytosine at residnes 1, 4, 6, & 8 |
| 93 | cgcaaattacccaatgaaaa | 5-methyl cytosine at residues 1, 3, 10, & 12 |
| | | Propyne dU at residues 7, 8, & 15 |
| SEQ ID | | |
| NO: | Sequence (5′→3′) | Exemplary Detectable Labels |
| 7 | acaaaatggccaattcattcaatgaattga | 5′ CalOrange560 |
| | | 3′ BHQ-1 |
| 10 | agacgaactactgcgaaagcatttgccaa | 5′ Quasar 670 |
| | | 3′ BHQ-2 |
| 12 | aggaccctccgatcggttgca | 5′ Quasar 670 |
| | | 3′ BHQ-2 |
| 16 | atgccctgtattgttatttctcgtc | 5′ Quasar 670 |
| | | 3′ BHQ-2 |
| 17 | attctaagtgagttaggatgccacg | 5′ CalOrange560 |
| | | 3′ BHQ-1 |
| 24 | cctatcagctttagacggtagggtattgg | 5′ Fluorescein |
| | | BHQ-1 at residue 12 |
| | | 3′ Reverse Polarity C |
| 30 | cggtgaaactgcgaatggctcattaaaac | 5′ Quasar 670 |
| | | 3′ BHQ-2 |
| 32 | cgtcagcaggttccacgacaaga | 5′ CalRed610 |
| | | 3′ BHQ-2 |
| 34 | ctgctatgactgaggctgaata | 5′ HEX |
| | | BHQ-1 at residue 11 |
| | | 3′ Reverse Polarity dC |
| 39 | ggtgagcgggcatgcatggctt | 5′ CalRed610 |
| | | 3′ BHQ-2 |
| 40 | ggtgcgcgggcatgcatggctt | 5′ CalRed610 |
| | | 3′ BHQ-2 |
| 41 | ggtgmgcgggcatgcatggctt | 5′ CalRed610 |
| | | 3′ BHQ-2 |
| 44 | gtgagttaggatgccacgacaattg | 5′ CalOrange560 |
| | | 3′ BHQ-1 |
| 45 | taaatgccctgtattgttatttctcgtca | 5′ Quasar 670 |
| | | 3′ BHQ-2 |
| 47 | tccttggcaaatgctttcgcagtagttc | 5′ Quasar 670 |
| | | 3′ BHQ-2 |
| 48 | tctgtcttcttagagggactttgtatg | S′ Fluorescein |
| | | 3′ BHQ-1 |
| 48 | tctgtcttcttagagggactttgtatg | 5′ Fluorescein |
| | | BHQ-1 at residue 11 |
| | | 3′ Reverse Polarity dC |
| 50 | tggtgccagcagccgc | 5′ Quasar 670 |
| | | 3′ BHQ-2 |
| 52 | ttatctgtcttcttagagggactttgta | 5′ Fluorescein |
| | | BHQ-1 at residue 11 |
| | | 3′ Reverse Polarity dC |
| 55 | tggtgccagcagccgc | 5′ Quasar 670 |
| | | 3′ BHQ-2 |
| 56 | taaatgccctgtattgttatttctcgtca | 5′ Quasar 670 |
| | | 3′ BHQ-2 |
| 57 | tccttggcaaatgctttcgcagtagttc | 5′ Quasar 670 |
| | | 3′ BHQ-2 |
| 58 | cggtgaaactgcgaatggctcattaaaac | 5′ Quasar 670 |
| | | 3′ BHQ-2 |
| 59 | atgccctgtattgttatttctcgtc | 5′ Quasar 670 |
| | | 3′ BHQ-2 |
| 63 | ctgctatgactgaggctgaata | 5′ HEX |
| | | BHQ-1 at residue 11 |
| | | 3′ Reverse Polarity dC |
| 65 | tctgtcttcttagagggactttgtatg | S′ Fluorescein |
| | | 3′ BHQ-1 |
| 66 | tctgtcttcttagagggactttgtatg | 5′ Fluorescein |
| | | BHQ-1 at residue 11 |
| | | 3′ Reverse Polarity dC |
| 67 | cctatcagctttagacggtagggtattgg | 5′ Fluorescein |
| | | BHQ-1 at residue 12 |
| | | 3′ Reverse Polarity dC |
| 68 | ttatctgtcttcttagagggactttgta | 5′ Fluorescein |
| | | BHQ-1 at residue 11 |
| | | 3′ Reverse Polarity dC |
| 73 | cgtcagcaggttccacgacaaga | 5′ CalRed610 |
| | | 3′ BHQ-2 |
| 74 | ggtgmgcgggcatgcatggctt | 5′ CalRed610 |
| | | 3′ BHQ-2 |
| 75 | ggtgcgcgggcatgcatggctt | 5′ CalRed610 |
| | | 3′ BHQ-2 |
| 76 | ggtgagcgggcatgcatggctt | 5′ CalRed610 |
| | | 3′ BHQ-2 |
| 77 | cgtcagcaggttccacgacaaga | 5′ CalRed610 |
| | | 3′ BHQ-2 |
| 78 | gtgagttaggatgccacgacaattg | 5′ CalOrange560 |
| | | 3′ BHQ-1 |
| 79 | attctaagtgagttaggatgccacg | 5′ CalOrange560 |
| | | 3′ BHQ-1 |
| 80 | acaaaatggccaattcattcaatgaattga | 5′ CalOrange560 |
| | | 3′ BHQ-1 |

From the foregoing, it will be appreciated that, although specific embodiments have been described herein for purposes of illustration, various modifications may be made without deviating from the spirit and scope of the disclosure. All publications, patents, and patent applications cited herein are hereby incorporated by reference in their entireties for all purposes.

1. A composition or kit for determining the presence or absence of at least one enteric parasite in a sample, wherein the at least one enteric parasite is selected from the group consisting of Cryptosporidium spp., Entamoeba histolytica, Giardia lamblia, and Cyclospora cayetanensis, said composition or kit comprising a set of oligonucleotides comprising at least one of (a)-(d):
(a) a Cryptosporidium-specific amplification oligomer set capable of amplifying a target region of a Cryptosporidium spp. target nucleic acid, wherein the Cryptosporidium-specific amplification oligomer set comprises first and second Cryptosporidium-specific amplification oligomers respectively comprising target-hybridizing sequences as shown in (i) SEQ ID NO:8 and SEQ ID NO:37, including from 0 to 16 nucleotide analogs; (ii) SEQ ID NO:33 and SEQ ID NO:28, including from 0 to 16 nucleotide analogs; (iii) SEQ ID NO:4 and SEQ ID NO:37, including from 0 to 16 nucleotide analogs; (iv) SEQ ID NO:11 and SEQ ID NO:49, including from 0 to 16 nucleotide analogs; (v) SEQ ID NO:4 and SEQ ID NO:49, including from 0 to 16 nucleotide analogs; (vi) SEQ ID NO:11 and SEQ ID NO:37, including from 0 to 16 nucleotide analogs; or (vii) SEQ ID NO:8 and SEQ ID NO:49, including from 0 to 16 nucleotide analogs;
(b) an Entamoeba-specific amplification oligomer set capable of amplifying a target region of an Entamoeba histolytica target nucleic acid, wherein the Entamoeba-specific amplification oligomer set comprises first and second Entamoeba-specific amplification oligomers respectively comprising target-hybridizing sequences as shown in (i) SEQ ID NO:46 and SEQ ID NO:19, including from 0 to 16 nucleotide analogs; (ii) SEQ ID NO:21 and SEQ ID NO:20, including from 0 to 16 nucleotide analogs; (iii) SEQ ID NO:36 and SEQ ID NO:43, including from 0 to 16 nucleotide analogs; or (iv) SEQ ID NO:42 and SEQ ID NO:19, including from 0 to 16 nucleotide analogs;
(c) a Giardia-specific amplification oligomer set capable of amplifying a target region of a Giardia lamblia target nucleic acid, wherein the Giardia-specific amplification oligomer set comprises first and second Giardia-specific amplification oligomers respectively comprising target-hybridizing sequences as shown in (i) SEQ ID NO:25 and SEQ ID NO:2, including from 0 to 16 nucleotide analogs; (ii) SEQ ID NO:5 and SEQ ID NO:29, including from 0 to 16 nucleotide analogs; (iii) SEQ ID NO:6 and SEQ ID NO:29, including from 0 to 16 nucleotide analogs; or (iv) SEQ ID NO:25 and SEQ ID NO:3, including from 0 to 16 nucleotide analogs; and
(d) a Cyclospora-specific amplification oligomer set capable of amplifying a target region of a Cyclospora cayetanensis target nucleic acid, wherein the Cyclospora-specific amplification oligomer set comprises first and second Cyclospora-specific amplification oligomers respectively comprising target-hybridizing sequences as shown in (i) SEQ ID NO:26 and SEQ ID NO:14, including from 0 to 16 nucleotide analogs; (ii) SEQ ID NO:23 and SEQ ID NO:18, including from 0 to 16 nucleotide analogs; (iii) SEQ ID NO:31 and SEQ ID NO:22, including from 0 to 16 nucleotide analogs; (iv) SEQ ID NO:35 and SEQ ID NO:13, including from 0 to 16 nucleotide analogs; (v) SEQ ID NO:38 and SEQ ID NO:51, including from 0 to 16 nucleotide analogs; or (vi) SEQ ID NO:1 and SEQ ID NO:9, including from 0 to 16 nucleotide analogs.

2. The composition or kit of claim 1, wherein the set of oligonucleotides comprises the Cryptosporidium-specific amplification oligomer set.

3. The composition or kit of claim 2, wherein the set of oligonucleotides further comprises a Cryptosporidium-specific detection probe comprising a target-hybridizing sequence substantially corresponding to the nucleotide sequence of
SEQ ID NO:52 or SEQ ID NO:48 if the Cryptosporidium-specific amplification oligomer set comprises first and second Cryptosporidium-specific amplification oligomers respectively comprising target-hybridizing sequences as shown in (i) SEQ ID NO:8 and SEQ ID NO:37, including from 0 to 16 nucleotide analogs, (ii) SEQ ID NO:4 and SEQ ID NO:37, including from 0 to 16 nucleotide analogs, (iii) SEQ ID NO:11 and SEQ ID NO:49, including from 0 to 16 nucleotide analogs, (iv) SEQ ID NO:4 and SEQ ID NO:49, including from 0 to 16 nucleotide analogs, (v) SEQ ID NO:11 and SEQ ID NO:37, including from 0 to 16 nucleotide analogs, or (vi) SEQ ID NO:8 and SEQ ID NO:49, including from 0 to 16 nucleotide analogs; or
SEQ ID NO:24 if the Cryptosporidium-specific amplification oligomer set comprises first and second Cryptosporidium-specific amplification oligomers respectively comprising target-hybridizing sequences as shown in SEQ ID NO:33 and SEQ ID NO:28, including from 0 to 16 nucleotide analogs.

4. The composition or kit of claim 1, wherein the set of oligonucleotides comprises the Entamoeba-specific amplification oligomer set.

5. The composition or kit of claim 4, wherein the set of oligonucleotides further comprises an Entamoeba-specific detection probe comprising a target-hybridizing sequence substantially corresponding to the nucleotide sequence of
SEQ ID NO:17 if the Entamoeba-specific amplification oligomer set comprises first and second Entamoeba-specific amplification oligomers respectively comprising target-hybridizing sequences as shown in SEQ ID NO:46 and SEQ ID NO:19, including from 0 to 16 nucleotide analogs;
SEQ ID NO:34 if the Entamoeba-specific amplification oligomer set comprises first and second Entamoeba-specific amplification oligomers respectively comprising target-hybridizing sequences as shown in SEQ ID NO:21 and SEQ ID NO:20, including from 0 to 16 nucleotide analogs;
SEQ ID NO:7 if the Entamoeba-specific amplification oligomer set comprises first and second Entamoeba-specific amplification oligomers respectively comprising target-hybridizing sequences as shown in SEQ ID NO:36 and SEQ ID NO:43 including from 0 to 16 nucleotide analogs; or
SEQ ID NO:44 if the Entamoeba-specific amplification oligomer set comprises first and second Entamoeba-specific amplification oligomers respectively comprising target-hybridizing sequences as shown in SEQ ID NO:42 and SEQ ID NO:19, including from 0 to 16 nucleotide analogs.

6. The composition or kit of claim 1, wherein the set of oligonucleotides comprises the Giardia-specific amplification oligomer set.

7. The composition or kit of claim 6, wherein the set of oligonucleotides further comprises a Giardia-specific detection probe comprising a target-hybridizing sequence substantially corresponding to the nucleotide sequence of
SEQ ID NO:39, SEQ ID NO:40, or SEQ ID NO:41 if the Giardia-specific amplification oligomer set comprises first and second Giardia-specific amplification oligomers respectively comprising target-hybridizing sequences as shown in (i) SEQ ID NO:25 and SEQ ID NO:2 or (ii) SEQ ID NO:25 and SEQ ID NO:3, including from 0 to 16 nucleotide analogs; or
SEQ ID NO:32 if the Giardia-specific amplification oligomer set comprises first and second Giardia-specific amplification oligomers respectively comprising target-hybridizing sequences as shown in (i) SEQ ID NO:5 and SEQ ID NO:29 or (ii) SEQ ID NO:6 and SEQ ID NO:29, including from 0 to 16 nucleotide analogs.

8. The composition or kit of claim 1, wherein the set of oligonucleotides comprises the Cyclospora-specific amplification oligomer set.

9. The composition or kit of claim 8, wherein the set of oligonucleotides further comprises a Cyclospora-specific detection probe comprising a target-hybridizing sequence substantially corresponding to the nucleotide sequence of
SEQ ID NO:16 or SEQ ID NO:45 if the Cyclospora-specific amplification oligomer set comprises first and second Cyclospora-specific amplification oligomers respectively comprising target-hybridizing sequences as shown in SEQ ID NO:26 and SEQ ID NO:14, including from 0 to 16 nucleotide analogs;
SEQ ID NO:12 if the Cyclospora-specific amplification oligomer set comprises first and second Cyclospora-specific amplification oligomers respectively comprising target-hybridizing sequences as shown in SEQ ID NO:23 and SEQ ID NO:18, including from 0 to 16 nucleotide analogs;
SEQ ID NO:10 if the Cyclospora-specific amplification oligomer set comprises first and second Cyclospora-specific amplification oligomers respectively comprising target-hybridizing sequences as shown in SEQ ID NO:31 and SEQ ID NO:22;
SEQ ID NO:47 if the Cyclospora-specific amplification oligomer set comprises first and second Cyclospora-specific amplification oligomers respectively comprising target-hybridizing sequences as shown in SEQ ID NO:35 and SEQ ID NO:13, including from 0 to 16 nucleotide analogs;
SEQ ID NO:30 if the Cyclospora-specific amplification oligomer set comprises first and second Cyclospora-specific amplification oligomers respectively comprising target-hybridizing sequences as shown in SEQ ID NO:38 and SEQ ID NO:51, including from 0 to 16 nucleotide analogs; or
SEQ ID NO:50 if the Cyclospora-specific amplification oligomer set comprises first and second Cyclospora-specific amplification oligomers respectively comprising target-hybridizing sequences as shown in SEQ ID NO:1 and SEQ ID NO:9, including from 0 to 16 nucleotide analogs.

10. The composition or kit of claim 1, wherein the set of oligonucleotides comprises at least two of the Cryptosporidium-specific amplification oligomer set, the Entamoeba-specific amplification oligomer set, the Giardia-specific amplification oligomer set, and the Cyclospora-specific amplification oligomer set.

11. The composition or kit of claim 1, wherein the set of oligonucleotides comprises at least three of the Cryptosporidium-specific amplification oligomer set, the Entamoeba-specific amplification oligomer set, the Giardia-specific amplification oligomer set, and the Cyclospora-specific amplification oligomer set.

12. The composition or kit of claim 1, wherein the set of oligonucleotides comprises the Cryptosporidium-specific amplification oligomer set, the Entamoeba-specific amplification oligomer set, the Giardia-specific amplification oligomer set, and the Cyclospora-specific amplification oligomer set.

13. The composition or kit of claim 3, wherein the detection probe comprises a detectable label.

14. The composition or kit of claim 13, wherein the detectable label is a fluorescent or chemiluminescent label.

15. The composition or kit of claim 13, wherein the detectable label is a fluorescent label and the detection probe further comprises a non-fluorescent quencher.

16-18. (canceled)

19. A reaction mixture for determining the presence or absence of at least one enteric parasite in a sample, said reaction mixture comprising a set of oligonucleotides as specified in claim 1.

20. (canceled)

21. A method for determining the presence or absence of at least one enteric parasite in a sample, wherein the at least one enteric parasite is selected from the group consisting of Cryptosporidium spp., Entamoeba histolytica, Giardia lamblia, and Cyclospora cayetanensis, the method comprising:
(1) contacting a sample, said sample suspected of containing the at least one enteric parasite, with an oligomer combination capable of amplifying a target region of Cryptosporidium spp., Entamoeba histolytica, Giardia lamblia, and Cyclospora cayetanensis target nucleic acid, said oligomer combination comprising
(a) a Cryptosporidium-specific amplification oligomer set capable of amplifying a target region of a Cryptosporidium spp. target nucleic acid, wherein the Cryptosporidium-specific amplification oligomer set comprises first and second Cryptosporidium-specific amplification oligomers respectively comprising target-hybridizing sequences as shown in (i) SEQ ID NO:8 and SEQ ID NO:37, including from 0 to 16 nucleotide analogs; (ii) SEQ ID NO:33 and SEQ ID NO:28, including from 0 to 16 nucleotide analogs; (iii) SEQ ID NO:4 and SEQ ID NO:37, including from 0 to 16 nucleotide analogs; (iv) SEQ ID NO:11 and SEQ ID NO:49, including from 0 to 16 nucleotide analogs; (v) SEQ ID NO:4 and SEQ ID NO:49, including from 0 to 16 nucleotide analogs; (vi) SEQ ID NO:11 and SEQ ID NO:37, including from 0 to 16 nucleotide analogs; or (vii) SEQ ID NO:8 and SEQ ID NO:49, including from 0 to 16 nucleotide analogs; and/or
(b) an Entamoeba-specific amplification oligomer set capable of amplifying a target region of an Entamoeba histolytica target nucleic acid, wherein the Entamoeba-specific amplification oligomer set comprises first and second Entamoeba-specific amplification oligomers respectively comprising target-hybridizing sequences as shown in (i) SEQ ID NO:46 and SEQ ID NO:19, including from 0 to 16 nucleotide analogs; (ii) SEQ ID NO:21 and SEQ ID NO:20, including from 0 to 16 nucleotide analogs; (iii) SEQ ID NO:36 and SEQ ID NO:43, including from 0 to 16 nucleotide analogs; or (iv) SEQ ID NO:42 and SEQ ID NO:19, including from 0 to 16 nucleotide analogs; and/or
(c) a Giardia-specific amplification oligomer set capable of amplifying a target region of a Giardia lamblia target nucleic acid, wherein the Giardia-specific amplification oligomer set comprises first and second Giardia-specific amplification oligomers respectively comprising target-hybridizing sequences as shown in (i) SEQ ID NO:25 and SEQ ID NO:2, including from 0 to 16 nucleotide analogs; (ii) SEQ ID NO:5 and SEQ ID NO:29% including from 0 to 16 nucleotide analogs; (iii) SEQ ID NO:6 and SEQ ID NO:29% including from 0 to 16 nucleotide analogs; or (iv) SEQ ID NO:25 and SEQ ID NO:3, including from 0 to 16 nucleotide analogs; and/or
(d) a Cyclospora-specific amplification oligomer set capable of amplifying a target region of a Cyclospora cayetanensis target nucleic acid, wherein the Cyclospora-specific amplification oligomer set comprises first and second Cyclospora-specific amplification oligomers respectively comprising target-hybridizing sequences as shown in (i) SEQ ID NO:26 and SEQ ID NO:14, including from 0 to 16 nucleotide analogs; (ii) SEQ ID NO:23 and SEQ ID NO:18, including from 0 to 16 nucleotide analogs; (iii) SEQ ID NO:31 and SEQ ID NO:22, including from 0 to 16 nucleotide analogs; (iv) SEQ ID NO:35 and SEQ ID NO:13, including from 0 to 16 nucleotide analogs; (v) SEQ ID NO:38 and SEQ ID NO:51, including from 0 to 16 nucleotide analogs; (vi) SEQ ID NO:1 and SEQ ID NO:9, including from 0 to 16 nucleotide analogs;
(2) performing an in vitro nucleic acid amplification reaction, wherein any Cryptosporidium spp., Entamoeba histolytica, Giardia lamblia, and/or Cyclospora cayetanensis target nucleic acid present in the sample is used as a template for generating one or more amplification products corresponding to the Cryptosporidium spp., Entamoeba histolytica, Giardia lamblia, and/or Cyclospora cayetanensis target regions; and
(3) detecting the presence or absence of the one or more amplification products, thereby determining the presence or absence of the at least one enteric parasite in the sample.

22. The method of claim 21, wherein
if the sample is contacted with the Cryptosporidium-specific amplification oligomer set and any Cryptosporidium spp. target nucleic acid present in the sample is used as a template for generating a Cryptosporidium spp. amplification product corresponding to the Cryptosporidium spp. target region, then the detecting step (3) comprises contacting the in vitro nucleic acid amplification reaction with a Cryptosporidium-specific detection probe configured to specifically hybridize to the Cryptosporidium spp. amplification product; and/or;
if the sample is contacted with the Entamoeba-specific amplification oligomer set comprising the first and second Entamoeba-specific amplification oligomers and any Entamoeba histolytica target nucleic acid present in the sample is used as a template for generating a Entamoeba histolytica amplification product corresponding to the Entamoeba histolytica target region, then the detecting step (3) comprises contacting the in vitro nucleic acid amplification reaction with a Entamoeba-specific detection probe configured to specifically hybridize to the Entamoeba histolytica amplification product; and/or
if the sample is contacted with the Giardia-specific amplification oligomer set and any Giardia lamblia target nucleic acid present in the sample is used as a template for generating a Giardia lamblia amplification product corresponding to the Giardia lamblia target region; then the detecting step (3) comprises contacting the in vitro nucleic acid amplification reaction with a Giardia-specific detection probe configured to specifically hybridize to the Giardia lamblia amplification product; and/or
if the sample is contacted with the Cyclospora-specific amplification oligomer set and any Cyclospora cayetanensis target nucleic acid present in the sample is used as a template for generating a Cyclospora cayetanensis amplification product corresponding to the Cyclospora cayetanensis target region, then the detecting step (3) comprises contacting the in vitro nucleic acid amplification reaction with a Cyclospora-specific detection probe configured to specifically hybridize to the Cyclospora cayetanensis amplification product.

23. (canceled)

24. (canceled)

25. The method of claim 21, wherein the method is a multiplex method for detecting the presence or absence of each of Cryptosporidium spp., Entamoeba histolytica, Giardia lamblia, and Cyclospora cayetanensis.

26. (canceled)

27. A method for synthesizing a pair of oligonucleotides, comprising synthesizing a first oligonucleotide and synthesizing a second oligonucleotide,
wherein each of the synthesizing the first oligonucleotide and the synthesizing the second oligonucleotide comprises the steps of:
(a) obtaining a solid support comprising at least one nucleobase residue, wherein the at least one nucleobase residue is covalently bound at a 3′ position to the solid support;
(b) coupling a 5′ position of the nucleobase residue furthest from the solid support to a 3′ position of another nucleobase residue;
(c) repeating step (b) at least 15 additional times, thereby generating at least 17 contiguous nucleobase residues coupled to the solid support; and
(d) cleaving the at least 17 contiguous nucleobase residues generated in step (c), thereby obtaining the oligonucleotide, and
wherein the first oligonucleotide and the second oligonucleotide respectively comprise the nucleotide sequences of any one of
SEQ ID NO:8 and SEQ ID NO:37, including from 0 to 16 nucleotide analogs;
SEQ ID NO:33 and SEQ ID NO:28, including from 0 to 16 nucleotide analogs;
SEQ ID NO:4 and SEQ ID NO:37, including from 0 to 16 nucleotide analogs;
SEQ ID NO:11 and SEQ ID NO:49, including from 0 to 16 nucleotide analogs;
SEQ ID NO:4 and SEQ ID NO:49, including from 0 to 16 nucleotide analogs;
SEQ ID NO:11 and SEQ ID NO:37, including from 0 to 16 nucleotide analogs;
SEQ ID NO:8 and SEQ ID NO:49, including from 0 to 16 nucleotide analogs;
SEQ ID NO:46 and SEQ ID NO:19, including from 0 to 16 nucleotide analogs;
SEQ ID NO:21 and SEQ ID NO:20, including from 0 to 16 nucleotide analogs;
SEQ ID NO:36 and SEQ ID NO:43, including from 0 to 16 nucleotide analogs;
SEQ ID NO:42 and SEQ ID NO:19, including from 0 to 16 nucleotide analogs;
SEQ ID NO:25 and SEQ ID NO:2, including from 0 to 16 nucleotide analogs;
SEQ ID NO:5 and SEQ ID NO:29, including from 0 to 16 nucleotide analogs;
SEQ ID NO:6 and SEQ ID NO:29, including from 0 to 16 nucleotide analogs;
SEQ ID NO:25 and SEQ ID NO:3, including from 0 to 16 nucleotide analogs;
SEQ ID NO:26 and SEQ ID NO:14, including from 0 to 16 nucleotide analogs;
SEQ ID NO:23 and SEQ ID NO:18, including from 0 to 16 nucleotide analogs;
SEQ ID NO:31 and SEQ ID NO:22, including from 0 to 16 nucleotide analogs;
SEQ ID NO:35 and SEQ ID NO:13, including from 0 to 16 nucleotide analogs;
SEQ ID NO:38 and SEQ ID NO:51, including from 0 to 16 nucleotide analogs; or
SEQ ID NO:1 and SEQ ID NO:9, including from 0 to 16 nucleotide analogs.
